(19) United States
(12) Patent Application Publication
(10) Pub. No.: US 2002/0081665 A1
(43) Pub. Date: Jun. 27, 2002

(54) Bryostatins, bryopyrans and polyketides: compositions and methods

(76) Inventors: Margo Haygood, La Jolla, CA (US); Seana K. Davidson, La Jolla, CA (US); Scott W. Allen, San Diego, CA (US); Mark Hildebrand, La Jolla, CA (US)

Correspondence Address: Schwegman, Lundberg, Woessner & Kluth, P.A., P.O. Box 2938, Minneapolis, MN, 55402, US

(21) Appl. No.: 09/775,938

(22) Filed: Jan. 31, 2001

Related U.S. Application Data
(63) Continuation of application No. PCT/US00/21326, filed on Aug. 4, 2000.
(60) Provisional application No. 60/147,283, filed on Aug. 4, 1999.

(51) Int. Cl. C12Q 1/68; C07H 17/08; C07H 21/04; C12P 19/62; C12N 1/21

(52) U.S. Cl. 435/76; 536/7.1; 536/23.2; 435/252.3; 435/6; 514/27; 514/28

(57) ABSTRACT

The present invention recognizes that marine organisms comprise nucleic acid molecules that encode polypeptides that catalyze the synthesis of bioactive compounds, such as polyketides including bryopyran rings, such as bryostatins. One aspect of the present invention is a composition including at least one nucleic acid molecule that encodes at least one polypeptide that catalyzes at least one step in the synthesis of at least one polyketide such as a bryopyran ring, wherein said at least one nucleic acid molecule is derived from at least one marine organism. A second aspect of the present invention is a composition including a library of nucleic acid molecules of the present invention. These nucleic acid molecules can be used in a combinatorial biosynthesis of polyketides, bryopyran rings and bryostatins.

### CROSS-REFERENCE TO RELATED APPLICATIONS

This application claims the benefit of priority of U.S. Provisional Patent application No. 60/147,283 to Haygood et al., filed Aug. 4, 1999, which is incorporated by reference herein in its entirety.

### STATEMENT OF GOVERNMENT RIGHTS

This invention was made at least in part with government support awarded by the National Institutes of Health (NCI/NIH 2R44-CA58158-02A3 to CalBioMarine Technologies, Inc.) and California Sea Grant (RMP-61). The United States Government may have certain rights in the invention.

### TECHNICAL FIELD

The present invention generally relates to polyketides, including bryopyran rings, such as bryostatins, and methods of making polyketides, bryopyran rings and bryostatins.

### BACKGROUND OF THE INVENTION

Polyketide synthases (PKS) are enzymes that catalyze the synthesis of polyketides, a class of compounds that have diverse activities, including a variety of bioactivities such as anticancer and immunomodulatory activity. Polyketides are created by the sequential condensation of acetate or other simple fatty acid units in a manner analogous to fatty acid synthesis. There are two types of cyclic polyketides, complex and aromatic, where bryostatin are classified as complex polyketides. PKS enzymes are classified as Type I (PKS-I), having multiple active sites on a single polypeptide, or Type II (PKS-II), having singe active site polypeptides that form a complex.

Bryostatins are a set of bioactive complex polyketides based on a bryopyran ring structure whose synthetic pathways have evaded elucidation. Bryostatins are found in invertebrates of the genus Bugula, such as Bugula neritina, and are believed to give the marine invertebrates a competitive advantage in the environment due to their toxicity. This toxicity also makes the bryostatins attractive pharmaceutical agents. Bryostatins, such as bryostatin 1, have been used extensively in clinical trials for the treatment of a variety of cancers, carcinomas and lymphomas. However, the only source for bryostatins is from natural collections or aquaculture. These sources can be unpredictable due to environmental conditions, such as El Nino events, and overharvesting of wild populations. Thus, there exists a need for a reliable and economical source for bryostatins.

The present invention addresses this and other needs by identifying and characterizing genes that are involved in the synthesis of polyketides such as bryopyran ring structures, such as bryostatins, and expressing these genes in heterologous organisms. These genes can be used to produce base structures, such as bryopyran rings, that can form the basis of combinatorial chemistry to produce a wide variety of compounds, including those made using combinatorial biosynthetic procedures. These compounds can be screened for a variety of bioactivities including anticancer activity. The present invention provides related benefits as well.

### SUMMARY OF THE INVENTION

The present invention recognizes that marine organisms contain nucleic acid molecules that encode polypeptides that catalyze the synthesis of bioactive compounds, such as polyketides including bryopyran rings, such as bryostatins.

One aspect of the present invention is a composition including at least one isolated nucleic acid molecule that encodes at least one polypeptide that catalyzes at least one step in the synthesis of at least one polyketide such as a bryopyran ring, wherein said at least one nucleic acid molecule is derived from at least one marine organism.

A second aspect of the present invention is a composition including a library of nucleic acid molecules of the present invention. These nucleic acid molecules can be used in a combinatorial biosynthesis of polyketides, bryopyran rings and bryostatins.

A third aspect of the present invention is a composition including at least one isolated polypeptide that catalyzes at least one step in the synthesis of at least one polyketide such as a bryopyran ring, wherein said at least one polypeptide is derived from at least one marine organism.

A fourth aspect of the present invention is a composition including a library of polypeptides of the present invention.

A fifth aspect of the present invention is a method of making a composition, including providing at least one nucleic acid of the present invention and synthesizing at least one polyketide such as a bryopyran ring.

A sixth aspect of the present invention is a composition made using a nucleic acid of the present invention.

A seventh aspect of the present invention is a method of making a composition including providing at least one polypeptide of the present invention and synthesizing at least one polyketide such as a bryopyran ring.

An eighth aspect of the present invention is a composition made using a polypeptide of the present invention.

A ninth aspect of the present invention is a method for identifying at least one nucleic acid molecule encoding at least one activity of a PKS including contacting a nucleic acid molecule of the present invention with a sample, and identifying nucleic acid molecules in said sample that hybridize with a nucleic acid of the present invention.

A tenth aspect of the present invention is a nucleic acid molecule identified by a method of the present invention.

An eleventh aspect of the present invention is a composition comprising a library of nucleic acid molecules of the present invention.

A twelfth aspect of the present invention is a method for identifying a bioactive compound including contacting a compound made or identified using a nucleic acid molecule of the present invention and determining the bioactivity of the compound.

A thirteenth aspect of the present invention is a method for identifying a bioactive compound including contacting a compound made or identified using a polypeptide of the present invention and determining the bioactivity of said compound.

A fourteenth aspect of the present invention is a preparation of bacteria from a Bugula that include PKS genes.

A fifteenth aspect of the present invention is a polyketide, bryopyran ring or bryostatin present in Bugula pacifica.

### BRIEF DESCRIPTION OF THE FIGURES

FIG. 1 depicts structures of illustrative bryostatins.

FIG. 2 depicts reactions catalyzed by type I Polyketide synthase EryA.

FIG. 3 depicts an expected domain structure of a bryopyran synthase and predicted structures.

FIG. 4 depicts a method for expression of bryostatin synthase genes in S. venezuelae.

FIG. 5 depicts a method for intermodular PCR for PKS genes.

FIG. 6 depicts PCR amplification products of various Bugula, including B. neritina from California and North Carolina using KSa PCR primers of the present invention.

FIG. 7A to FIG. 7D depict the effects of treatment of B. neritina with gentamicin and the production of bryostatins.

FIG. 8 depicts competitive PCR analysis of DNA preparations from B. neritina tips. Two different DNA preparation were subjected to competitive PCR using a clone of KSa with an internal deletion. Samples were electrophoresed on a 1.5% agarose gel. Lanes 1 and 14 are the “1 Kb ladder” molecular weight marker from Bethesda Research Labs. Lanes 2, 3, and 4 are 10⁻⁴, 10⁻⁵, and 10⁻⁶ dilutions, respectively, of competitor DNA alone. Lane 5 is an unfractionated DNA prep with no competitor, lanes 6-8 are 10⁻⁴, 10⁻⁵, and 10⁻⁶ dilutions, respectively, of the competitor DNA mixed with the unfractionated DNA prep. Lane 9 is amplification of the fractionated DNA prep without competitor, and lanes 10-12 are fractionated 10⁻⁴, 10⁻⁵, and 10⁻⁶ dilutions, respectively, of the competitor DNA mixed with the fractionated DNA prep. Lane 13 is a control of B. neritina larval DNA with no competitor. Equivalent amounts of amplification comparing the competitor and full-length product are visible in the 10⁻⁵ dilution in lane 7, and the 10⁻⁴ dilution in lane 10. This indicates that the representation of E. sertula DNA in the fractionated DNA prep was 10-fold higher the unfractionated sample.

FIG. 9 depicts PCR screening of cosmid clones with KSa-specific primers. PCR was performed on DNA isolated from the clones listed above each lane, using KSa-specific primers, and the products run on a 1.0% agarose gel. Mkr is a 1 Kb ladder, and Cnt. is a control with no added DNA.

FIG. 10 depicts EcoRI restriction digest patterns of cosmids 2A, 3A, 4A, and 6A. Digests were run on a 0.6% agarose gel. Mkr is a 1 Kb ladder, clones are indicated above the lanes.

FIG. 11 depicts hybridization of 6A T3-end probe to EcoRI/SalI digests of cosmids 5A, 5B, 3A and 6A.

FIG. 12 depicts relative location of cosmid clones in relation to PKS gene cluster.

FIG. 13 depicts a clone and sequencing map of PKS cluster region. The positions of clones 3A, 6A, 5A, and 5B are indicated by lines and identified to the right of the lines. Regions sequenced on each clone are denoted by horizontal lines above or below the clone line. For 3A, lines above and below the line indicate that the complete sequence has been obtained on both DNA strands. Regions in the other sequences have been determined mostly on a single strand, and although some sequence on both strands may be present, it is not denoted. T3 or T7 at the ends of clones indicate the orientation of the clone in the cloning vector. Vertical bars represent either the end of a clone or the position of PstI restriction sites. Letters in between vertical bars above the lines in 5A and 5B indicate the name of the cloned restriction fragment sequenced. For 6A, two contigs are noted. For Pst A2/F4/C2 in 5A, it is known that these fragments are contiguous but their precise location in the clone is not known; this is denoted by the arrows. A general region where a portion of the total DNA prep from 5A and 5B has deleted is indicated. The preps contain both full length copies of the insert and deleted copies so the overall map is accurate. The long solid bar below represents the PKS cluster region, PKS homology identified in the sequencing is located by PKS>above the bar. A scale in kbp is at bottom left.

FIG. 14A depicts a contig map of cosmid 3A containing the beginning of the PKS cluster. Clones used for sequencing are listed at left, arrows denote the beginning and end of sequence data obtained for each clone. Bar below indicates the number of base pairs in the contig, which was generated by Sequencher, vers. 3.1.

FIG. 14B is the nucleotide and amino acid sequence of the PKS cluster from clone 3A.

FIG. 15A depicts a contig map of cosmid 6A downstream of 3A.

FIG. 15B is the contig sequences from clone 6A.

FIG. 16A depicts a contig map of cosmid 5A Pst A2/F4/C2 region.

FIG. 16B is a nucleotide sequence from clone 5A.

FIG. 17A depicts contigs of sequence overlapping PstI fragments in 5B.

FIG. 17B is a nucleotide sequence from a portion of clone 5B.

FIG. 18A depicts a contig map of T7 end of cosmid 5A, through 5B Pst A7, to the T3 end of 5B.

FIG. 18B is a nucleotide sequence from a portion of clone 5B.

FIG. 19 depicts PCR amplification products separated by denaturing gradient gel electrophoresis of B. neritina and B. pacifica adult with a universal 16S rRNA primers.

FIG. 20 depicts HPLC profiles of bryostatin-containing extracts from B. neritina and B. pacifica.

FIG. 21 depicts phorbol dibutyrate displacement assays of ethanol extracts of bryozoans including B. pacifica showing binding to PKC.

FIG. 22 depicts exemplary nucleic acid and amino acid sequences.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the laboratory procedures in cell culture, chemistry, microbiology, molecular biology, cell science and cell culture described below are well known and commonly employed in the art. Conventional methods are used for these procedures, such as those provided in the art and various general references (Sambrook et al., Molecular Cloning: A Laboratory Manual, 2nd edition, Cold Spring Harbor Press, Cold Spring Harbor, N.Y. (1989)). Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

“Membrane permeant derivative” refers to a chemical derivative of a compound that increases membrane permeability of the compound. These derivatives are made better able to cross cell membranes because hydrophilic groups are masked to provide more hydrophobic derivatives. Also, the making groups can be designed to be cleaved from the compound within a cell to make the compound more hydrophilic once within the cell. Because the substrate is more hydrophilic than the membrane permeant derivative, it preferentially localizes within the cell (U.S. Pat. No. 5,741,657 to Tsien et al., issued Apr. 21, 1998).

“Isolated polynucleotide” refers to a polynucleotide of genomic, cDNA, PCR or synthetic origin, or some combination thereof, which by virtue of its origin, the isolated polynucleotide (1) is not associated with the cell in which the isolated polynucleotide is found in nature, or (2) is operably linked to a polynucleotide that it is not linked to in nature. The isolated polynucleotide can optionally be linked to promoters, enhancers, or other regulatory sequences.

“Isolated protein” refers to a protein of cDNA, recombinant RNA, or synthetic origin, or some combination thereof, which by virtue of its origin the isolated protein (1) is not associated with proteins normally found within nature, or (2) is isolated from the cell in which it normally occurs, or (3) is isolated free of other proteins from the same cellular source, for example, free of cellular proteins), or (4) is expressed by a cell from a different species, or (5) does not occur in nature.

“Polypeptide” is used herein as a generic term to refer to native protein, fragments, or analogs of a polypeptide sequence.

“Active fragment” refers to a fragment of a parent molecule, such as an organic molecule, nucleic acid molecule, or protein or polypeptide, or combinations thereof, that retains at least one activity of the parent molecule.

“Naturally occurring” refers to the fact that an object can be found in nature. For example, a polypeptide or polynucleotide sequence that is present in an organism, including viruses, that can be isolated from a source in nature and which has not been intentionally modified by man in the laboratory is naturally occurring.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refer to polynucleotide sequences that effect the expression of coding and non-coding sequences to which they are ligated. The nature of such control sequences differs depending upon the host organism; in prokaryotes, such control sequences generally include promoter, ribosomal biding site, and transcription termination sequences; in eukaryotes, generally, such control sequences include promoters and transcription termination sequences. The term control sequences is intended to include components whose presence can influence expression, and can also include additional components whose presence is advantageous, for example, leader sequences and fusion partner sequences.

“Polynucleotide” refers to a polymeric form of nucleotides of a least ten bases in length, either ribonucleotides or deoxyribonucleotides or a modified from of either type of nucleotide. The term includes single and double stranded forms of DNA or RNA.

“Genomic polynucleotide” refers to a portion of the genome.

“Active genomic polynucleotide” or “active portion of a genome” refer to regions of a genome that can be up regulated, down regulated or both, either directly or indirectly, by a biological process.

“Directly” in the context of a biological process or processes, refers to direct causation of a process that does not require intermediate steps, usually caused by one molecule contacting or binding to another molecule (the same type or different type of molecule). For example, molecule A contacts molecule B, which causes molecule B to exert effect X that is part of a biological process.

“Indirectly” in the context of a biological process or precesses, refers to indirect causation that requires intermediate steps, usually caused by two or more direct steps. For example, molecule A contacts molecule B to exert effect X which in turn causes effect Y.

“Sequence identity” refers to the proportion of base matches between two nucleic acid sequences or the proportion of amino acid matches between two amino acid sequences. When sequence identity is expressed as a percentage, for example 50%, the percentage denotes the proportion of matches of the length of sequences from a desired sequence that is compared to some other sequence. Gaps (in either of the two sequences) are permitted to maximize matching; gap lengths of 15 bases or less are usually used, 6 bases or less are preferred with 2 bases or less more preferred. When using oligonuleotides as probes, the sequence identity between the target nucleic acid and the oligonucleotide sequence is preferably not less than 10 target base matches out of 20 (50% identity) and more preferably not less than about 60% identity, 70% identity, 80% identity or 90% identity), and most preferably not less than 95% identity.

“Selectively hybridize” refers to detectably and specifically bind. Polynucleotides, oligonucleotides and fragments thereof selectively hybridize to target nucleic acid strands, under hybridization and wash conditions that minimize appreciable amounts of detectable binding to nonspecific nucleic acids. High stringency conditions can be used to achieve selective hybridization conditions as known in the art. Generally, the nucleic acid sequence identity between the polynucleotides, oligonucleotides, and fragments thereof and a nucleic acid sequence of interest will be at least 30%, and more typically and preferably of at least 40%, 50%, 60%, 70%, 80% or 90%.

Hybridization and washing conditions are typically performed at high stringency according to conventional hybridization procedures. Positive clones are isolated and sequenced. For example, a full length polynucleotide sequence can be labeled and used as a hybridization probe to isolate genomic clones from an appropriate target library as they are known in the art. Typical hybridization conditions and methods for screening plaque lifts and other purposes are known in the art (Benton and Davis, Science 196:180 (1978); Sambrook et al., supra, (1989)).

In particular, moderate and stringent hybridization conditions are well known to the art, see, for example, sections 9.47-9.51 of Sambrook et al. (Molecular Cloning: A Laboratory Manual, Cold Spring Harbor Laboratory, Cold Spring Harbor, N.Y. (1989)). For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% sodium dodecylsulfate (SDS), and 10% dextran sulfate at 42° C., with washes at 42° C. in 0.2×SSC and 0.1% SDS.

Two amino acid sequences have share identity if there is a partial or complete identity between their sequences. For example, 85% identity means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) share identity, as this term is used herein, if they have an alignment score of at least 5 (in standard deviation units) using the program ALIGN with the mutation data matrix and a gap penalty of 6 or greater (Dayhoff, in Atlas of Protein Sequence and Structure, National Biomedical Research Foundation, volume 5, pp. 101-110 (1972) and Supplement 2, pp. 1-10).

“Corresponds to” refers to a polynucleotide sequence that shares identity (for example is identical) to all or a portion of a reference polynucleotide sequence, or that a polypeptide sequence is identical to all or a portion of a reference polypeptide sequence. In contradistinction, the term “complementary to” is used herein to mean that the complementary sequence is homologous to all or a portion of a reference polynucleotide sequence. For illustration, the nucleotide sequence TATAC corresponds to a reference sequence TATAC and is complementary to a reference sequence GTATA.

The following terms are used to describe the sequence relationships between two or more polynucleotides: “reference sequence,” “comparison window,” “sequence identity,” “percentage of sequence identity,” and “substantial identity.” A reference sequence is a defined sequence used as a basis for a sequence comparison; a reference sequence can be a subset of a larger sequence, for example, as a segment of a full length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides can each (1) comprise a sequence (for example a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A comparison window, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window can comprise additions and deletions (for example, gaps) of 20 percent or less as compared to the reference sequence (which would not comprise additions or deletions) for optimal alignment of the two sequences. Optimal alignment of sequences for aligning a comparison window can be conducted by the local identity algorithm (Smith and Waterman, Adv. Appl. Math., 2:482 (1981)), by the identity alignment algorithm (Needleman and Wunsch, J. Mol. Bio., 48:443 (1970)), by the search for similarity method (Pearson and Lipman, Proc. Natl. Acid. Sci. U.S.A. 85:2444 (1988)), by the computerized implementations of these algorithms such as GAP, BESTFIT, FASTA and TFASTA (Wisconsin Genetics Software Page Release 7.0, Genetics Computer Group, Madison, Wis.), or by inspection. Preferably, the best alignment (for example, the result having the highest percentage of identity over the comparison window) generated by the various methods is selected.

“Complete sequence identity” means that two polynucleotide sequences are identical (for example, on a nucleotide-by-nucleotide basis) over the window of comparison.

“Percentage of sequence identity” is calculated by comparing two optimally aligned sequences over the window of comparison, determining the number of positions at which the identical nucleic acid base occurs in both sequences to yield the number of matched positions, dividing the number of matched positions by the total number of positions in the window of comparison (for example, the window size), and multiplying the result by 100 to yield the percentage of sequence identity.

“Substantial identity” as used herein denotes a characteristic of a polynucleotide sequence, wherein the polynucleotide comprises a sequence that has at least 30 percent sequence identity, preferably at least 50 to 60 percent sequence, more usually at least 60 percent sequence identity as compared to a reference sequence over a comparison window of at least 20 nucleotide positions, frequently over a window of at least 25 to 50 nucleotides, wherein the percentage of sequence identity is calculated by comparing the reference sequence to the polynucleotide sequence that may include deletions or addition which total 20 percent or less of the reference sequence over the window of comparison.

“Substantial identity” as applied to polypeptides herein means that two peptide sequences, when optimally aligned, such as by the programs GAP or BESTFIT using default gap weights, share at least 30 percent sequence identity, preferably at least 40 percent sequence identity, and more preferably at least 50 percent sequence identity, and most preferably at lest 60 percent sequence identity. Preferably, residue positions, which are not identical, differ by conservative amino acid substitutions.

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chan is cystein and methionine. Preferred conservative amino acid substitution groups are: valine-leucine-isoleucine; phenylalanine-tyrosine; lysine-arginine; alanine-valine; glutamic-aspartic; and asparagine-glutamine.

“Modulation” refers to the capacity to either enhance or inhibit a functional property of a biological activity or process, for example, enzyme activity or receptor binding. Such enhancement or inhibition may be contingent on the occurrence of a specific event, such as activation of a signal transduction pathway and/or may be manifest only in particular cell types.

“Modulator” refers to a chemical (naturally occurring or non-naturally occurring), such as a biological macromolecule (for example, nucleic acid, protein, non-peptide or organic molecule) or an extract made from biological materials, such as prokaryotes, bacteria, eukaryotes, plants, fungi, multicellular organisms or animals, invertebrates, vertebrates, mammals and humans, including, where appropriate, extracts of: whole organisms or portions of organisms, cells, organs, tissues, fluids, whole cultures or portions of cultures, or environmental samples or portions thereof. Modulators are typically evaluated for potential activity as inhibitors or activators (directly or indirectly) of a biological process or processes (for example, agonist, partial antagonist, partial agonist, antagonist, antineoplastic, cytotoxic, inhibitors of neoplastic transformation or cell proliferation, cell proliferation promoting agents, antiviral agents, antimicrobial agents, antibacterial agents, antibiotics, and the like) by inclusion in assays described herein. The activity of a modulator may be known, unknown or partially known.

“Test chemical” refers to a chemical or extract to be tested by at least one method of the present invention to be a putative modulator. A test chemical is usually not known to bind to the target of interest. “Control test chemical” refers to a chemical known to bind to the target (for example, a known agonist, antagonist, partial agonist or inverse agonist). Test chemical does not typically include a chemical added to a mixture as a control condition that alters the function of the target to determine signal specificity in an assay. Such control chemicals or conditions include chemicals that (1) non-specifically or substantially disrupt protein structure (for example denaturing agents such as urea or guandium, sulfhydryl reagents such as dithiotritol and beta-mercaptoethanol), (2) generally inhibit cell metabolism (for example mitochondrial uncouples) and (3) non-specifically disrupt electrostatic or hydrophobic interactions of a protein (for example, high salt concentrations or detergents at concentrations sufficient to non-specifically disrupt hydrophobic or electrostatic interactions). The term test chemical also does not typically include chemicals known to be unsuitable for a therapeutic use for a particular indication due to toxicity of the subject. Usually, various predetermined concentrations of test chemicals are used for determining their activity. If the molecular weight of a test chemical is known, the following ranges of concentrations can be used: between about 0.001 micromolar and about 10 millimolar, preferably between about 0.01 micromolar and about 1 millimolar, more preferably between about 0.1 micromolar and about 100 micromolar. When extracts are uses a test chemicals, the concentration of test chemical used can be expressed on a weight to volume basis. Under these circumstances, the following ranges of concentrations can be used: between about 0.001 micrograms/ml and about 100 milligram/ml, preferably between about 0.01 micrograms/ml and about 10 milligrams/ml, and more preferably between about 0.1 micrograms/ml and about 1 milligrams/ml or between about 1 microgram/ml and about 100 micrograms/ml.

“Target” refers to a biochemical entity involved in a biological process. Targets are typically proteins that play a useful role in the physiology or biology of an organism. A therapeutic chemical typically binds to a target to alter or modulate its function. As used herein, targets can include, but not be limited to, cell surface receptors, G-proteins, G-protein coupled receptors, kinases, phosphatases, ion channels, lipases, phosholipases, nuclear receptors, intracellular structures, tubules, tubulin, and the like.

“Label” or “labeled” refers to incorporation of a detectable marker, for example by incorporation of a radiolabled compound or attachment to a polypeptide of moieties such as biotin that can be detected by the binding of a section moiety, such as marked avidin. Various methods of labeling polypeptide, nucleic acids, carbohydrates, and other biological or organic molecules are known in the art. Such labels can have a variety of readouts, such as radioactivity, fluorescence, color, chemiluminescence or other readouts known in the art or later developed. The readouts can be based on enzymatic activity, such as beta-galactosidase, beta-lactamase, horseradish peroxidase, alkaline phosphatase, luciferase; radioisotopes such as ³H, ¹⁴C, ³⁵S, ¹²⁵I or ¹³¹I); fluorescent proteins, such as green fluorescent proteins; or other fluorescent labels, such as FITC, rhodamine, and lanthanides. Where appropriate, these labels can be the product of the expression of reporter genes, as that term is understood in the art. Examples of reporter genes are beta-lactamase (U.S. Pat. No. 5,741,657 to Tsien et al., issued Apr. 21, 1998) and green fluorescent protein (U.S. Pat. No. 5,777,079 to Tsien et al., issued Jul. 7, 1998; U.S. Pat. No. 5,804,387 to Cormack et al., issued Sep. 8, 1998).

“Substantially pure” refers to an object species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, as substantially pure composition will comprise more than about 80 percent of all macromolecular species or activities present in a composition, more preferably more than about 85%, 90%, 95% and 99%. Most preferably, the object species or activity is purified to essential homogeneity, wherein contaminant species or activities cannot be detected by conventional detection methods) wherein the composition consists essentially of a single macromolecular species or activity. The inventors recognize that an activity may be caused, directly or indirectly, by a single species or a plurality of species within a composition, particularly with extracts.

A “PKS activity” is at least one activity of at least one PKS, such as, for example, an aromatic PKS system, a modular PKS system or a fungal PKS system.

An “aromatic PKS system” refers to a PKS system characterized by the iterative use of the catalytic sites on the several enzymes produced. Thus, in aromatic PKS systems, only one enzyme with a specific type of activity is produced to catalyze the relevant activity for the system throughout the synthesis of the polyketide. In aromatic PKS systems, the enzymes of the minimal PKS are encoded in separate open reading frames (ORFs). The actinorhodin PKS is encoded in six separate ORFs. For the minimal PKS, one ORF contains a ketosynthase (KS) and an acyultransferase (AT); a second ORF contains what is believed to be a chain-length factor (CLF); and a third reading frame encodes an acyl carrier protein (ACP). Additional ORFs encode an aromatase (ARO), a cyclase (CYC), and a ketoreductase (KR). The combination of a KS/AT, ACP, and CLF constitutes a minimal PKS, since these elements provide for a single condensation of a two-carbon unit. Furthermore, the gris PKS contains five separate ORFs wherein the KS/AT, CLF, and ACP are on three ORFs, the KR is on a fourth, and the ARO is on a fifth (WO 98/27203 to Barr et al., published Jun. 25, 1998).

A “modular PKS system” refers to a PKS system where each catalytic site is used only once and the entire PKS is encoded as a series of modules. Thus, the modular synthase protein contains a multiplicity of catalytic sites having the same type of catalytic activity. A minimal module contains at least a KS, an AT and an ACP. Optional additional activities include KR, DH, and enoylreductase (ER) and a thoesterast (TE) activity.

A “fungal PKS” encoding a 6-methyl salicylic acid synthase (6-MSAS) has some similarity to both the aromatic and modular PKS. It has only one reading frame for KS, AT, a dehydratase (DH), KR and ACP. Thus, it appears similar to a single module of a modular PKS. Unlike an aromatic PKS, it does not include a CLF.

“Pharmaceutical agent or drug” refers to a chemical, composition or activity capable of inducing a desired therapeutic effect when property administered by an appropriate dose, regime, route of administration, time and delivery modality.

“Pharmaceutical agent or drug” refers to a chemical, composition or activity capable of inducing a desired therapeutic effect when property administered by an appropriate dose, regime, route of administration, time and delivery modality.

A “bioactive compound” refers to a compound that exhibits at least one bioactivity.

A “bioactivity” refers to a composition that exhibits at least one activity that modulates a biological process. Preferred bioactivities include, but are not limited to: antibacterial activity, antimicrobial activity, not being substantially susceptible to multi-drug resistance, antiviral activity, antitumor activity, anticancer cell activity, immunomodulatory activity, anti-inflammatory activity, radiation protective activity, modulating a protein kinase C (PKC) activity or other kinase activity and cytotoxic activity.

“A bryostatin” or “bryostatin” or “bryostatins” refers to a compound that includes a bryopyran ring and has at least one bioactivity.

“Made at least in part” refers to a bioactive compound or bioactivity whose bioactivity derives at least in part from an activity of an entity, such as a marine organism.

A “bioactive derivative” refers to a modification of a bioactive compound or bioactivity that retains at least one characteristic activity of the parent compound.

A “bioactive precursor” refers to a precursor of a bioactive compound or bioactivity that exhibits at least one characteristic activity of the resulting bioactive compound or bioactivity.

An “antibacterial activity” refers to an activity that reduces the growth rate or numbers of living bacteria in a sample, such as a culture of bacteria or a sample that includes at least one bacteria, including a patient. Such antibacterial activity can be directed against Gram-negative and Gram-positive bacteria and can be screened for or confirmed using methods known in the art.

An “antimicrobial activity” refers to an activity that reduces the growth rate or numbers of living microbes in a sample (including prokaryotic and/or eukaryotic microbes), such as a culture of microbes or a sample that includes at least one microbe, including a patient and can be screened for or confirmed using methods known in the art.

“Not substantially susceptible to multiple drug resistance” refers to cells that exhibit multiple drug resistance, such as a against methicillin, vancomycin, bryostatin or taxol, cannot survive or propagate at their usual rate in the presence of a bioactive compound. Such an activity can be confirmed using methods known in the art.

An “antiviral activity” refers to an activity that reduces the infectivity of virus particles in a sample, such as in a sample including at least one virus, including a patient. Such antiviral activity can be directed against, for example, DNA or RNA containing viruses, including, but not limited to herpesvirus, hepatitis virus and retrovirus. Such activity can be screened for using methods known in the art.

An “antitumor activity” refers to an activity that reduces the growth rate or number of tumor cells in a sample, such as a culture of tumor cells or a sample that includes at least one tumor cell, including a patient. Such antitumor activity can be directed against any type of tumor or tumor cell, including, but not limited to renal tumor, lung tumor, colon tumor, central nervous system tumor, melanoma, ovarian tumor and breast tumor.

An “anticancer cell activity” refers to an activity that reduces the growth rate or number of cancer cells in a sample, such as a culture of cancer cells or a sample that includes at least one cancer cell, including a patient. Such anticancer cell activity can be directed against any type of cancer cell, including, but not limited to renal cancer, leukemia, lung cancer, colon cancer, central nervous system cancer, melanoma, ovarian cancer and breast cancer.

An “immunomodulatory activity” refers to an activity that can modulate either or both of the cellular or humoral branch of the immune system of a subject. For example, the modulation, increase or decrease of the activity of the cellular immune response, humoral immune response, or both, can be measured using methods known in the art.

An “anti-inflammatory activity” refers to an activity that reduces the severity or occurrence of an inflamation response. Such activity can be screened using methods known in the art.

A “radiation protective activity” refers to an activity that reduces the severity or occurrence of cellular damage or mutation due to exposure to radiation. Such activity can be screened using methods known in the art.

“Modulate PKC activity” refers to the ability of a compound to bind or modulate at least one activity of at least one PKC. Preferably, the modulation results from the binding of a compound to a PKC.

A “cytotoxic activity” refers to an activity that reduces the number of viable cells in a sample, including prokaryotic cells, eukaryotic cells or both. Such activity can be screened using methods known in the art.

A “patient” or “subject” refers a whole organism in need of treatment, such as a farm animal, companion animal or human. An animal refers to any non-human animal.

Other technical terms used herein have their ordinary meaning in the art that they are used, as exemplified by a variety of technical dictionaries, such as the McGraw-Hill Dictionary of Chemical Terms and the Stedman's Medical Dictionary.

Introduction

The present invention recognizes that marine organisms comprise nucleic acid molecules that encode polypeptides that catalyze the synthesis of bioactive compounds, such as polyketides including bryopyran rings, such as bryostatins.

Polyketide synthase (PKS) genes expected to encode polypeptides necessary to synthesize bryostatins are provided, along with methods for identifying and isolating the PKS genes needed to recombinantly biosynthesize related polyketide molecules through combinatorial synthesis. The cloned genes can also be used to screen environmental samples for novel PKS genes. The cloned genes and linked genes involved in bryostatin synthesis may be transformed and expressed in a desired host organism to produce bryostatins or derivatives thereof for a variety of purposes, including anti-cancer compounds, immunomodulatory compounds, anti-microbial, and anti-fungal compounds.

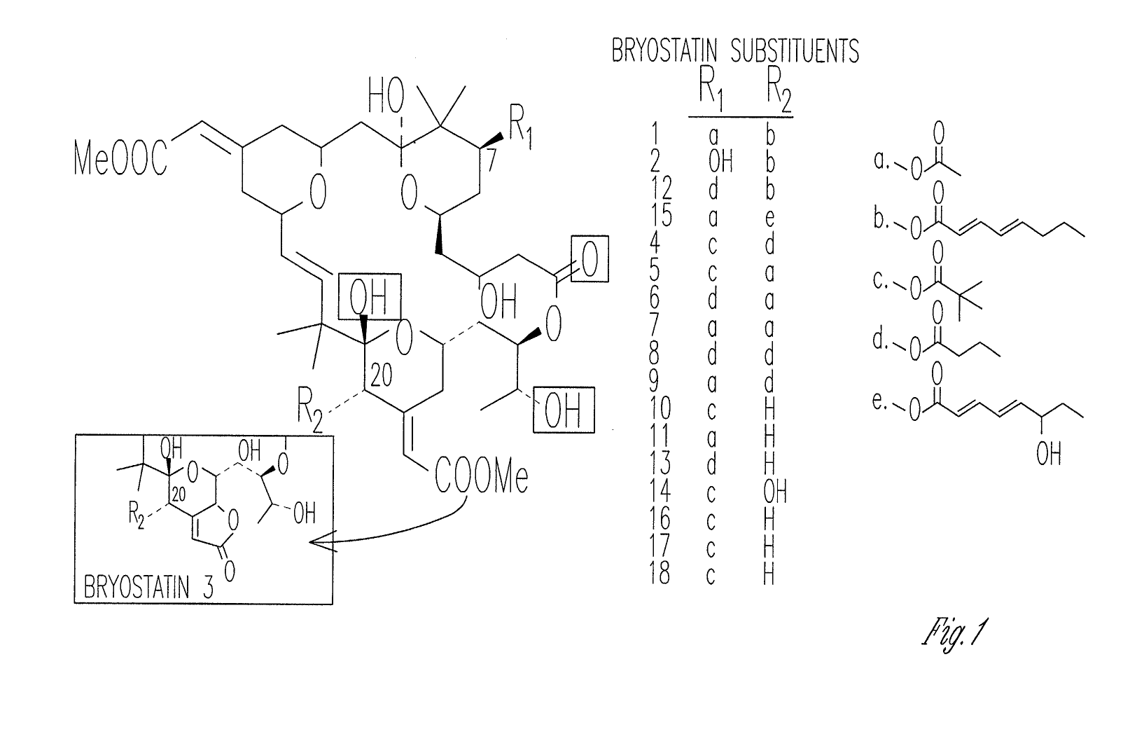

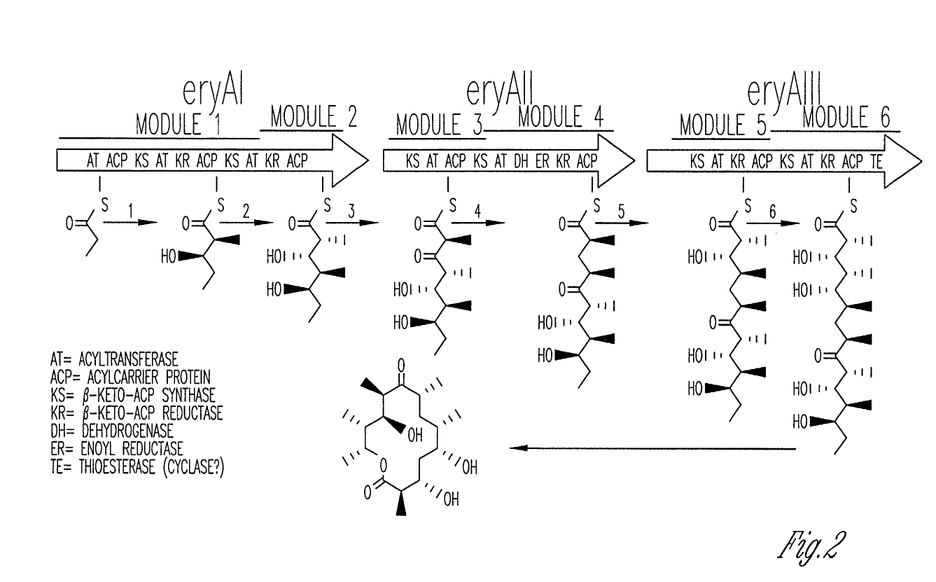

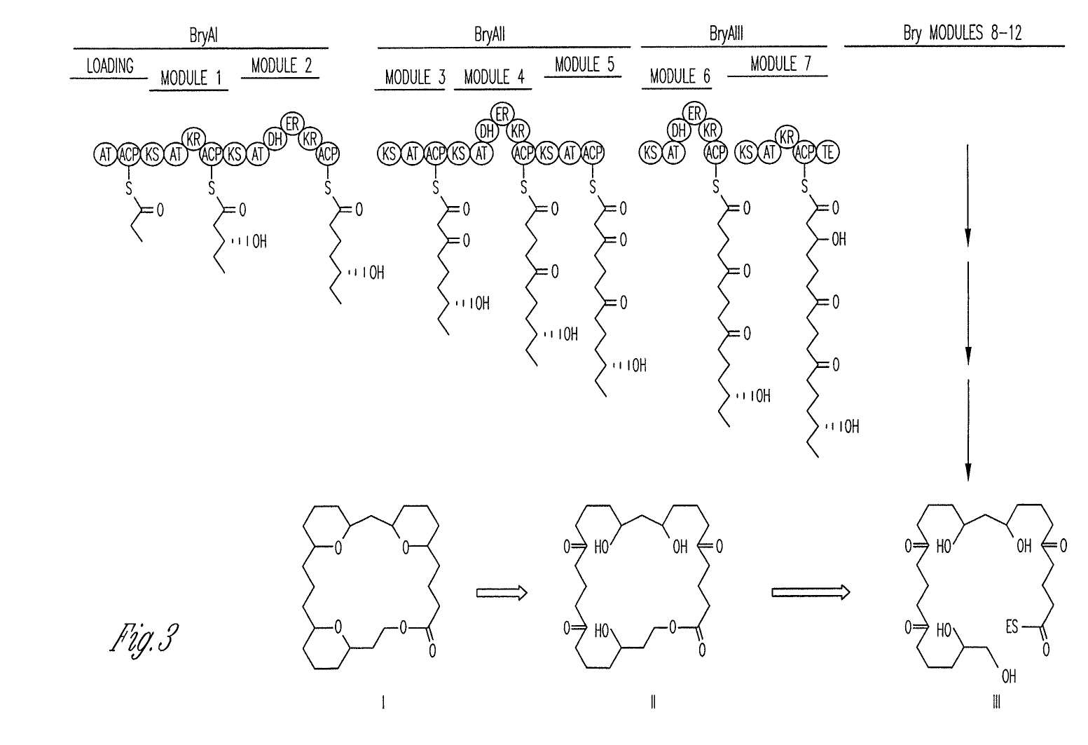

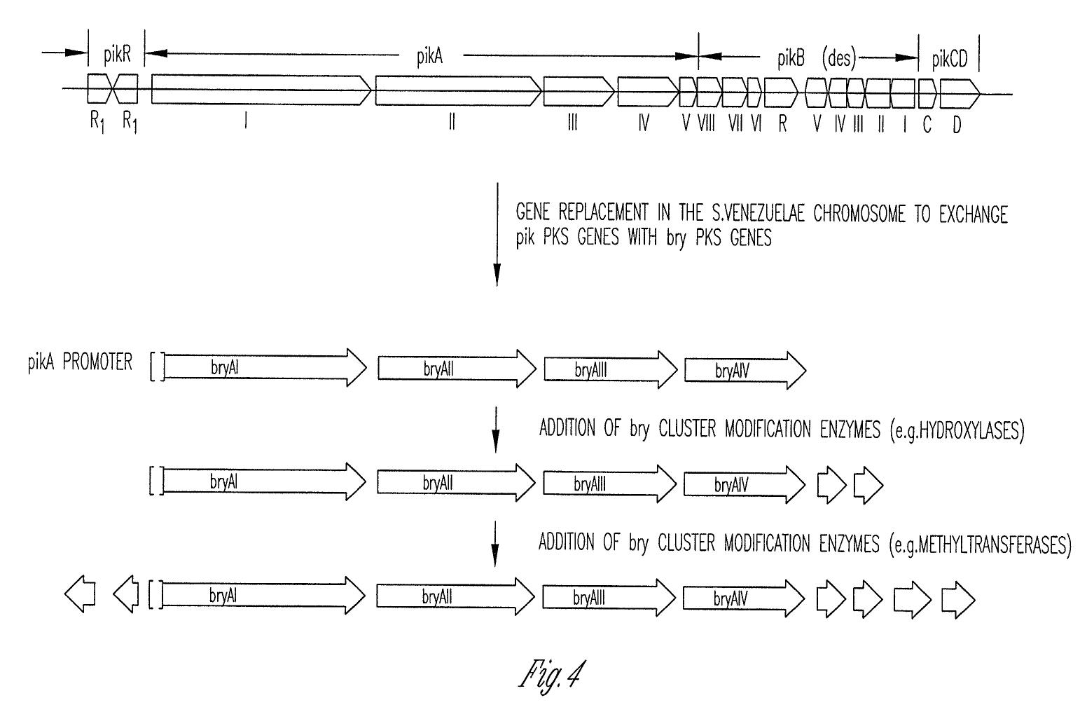

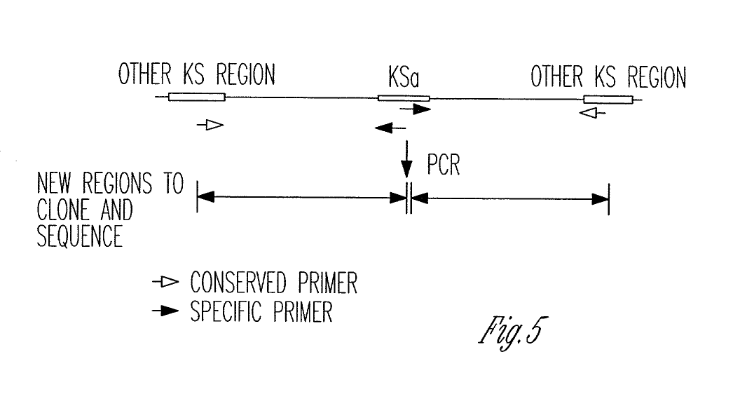

Bryostatins are a unique family of cytotoxic macrolides based on the bryopyran ring system (Pettit, 1991) (FIG. 1). They occur exclusively in the marine bryozoan Bugula neritina. Bryostatin 1 is now in Phase II clinical trials for the treatment of leukemias, lymphomas, melanoma and solid tumors (Pluda et al., 1996). Bryostatin 1 also shows promise for treatment of ovarian and breast cancer and to enhance lymphocyte survival during radiation treatment (Kraft, 1993); (Lind et al., 1993); (Grant et al., 1994); (Scheid et al., 1994); (Sung et al., 1994); (Correale et al., 1995); (Fleming et al., 1995); (Baldwin et al., 1997); (Lipshy et al., 1997); (Taylor et al., 1997); (Basu, 1998); (Johnson et al., 1999). Other bryostatins may ultimately prove even more valuable. In addition, this structure offers exciting possibilities for combinatorial biosynthesis. The cloned genes can be used for combinatorial creation of novel polyketide/bryostatin analogs that may exhibit improved anti-cancer properties. Bryostatins are complex polyketides similar to bacterial secondary metabolites biosynthesized by modular Type I polyketide synthases (PKS-I). Research and development of the bryostatins is currently severely limited by inadequate availability of bryostatins. B. neritina is harbors an uncultivated symbiont, the gamma proteobacterium Candidatus Endobugula sertula. Currently, B. neritina is the exclusive source of the bryostatins.

Unlike most chemotherapeutic agents that kill rapidly dividing cells, bryostatins act on signal transduction pathways by binding to the activator site of protein kinase C (PKC) (Steube and Drexler, 1993); (Caponigro et al., 1997). Eighteen bryostatins have been described (Pettit et al., 1982); (Pettit, 1991); (Pettit et al., 1996). These vary primarily in the substituents at C-7 and C-20.

The major obstacle in investigating and developing bryostatins as anti-cancer agents or for other therapeutic purposes is the difficulty of obtaining them in ample quantities. The yield of bryostatin 1 is low; in the large-scale isolation for clinical trials it was 1.4 micrograms per gram wet weight of B. neritina. (Schaufelberger et al., 1991). The supply of B. neritina is unpredictable and harvesting has long-term negative effects on colonies. Research has focused on bryostatin 1, but the other bryostatins have been isolated on the basis of their antileukemic activity. With the exception of bryostatins 16 and 17, all possess the structural features believed to account for the activity of bryostatin 1 (Pettit et al., 1982); (Pettit, 1991); (Pettit et al., 1991); (Pettit et al., 1996). Other bryostatins may equal or exceed the therapeutic value of bryostatin 1. One study showed that bryostatins 5 and 8 are as effective as bryostatin 1 in treating melanoma, but with milder side effects (Kraft et al., 1996). The unusual biological activities of bryostatins suggest that novel structures based on the bryopyran ring will likely have useful properties as potential drugs. Greater availability of bryostatins is essential to permit research to unlock the potential of this remarkable family of compounds. Although aquaculture would provide a more consistent source of bryostatins, it does not improve the low yield. Large-scale chemical synthesis of bryostatins is currently considered impractical due to their structural complexity (Kageyama et al., 1990); (Wender et al., 1998).

All polyketide biosynthetic systems for bacterial macrolide compounds, that have been studied to date, are similar to the type I fatty acid synthase (FAS) in that they use large multifunctional polypeptides (Schupp et al., 1995). Hence the term type I PKS (PKS-I).

Complex polyketides, like the bryostatins, are typically synthesized by bacteria. B. neritina has a specific bacterial symbiont “Candidatus Endobugula sertula” (Haygood and Davidson, 1997) that the data below suggests can be the true source of the bryostatins. Cloning and expressing the bryostatin genes in a heterologous systems, or cultivating E. sertula (or another B. neritina-associated bacteria which is the source of bryostatin), would prevent a supply problem. In addition, the genes involved in bryostatin synthesis could be combined with other polyketide synthase genes to result in the creation of novel drugs and drug analogs with improved activities.

The majority of pharmaceuticals used in the treatment of breast and other cancers are cytotoxic or cytostatic inhibitors of tumor growth. Despite the use of this type of drug, along with surgery and radiotherapy, in the treatment of the disease, the breast cancer death rate has not decreased (Dickson et al., 1996). This can be attributed to many factors including rising incidence, resistance to therapy, and metastasis of the disease. Since distant metastasis of breast cancer is only indirectly related to tumor size, a logical approach would be to discover drugs which directly interfere with the complex process of metastasis (Dickson et al., 1996). The drug discussed in this patent proposal, bryostatin 1, has shown among other anti-cancer activities, antimetastatic activity (Dickson et al., 1996); (Johnson et al., 1999).

Metastasis of breast cancer involves a multistep process of coordinated gene expression by tumor cells. The progression from primary tumor to metastasis involves a number of malignant characteristics including altered cell-cell and cell-substratum adhesion, increased motility, elaboration of proteases, altered growth control and the ability to produce angiogenic factors (Liotta and Kohn, 1990); (Johnson et al., 1999). These processes are believed to be modulated to some extent by the central signaling pathways of cells. Many of the above properties have been shown in various model systems to be regulated by protein kinase C (PKC)-mediated pathways; agents that modulate PKC have been shown to alter the rate of metastasis in some animal models (Johnson et al., 1999). Increased protein kinase C (PKC) activity in malignant breast tissue and positive correlations between PKC activity and expression of a more aggressive phenotype in breast cancer cell lines, suggest a role for this signal transduction pathway in the pathogenesis and/or progression of breast cancer. Thus, findings suggest that the PKC pathway may modulate progression of breast cancer to a more aggressive neoplastic process (Ways et al., 1995). Numerous studies suggest the use of PKC modulators, such as bryostatin 1, as anti-invasive and/or antimetastatic agents in the treatment of breast cancer (Johnson et al., 1999).

Recently, an exciting potential therapeutic use of PKC modulators has emerged. PKC modulators can interact with many chemotherapeutic agents and potentiate their activity (Caponigro et al., 1997). PKC isozymes have been implicated in the regulation of the multidrug resistance (MDR) phenotype. The MDR phenotype is expressed by some tumor cell populations, in which a drug efflux pump is activated with consequent cross-resistance to major classes of anticancer drugs in clinical use (vinca alkaloids, anthracyclines, podophillotoxins, taxanes) due to reduced intracellular drug accumulation (Korczak et al., 1989); (Caponigro et al., 1997). The MDR phenotype is accompanied by changes in the PKC activity and many observations indicate a role for PKC in the regulation of this phenotype (Fine et al., 1988); (Caponigro et al., 1997). There is preclinical evidence of antiproliferative activity of PKC modulators (Johnson et al., 1999). In addition, encouraging results have been obtained in combined administration of PKC modulators and other cytotoxic drugs, including those involved in the MDR phenotype (Caponigro et al., 1997). In contrast to the often severe effects of other MDR reversal agents, PKC modulators appear to act through a different mechanism. In the only clinical trail of a drug belonging to this class, used in combination with doxorubicin, serum levels approximating those that potentiate the effects of chemotherapy in tumor-bearing animals were achieved without significant toxicity, while no pharmokinetic interaction has been recorded (Jayson et al., 1995). Thus, drugs such as bryostatin 1 targeting PKC may be useful as a means of counteracting drug resistance during cancer chemotherapy.

Studies also indicate that bryostatin 1 may be an effective cancer treatment when combined with other drugs. For example, bryostatin 1 in combination with IL-2 in vitro enhances proliferation and IL-2 receptor expression on lymphocytes, favoring CD8+ cells while suppressing the generation of lymphokine-activated killer (LAK) activity (Scheid et al., 1994). In cancer patients, intravenous administration of bryostatin 1 increased the potential of IL-2 to induce proliferation of LAK activity in lymphocytes, which the authors suggest makes bryostatin an interesting candidate for clinical trials in combination with IL-2 (Scheid et al., 1994). With respect to breast cancer, a recent study suggested that bryostatin 1 sensitized human breast cancer cells to the cytotoxic effects of gemcitabine (Philip et al., 1999).

Thus, bryostatin 1 and analogs thereof derived from combinatorial biosynthesis are excellent candidates for the treatment of breast cancer. Bryostatin 1 has tremendous potential as a breast cancer treatment based on antineoplastic activity, antimetastatic activity and immunostimulation during chemotherapy. Bryostatin has even greater potential in combination therapy as adjuncts to known anticancer agents against the MDR phenotype. However, large-scale clinical studies and ultimate supply of bryostatin will be hampered by a supply problem, as was the case for taxol. The application of this patent (ie. cloning and expressing the bryostatin biosynthesis genes) could avoid this problem. Furthermore, the cloning of the bryostatin biosynthesis genes could lead to combinatorial biosynthesis of bryostatin analogs exhibiting improved anti-cancer properties.

The structure of bryostatins and biosynthesis thereof suggest that it is synthesized by a Type I polyketide synthase (PKS-I). The cloning and expression of this polyketide synthase and associated tailoring enzymes from B. neritina would allow production of essentially unlimited amounts of bryostatins. In addition, the structure of bryostatin offers exciting possibilities for combinatorial biosynthesis of a wide variety of compounds, including novel compounds. The cloned genes can used for combinatorial creation of novel bryostatin analogs that may exhibit improved properties.

As a non-limiting introduction to the breath of the present invention, the present invention includes several general and useful aspects, including:

1) a composition including at least one nucleic acid molecule that encodes at least one polypeptide that catalyzes at least one step in the synthesis of at least one polyketide such as a bryopyran ring, wherein said at least one nucleic acid is derived from at least one marine organism;

2) a composition including a library of nucleic acid molecules of 1);

3) a composition including at least one polypeptide that catalyzes at least one step in the synthesis of at least one polyketide such as a bryopyran ring, wherein said at least one polypeptide is derived from at least one marine organism;

4) a composition including a library of polypeptides of 3);

5) a method of making a composition including providing at least one composition of 1), and synthesizing at least one polyketide such as a bryopyran ring;

6) a composition made by the method of 5);

7) a method of making a composition including providing at least one composition of 3), and synthesizing at least one bryopyran ring;

8) a composition made by the method of 7);

9) a method for identifying at least one nucleic acid molecule encoding at least one activity of a PKS including contacting a nucleic acid molecule of 1) with a sample, and identifying nucleic acid molecules in said sample that hybridize with said nucleic acid molecule of 1);

10) a nucleic acid molecule identified by the method of 9);

11) a composition comprising a library of nucleic acid molecules of 10);

12) a method for identifying a bioactive compound including contacting a composition of 5) and determining the bioactivity of said compound;

13) a method for identifying a bioactive compound including contacting a composition of 8) and determining the bioactivity of said compound;

14) a preparation of bacteria from a Bugula that include PKS genes; and

15) a composition comprising at least one polyketide, bryopyran ring or bryostatin present in Bugula pacifica.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

I. Nucleic Acid Molecules That Encode Polypeptides That Catalyzes the Synthesis of Polyketides Such as Bryopyran Rings and Libraries of Such Nucleic Acid Molecules

The present invention includes a composition including at least one nucleic acid molecule, such as a substantially purified or purified nucleic acid molecule, that encodes at least a portion of at least one polypeptide that catalyzes at least one step in the synthesis of at least one polyketide such as a bryopyran ring, such as a bryostatin. Preferably, at least one nucleic acid molecule is derived from at least one marine organism. The nucleic acid molecules of the present invention can comprise the nucleic acid molecules disclosed herein, including PCR primers, portions thereof, and nucleic acid molecules that selectively hybridize with or have substantial identity with the nucleic acid molecules disclosed herein or portions thereof, or encode at least one conservative amino acid substitution relative to the disclosed sequences or portions thereof. A nucleic acid molecule of the present invention can be DNA, RNA, single stranded, double stranded or any combination thereof.

A nucleic acid molecule of the present invention preferably encodes at least a portion of a polypeptide involved in the synthesis of at least one polyketide. Preferably, the polypeptide is at least a portion of a polyketide synthase, including PKS type I or PKS type II enzymes. Preferably, the polyketide synthase is a PKS type I enzyme, which can include a plurality of active domains that are involved in the synthesis of a polyketide, such as a bryopyran ring. A nucleic acid molecule of the present invention preferably encodes at least a portion of at least one such active domain and can include at least one activity of such an active domain, preferably an activity that catalyzes at least one step in the synthesis of a polyketide. Preferably, a nucleic acid molecule of the present invention is between about 1 Kb and about 100 Kb, between about 5 Kb and about 50 Kb or between about 10 Kb and about 25 Kb in length and about 100 Kb in length.

A nucleic acid molecule of the present invention can be derived from at least one marine organism. A marine organism can include any organism that can be found in a marine environment, either naturally or xenotypically. A marine organism can be a vertebrate, an invertebrate or a unicellular organism, such as a fungi, algae or bacteria. Preferably, a marine organism is an invertebrate, such as a Bugula, such as Bugula neritina or Bugula pacifica, or a unicellular organism, such as a bacteria, such as an Endobugula, such as an Endobugula sertula.

A nucleic acid molecule of the present invention from a marine organism can be characterized as having an unusually low G:C content, for example between about 35% and about 55%, which can vary depending on the particular marine organism. Certain nucleic acid molecules of the present invention exemplified in SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 and SEQ ID NO:29 through SEQ ID NO:37 or portions thereof or nucleic acid molecules including at least a portion thereof have a G:C content ranging from about 35% to about 55%. This low G:C content is particularly noted in symbionts of Bugula neritina and Bugula pacifica.

A nucleic acid of the present invention can also encode a fusion protein that includes a polypeptide of the present invention and a polypeptide of interest. A polypeptide of interest can be any polypeptide, but is preferably a detectable label, such as green fluorescent protein, or a sequence that aids in the purification of a polypeptide, such as FLAG. A nucleic acid that encodes a fusion protein can be made by operably linking a nucleic acid that encodes a polypeptide of interest with a polypeptide of the present invention. The operably linking can be direct or indirect, such as in the case where a linker connects the polypeptide of the present invention with a polypeptide of interest. The nucleic acid molecule of the present invention and the nucleic acid that encodes a polypeptide of interest are preferably operably linked in frame such that an operable polypeptide of the present invention and an operable polypeptide of interest are translated from the nucleic acid, but that need not be the case.

Nucleic acid molecules of the present invention can be made using methods known in the art and described herein (see, Sambrook et al., supra (1989)). For example, nucleic acid molecules of the present invention can be identified and isolated using PCR methodologies, including RT-PCR, and sequenced using established methods such that their homologies can be determined. The ability of one nucleic acid molecule to hybridize with another can be determined through experimentation under a variety of stringencies, or can be estimated based on their length and G:C contents. Alterations of identified sequences can be made using routine methods, such as mutagenesis, RT-PCR or other PCR methods (See, Sambrook et al, supra, (1989)).

A nucleic acid molecule of the present invention can include at least one expression control sequence. Preferably, an expression control sequence is operably linked to a nucleic acid molecule such that the nucleic acid molecule can be expressed in an in vivo or in vitro transcription and/or translation system. The choice of expression control sequences is dependent upon the transcription system to be used. For example, if a prokaryotic organism such as E. coli is to be used to express a nucleic acid molecule, then at least one appropriate prokaryotic expression control sequence would be used. Likewise, if a eukaryotic organism is to be used to express a nucleic acid molecule, then at least one appropriate eukaryotic expression control sequence, such as CMV or LTRs would be used. Such nucleic acid molecules can be in any form, such as in a plasmid or in a linear form.

A nucleic acid molecule of the present invention can be provided with or without expression control sequences in a vector, such as a plasmid or a viral vector. Viral vectors can be chosen so that they are appropriate for a cell to be transfected, such as, for example, a phage, cosmid, retrovirus, vaccinia, adenovirus or adenoassociated virus. Viral vectors can introduce a nucleic acid molecule into a cell during its normal biological processes. Non-viral vectors can be used to introduce a nucleic acid molecule of the present invention into a host cell using methods known in the art, such as lipofection, cold calcium chloride or electroporation. The nucleic acid molecule in a cell can be extrachromosomal or be integrated into the genome of the cell. The host cell can be any appropriate host cell, such as a eukaryotic or prokaryotic cell. Preferably, a nucleic acid molecule of the present invention is expressed in the cell, but that is not a requirement of the invention. Preferably, the cell does not normally include a nucleic acid molecule of the present invention or express a polypeptide of the present invention, but that need not be the case. For example, a cell that expresses a relatively low amount of a polypeptide of the present invention can be made to express relatively higher amounts of a polypeptide once transfected with a nucleic acid of the present invention.

Cells that express a polypeptide of the present invention can be screened for and selected using a variety of methods, including those set forth in the present invention. For example, immunoassays, such as western blots, can be used to identify cell lysates that include a polypeptide of the present invention. In addition, immunocytochemistry can be used can be used to identify and localize a polypeptide of the present invention on or within a cell. Furthermore, in situ hybridization methods, such as FISH, can be used to identify and localize nucleic acid molecules within a cell and hybridization methods can be used to identify nucleic acid molecules, either DNA or RNA for cellular preparations. Cells or cell lysates can be screened for an activity using a variety of methods. For example, the ability of a cell or cell lysate to bind with a substrate or convert a substrate, including a detectably labeled substrate, can be used to detect a particular activity (see Haygood and Davidson, 1997).

As set forth in the Examples and exemplified in SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 and SEQ ID NO:29 through SEQ ID NO:37 or portions thereof or nucleic acid molecules including at least a portion thereof, nucleic acid molecules of the present invention can encode peptides that encode PKS activity. Nucleic acid molecules having PKS activity, or other activities associated with PKS, can be identified by making comparisons of nucleic acid sequence or translation amino acid sequences derived therefrom using methods known in the art, including BLAST comparisons. A nucleic acid molecule of the present invention can be expressed and the expression products screened and confirmed for having PKS activity (see, McDaniel et al., 1999; Shen et al. 1999). In addition, nucleic acid molecules of the present invention can encode polypeptides that have other activities of PKS. Methods for screening such activities are known in the art (see, McDaniel et al., 1999).

The present invention also includes a library of nucleic acids of the present invention. A library of nucleic acids includes between about two, about four, about six, about eight, about ten, about thirty, about seventy, about one-hundred, about one-thousand, about ten-thousand, about one-hundred thousand or about one-million nucleic acid molecules and about three, about five, about seven, about twenty, about fifty, about five-hundred, about fifty-thousand, about five-hundred thousand and about ten million nucleic acid molecules. The members of such a library are preferably different nucleic acid molecules, but that need not be the case.

The nucleic acid molecules of the present invention can be used for a variety of applications, including but not limited to, PCR primers, probes to identify similar sequences, and to make polypeptides of the present invention. The particular application of a nucleic acid molecule depends on a variety of factors, such as they are known in the art, include the length, strandedness (single stranded or double stranded and positive sense or negative sense), chemical characterization (such as DNA or RNA) or whether the nucleic acid molecule is detectably labeled or not.

II. A Polypeptide That Catalyzes the Synthesis of Polyketides Such as Bryopyran Rings and Libraries Thereof

The present invention also includes a composition including at least one polypeptide or a portion thereof that catalyzes at least one step in the synthesis of at least one polyketide bryopyran ring, wherein the at least one polypeptide or a portion thereof is derived from at least one marine organism.

A polypeptide of the present invention can be derived from at least one marine organism. A marine organism can include any organism that can be found in a marine environment, either naturally or xenotypically. A marine organism can be a vertebrate, an invertebrate or a unicellular organism, such as a fungi, algae or bacteria. Preferably, a marine organism is an invertebrate, such as a Bugula, such as Bugula neritina or Bugula pacifica, or a unicellular organism, such as a bacteria, such as a Candidatus, such as an Endobugula, such as an Endobugula sertula.

The nucleic acid molecules of the present invention can be translated to provide polypeptides. These polypeptides can be substantially purified or purified and preferably have at least one activity of a polyketide synthase, such as a PKS type I or a PKS type II, including a PKS that is involved in the synthesis of a bryopyran ring, including a bryostatin. The activity of the polypeptide can be screened and confirmed using methods known in the art, later developed or described herein. For example, antibodies that bind with active portions or fragments of polyketide synthases can be used to identify appropriate polypeptides. Alternatively, substrates for an activity, such as substrates that are detectably labeled, can be used to detect the binding of a substrate to an activity or the conversion of a substrate to a product. As set forth in the Examples and exemplified in SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28 and SEQ ID NO:38 or portions thereof or polypeptides or proteins including at least a portion thereof, polypeptides encoding PKS activity have been isolated. The PK activity of polypeptides of the present invention can be screened and confirmed using methods known in the art (see, McDaniel et al., 1999; Shen et al. 1999).

A polypeptide of the present invention can be of any length, but is preferably between about 10 amino acids and about 300,000 amino acids in length and more preferably between about 100 amino acids and about 30,000 amino acids in length or between about 1,000 amino acids and about 3,000 amino acids in length.

The polypeptide of the present invention can be made using recognized methods, such as by way of recombinant methods as they are known in the art (see, Sambrook et al., supra, (1989)) or by digesting proteins or polypeptides. For example, nucleic acid molecules encoding or suspected of encoding a polypeptide of the present invention can be cloned into expression vectors that are transformed into appropriate host cells where the nucleic acid molecules are expressed. The resulting polypeptides can be optionally purified and their activity confirmed using methods of the present invention or as they are known in the art or later developed. Alternatively, the in vivo activity of polypeptides can be confirmed using methods of the present invention or as they are known in the art.

A polypeptide of the present invention can be provided ex vivo or within a cell. A polypeptide of the present invention can be expressed within a cell by transfecting a cell with a nucleic acid molecule that encodes a polypeptide of the present invention. The nucleic acid molecule of the present invention can be operably linked to expression control sequences appropriate for the cell such that the nucleic acid molecule of the present invention is expressed on or within the cell. The nucleic acid molecule can also encode a fusion protein such that the fusion protein is expressed on or within the cell. In this instance, a fusion protein that includes a detectable label as the polypeptide of interest can be used to track the location of the fusion protein in the cell.

A polypeptide of the present invention can also be part of a fusion protein that includes a polypeptide of the present invention and a polypeptide of interest. A polypeptide of interest can be any polypeptide, but is preferably a detectable label, such as green fluorescent protein, or a sequence that aids in the purification of a polypeptide, such as FLAG. A fusion protein that includes a polypeptide of the present invention can be made from a nucleic acid that encodes a fusion protein can be made by operably linking a nucleic acid that encodes a polypeptide of interest with a polypeptide of the present invention. The operably linking can be direct or indirect, such as in the case where a linker connects the polypeptide of the present invention with a polypeptide of interest. The nucleic acid molecule of the present invention and the nucleic acid that encodes a polypeptide of interest are preferably operably linked in frame such that an operable polypeptide of the present invention and an operable polypeptide of interest are translated from the nucleic acid, but that need not be the case. The present invention also includes such fusion proteins or libraries of such fusion proteins.

The present invention also includes a library of polypeptides of the present invention. A library of polypeptides of the present invention, including fusion proteins, includes between about two, about four, about six, about eight, about ten, about thirty, about seventy, about one-hundred, about one-thousand, about ten-thousand, about one-hundred thousand or about one-million polypeptides and about three, about five, about seven, about twenty, about fifty, about five-hundred, about fifty-thousand, about five-hundred thousand and about ten million polypeptides. The members of such a library are preferably different polypeptides, but that need not be the case.

The present invention also includes antibodies that specifically bind with a polypeptide of the present invention. Such antibodies can be polyclonal or monoclonal and can be made using methods known in the art (see, Harrow, Antibodies: A Laboratory Manual, Cold Spring Harbor Press, 1988). The specificity of such antibodies can be screened and confirmed using assay formats known in the art, such as using enzyme linked immunosorbent assays (ELISAs) or other appropriate immunoassay formats.

III. Method of Making Polyketides Such as Bryopyran Rings Using Nucleic Acids or Polypeptides of the Present Invention and Compositions Made Thereby

The present invention also includes a method of making a composition including providing at least one nucleic acid molecule or polypeptide of the present invention, and synthesizing at least one polyketide or precursor thereof, such as a bryopyran ring, such as a bryostatin.

At least one nucleic acid molecule of the present invention or at least one polypeptide of the present invention can be expressed and used in a system to synthesize a polyketide or precursor thereof, including a bryopyran ring, such as a bryostatin. The polyketides or precursors thereof can be previously known or unknown polyketides or precursors thereof. A variety of methods of producing polyketides or precursors thereof using known PKS genes, in particular known PKS type I genes, have been reported. (see, U.S. Pat. No. 5,672,491 to Khosla et al., issued Sep. 30, 1997; U.S. Pat. No. 5,712,146 to Khosla et al., issued Jan. 27, 1998; U.S. Pat. No. 5,716,849 to Ligon et al., issued Feb. 10, 1998; U.S. Pat. No. 5,744,350 to Vinci et al., issued Apr. 28, 1998; U.S. Pat. No. 5,783,431 to Peterson et al., issued Jul. 21, 1998; U.S. Pat. No. 5,824,513 to Katz et al., issued Oct. 20, 1998; U.S. Pat. No. 5,830,750 to Khosla et al., issued Nov. 3, 1998; U.S. Pat. No. 5,843,718 to Khosla et al., issued Dec. 1, 1998; U.S. Pat. No. 5,849,541 to Vinci et al., issued Dec. 15, 1998; U.S. Pat. No. 5,876,991 to DeHoff et al., issued Mar. 2, 1999; WO 93/13663 to Katz et al., published Jul. 22, 1993; WO 95/12661 to Vinci et al., published May 11, 1995; WO 97/22711 to Sherman et al., published Jun. 26, 1997; WO 98/01546 to Leadlay et al., published Jan. 15, 1998; WO 98/11230 to Bloom et al., published Mar. 19, 1998; WO 98/27203 to Barr et al., published Jun. 25, 1998; WO 98/49315 to Khosla et al., published Nov. 5, 1998; WO 98/53097 to Waters et al., published Nov. 26, 1998; WO 99/02669 to Betlach, published Jan. 21, 1999; EP 791,655 A2 to Dehoff et al., Published Aug. 27, 1997; EP 791,656 A2 to Burgett et al., published Aug. 27, 1997).

The present invention utilizes at least one nucleic acid molecules of the present invention and/or at least one polypeptides of the present invention in such methods to make known or novel polyketides, including bryopyran rings and bryostatins. The present invention can utilize at least one nucleic acid molecule of the present invention and/or at least one polypeptide of the present invention alone or in combination with other PKS polypeptides or PKS genes, such as PKS type I polypeptides or PKS type I genes. These polypeptides and genes can be known or later developed and can be from any type of PKS, including PKS derived from marine, aquatic or terrestrial organisms. For example, a PKS can be an aromatic PKS system, a modular PKS system, a fungal PKS system or modified forms thereof (see, WO 98/27203 to Barr et al., published Jun. 25, 1998).

For example, some methods of synthesizing polyketides provide cassettes that include a PKS type I gene complex, either in whole or in part. Nucleic acid molecules of the present invention can be inserted into such cassettes randomly or non-randomly, including replacing identified PKS type genes. Random integration can be accomplished using the methods of Whitney et al., (WO 98/13353, published Apr. 2, 1998) and non-random integration can be accomplished using the methods of Smith et al., (WO 94/24301, published Oct. 27, 1994). When nucleic acid molecules of the present invention are inserted non-randomly into such cassettes, they can be inserted in-frame to replace PKS genes that encode polypeptides that have functions similar to the polypeptide encoded by a nucleic acid of the present invention. The nucleic acid molecules of the present invention are thus expressed as polypeptides of the present invention, which can act as part of a PKS complex to produce known or novel polyketides, such as bryopyran rings including byrostatins.

Cells or extracts thereof (such as substantially purified extracts) that include one or more of the nucleic acid molecules of the present invention or one or more polypeptides of the present invention can be used to synthesize a wide variety of polyketides, including bryopyran rings and bryostatins. Such cells or extracts thereof can be contacted with a variety of compounds, including substrates for PKS activity, particularly PKS activity present in the cells or extracts thereof. Polypeptides expressed from nucleic acids of the present invention can act on these compounds in order to make a wide variety of polyketides such as bryopyran rings including bryostatins. In one aspect of the present invention, more than one cell and/or extract thereof can be used in combination or sequentially such that the products made by combination of cells or extracts can be determined and its activity confirmed.

The present invention also includes compounds made or identified using the present invention. For example, the present invention includes polyketides, bryopyran rings and bryostatins made using at least one method of the present invention. A compound made or identified using a method of the present invention can be a novel or non-novel compound. For example, a compound of the present invention can optionally not include a compound that was not novel on the date of the filing of the present application, or one year or six months prior to the filing date of the present application. In this aspect of the present invention, the compound of the present invention preferably does not include a known bryostatin.

A compound of the present invention can be provided with at least one pharmaceutically acceptable carrier as they are known in the art and discussed herein. Such pharmaceutically acceptable carriers are known in the art and are disclosed herein. A compound of the present invention can also be a pharmaceutical composition.

IV. Method of Identifying Nucleic Acid Molecules, Nucleic Acid Molecules Identified Thereby and Libraries of Nucleic Acid Molecules

The present invention also includes a method for identifying at least one nucleic acid molecule encoding at least one activity of a PKS including contacting a nucleic acid molecule of the present invention with a sample, and identifying nucleic acid molecules in said sample that hybridize with said nucleic acid molecule of the present invention. This aspect of the present invention utilizes nucleic acid molecules of the present invention as probes or PCR primers in order to identify nucleic acid molecules that have or are expected to encode polypeptides that have PKS activity.

Samples for use in the present invention can be from any source that can include a nucleic acid molecule, but preferably include samples from an environmental sample, such as the marine environment. The samples can include marine organisms, including invertebrates or vertebrates or any other marine organism. Preferably, the sample includes single celled organisms, such as bacteria. More preferably, the sample includes samples that are expected to contain a polyketide, such as a bryopyran including bryostatins. Such samples include, for example, Bugula species, including Bugula neritina and Bugula pacifica.

When used as probes, a nucleic acid molecule of the present invention can be detectably labeled and contacted with a sample. Nucleic acid molecules that bind with the nucleic acid of the present invention can be identified, cloned and sequenced using methods known in the art. The identified nucleic acid molecules can be operably linked to expression control sequences such that a polypeptide encoded by the identified nucleic acid molecule can be made and characterized.

When used as PCR primers, the nucleic acid molecules of the present invention can be used to amplify nucleic molecules in a sample. The amplified nucleic acid molecules are presumptively derived from a PKS gene. The amplified nucleic acid molecules can be identified, cloned and sequenced using methods known in the art. The amplified nucleic acid molecules can be operably linked to expression control sequences such that a polypeptide encoded by the amplified nucleic acid molecule can be made and characterized.

The nucleic acid molecules of the present invention can also be used to identify nucleic acid molecules that are upstream or downstream from a targeted segment of a PKS gene. A nucleic acid molecule that encodes a conserved region of a PKS gene can be used in primer extension or inverse PCR methods such that upstream or downstream segments from the point of hybridization are identified. These extended segments can be identified, cloned and sequenced using methods known in the art. The extended segments can be operably linked to expression control sequences such that a polypeptide encoded by the extended segment can be made and characterized.

Whether the nucleic acid molecule of the present invention is used as a probe, primer or PCR primer, the methods of the present invention identify nucleic acid molecules that presumptively encode at least a portion of a PKS gene. Any of these processes can be used alone, in combination or reiteratively to identify at least portions of PKS genes in a sample.

The present invention also includes nucleic acid molecules identified by the present invention. The identified nucleic acid molecules can include expression control sequences operably linked to the identified nucleic acid molecules. Such constructs can be used to make polypeptides encoded by the identified nucleic acid molecules and the polypeptides can be characterized as to a variety of structures and functions, particularly structures and functions associated with PKS genes. The present invention includes a library of nucleic acids, cells or polypeptides identified by the present invention.

V. Method of Identifying a Bioactive Compound, Bioactive Compounds, and Therapeutic Compositions

The present invention also includes a method for identifying a bioactive compound including contacting a compound made or identified by the present invention with at least one in vitro, ex vivo or in vivo assay system and determining the bioactivity of said compound. The present invention includes bioactive compounds identified using this method. The identified bioactive compounds can be provided in a pharmaceutically acceptable carrier and can be a pharmaceutical compound.

In vitro, ex vivo and in vivo systems used in the present invention are preferably those known in the art for a bioactivity to be identified. The assay chosen to be used in this method is related to a bioactivity that is being screened for. Preferred systems include those that determine at least one PKS activity, such as one activity of a bryopyran ring, including at least one activity of a bryostatin. For example, in vitro systems (systems that do not use whole organisms or whole cells) and ex vivo systems (systems that use whole cells or portions of cells) for the identification of polyketide, bryopyran ring or bryostatin activity are known in the art (see DeVries et al., 1988). In vivo systems (systems that use whole organisms or tissues or organs derived therefrom) are also known in the art (see DeVries et al., supra, 1988). Compounds that are identified using these methods as having a desired bioactivity. Compounds identified by these methods are bioactive compounds that have at least one bioactivity.

Screening of Compounds for Activities

The following assays can be performed to confirm the bioactivity of a compound:

a) antimicrobial effect on S. aureus by placing a compound on a paper disk and determining the ability of the compound to inhibit the growth of the S. aureus (Benson, Microbial Applications, 6^{th} Ed. Wm. C. Brown Publishers, Dubuque, Iowa (1994)). The results of this assay establish the toxicity of the compound towards Gram-positive bacteria.

b) antimicrobial effect on E. coli by placing a compound on a paper disk and determining the ability of the compound to inhibit the growth of the E. coli (Benson, Microbial Applications, 6^{th} Ed. Wm. C. Brown Publishers, Dubuque, Iowa (1994)). The results of this assay establish the toxicity of a compound towards Gram-negative bacteria.

c) antimicrobial effect on Candida albicans by placing a compound on a paper disk and determining the ability of the extract to inhibit the growth of Candida albicans (Benson, Microbial Applications, 6^{th} Ed. Wm. C. Brown Publishers, Dubuque, Iowa (1994)). The results of this study establish the toxicity of compounds towards yeasts and fungi.

d) multi drug resistance assay using S. aureus by placing a compound on a paper disk and determining the ability of the compound to inhibit the growth of S. aureus exhibiting methicillin resistance (clinical isolates provided by University of California, San Diego, Medical Center, #12144G) (Benson, Microbial Applications, 6^{th} Ed. Wm. C. Brown Publishers, Dubuque, Iowa (1994)). The results of this assay establish that bacteria that are resistant to methicillin are not resistant to the antibacterial effect of the compound.

e) multi-drug resistance assay using E. faecalis by placing a compound on a paper disk and determining the ability of the compound to inhibit the growth of E. faecalis exhibiting vancomycin resistance (clinical isolates provided by University of California, San Diego, Medical Center, #8673G) (Benson, Microbial Applications, 6^{th} Ed. Wm. C. Brown Publishers, Dubuque, Iowa (1994)). The results of this assay establish that bacteria that are resistant to vancomycin are not resistant to the antibacterial effect of the compound.

f) inhibition of the growth of cancer cells by contacting a compound with the National Cancer Institute's (NCI) cell line screen (approximately sixty cell lines) against up to fifty-one cancer cell types in vitro (Boyd et al., Drug Development Research, 34:91-109 (1995)). These results provide an activity profile for the compound. The activity profiles of an extract can be compared to the activity profiles of other samples in the NCI database of activity profiles. Similar activity profiles of different extracts, including known extracts with known modes of action, strongly suggests that the samples have similar modes of action. A novel activity profile strongly suggests that the compound has a novel mechanism of action.

g) immunomodulatory effects of a compound on the immune system in vitro or in vivo. For example, the modulation, increase or decrease of the activity of the cellular immune response, humoral immune response, or both, can be measured using methods known in the art. For example, T-cell response and B-cell response can be monitored by the type and amount of cytokines produced by a population of cells (such as T_{H}1 and T_{H}2 profiles), cytotoxic T-cell response can be determined using chromium release assays, B-cell response can be measured using immunoassays to detect the presence of specific antibodies, histamine release can be used to detect the presence and activity of mast cells, and the presence of absence of cell surface markers, such as CD4, CD3 or CD34 can be used to detect the presence or amount of cell populations in a sample. A wide variety of such tests are known in the art. (see, for example, Harrow, Antibodies: a Laboratory Manual, Cold Spring Harbor Press (1988); Roitt et al., Immunology, Third Edition, Mosby, St. Louis (1993); Zing et al., Biochem J. 319 (Pt. 1):159-165 (1996); Hess et al., J. Immunol. 141:3263-3269 (1998)). The results of these types of assays establish the immunomodulatory effects of a compound.

h) anti-inflammatory effects of a compound can be measured in vitro or in vivo. For example, animal models for such anti-inflammatory effects of compounds, such as the rabbit knee or mouse ear, can be used. In addition, the specificity of T-cell responses and B-cell responses in an inflammatory mode can be monitored by monitoring the type and response of such cells and their cytokine profiles (Bardley et al., J. Invest. Dermatology 78:206-209 (1982)). The results of these types of assays establish the anti-inflammatory activity of a compound.

i) radiation protective effects of a compound can be measured in vitro or in vivo. For example, cells in culture, a whole animal, including humans, or a portion of an animal can be exposed to a variety of doses of a compound before or after being exposed to a variety of doses and types of radiation, including ionizing radiation, preferably a dose and type of radiation used to treat cancers or tumors. The ability of the compound to protect the cells or animal can be measured using methods known in the art. For example, the ability of the cells or animal to survive longer, or the ability of the cells or animal to be in a healthier state, when treated with an extract indicates that the compound has a radiation protective effect (Grant et al., Blood 83:663-667 (1994)). The results of these types of assays establish the radiation protective activity of an compound.

j) PKC modulating effects of a compound can be measured by contacting a compound with a sample including at least a portion of a PKC, including a cell, and determining the modulation of PKC activity or the binding of the compound to PKC. Such methods are known in the art (see, DeVries 1988).

k) cytotoxic activity of a compound can be determined by a variety of methods, including inhibition of brine shrimp by contacting twenty-four hour old brine shrimp nauplii for twenty-four hours with an compound and observing the inhibition of the activity or viability of the brine shrimp. The results of this assay establish the cytotoxicity of the compound towards whole organisms.

Compounds identified as having a bioactivity have presumptive therapeutic activity. Such therapeutic activity and related pharmacological parameters can be confirmed using the methods discussed herein.

Pharmacology and Toxicity of Bioactive Compounds and Bioactivities

The structure of a bioactive compound or bioactivity can be determined or confirmed by methods known in the art, such as mass spectroscopy. For bioactive compounds and bioactivities stored for extended periods of time under a variety of conditions, the structure, activity and potency thereof can be confirmed.

Identified bioactive compounds and bioactivities can be evaluated for a particular activity using are-recognized methods and those disclosed herein. For example, if an identified bioactive compound or bioactivity is found to have anticancer cell activity in vitro, then the bioactive compound or bioactivity would have presumptive pharmacological properties as a chemotherapeutic to treat cancer. Such nexuses are known in the art for several disease states, and more are expected to be discovered over time. Based on such nexuses, appropriate confirmatory in vitro and in vivo models of pharmacological activity, and toxicology, and be selected and performed. The methods described herein can also be used to assess pharmacological selectivity and specificity, and toxicity.

Identified bioactive compounds and bioactivities can be evaluated for toxicological effects using known methods (see, Lu, Basic Toxicology, Fundamentals, Target Organs, and Risk Assessment, Hemisphere Publishing Corp., Washington (1985); U.S. Pat. Nos; 5,196,313 to Culbreth (issued Mar. 23, 1993) and 5,567,952 to Benet (issued Oct. 22, 1996)). For example, toxicology of a bioactive compound or bioactivity can be established by determining in vitro toxicity towards a cell line, such as a mammalian, for example human, cell line. Bioactive compounds and bioactivities can be treated with, for example, tissue extracts, such as preparations of liver, such as microsomal preparations, to determine increased or decreased toxicological properties of the bioactive compound of bioactivity after being metabolized by a whole organism. The results of these types of studies are predictive of toxicological properties of chemical s in animals, such as mammals, including humans.

Alternatively, or in addition to these in vitro studies, the toxicological properties of a bioactive compound or bioactivity in an animal model, such as mice, rats, rabbits, dogs or monkeys, can be determined using established methods (see, Lu, supra (1985); and Creasey, Drug Disposition in Humans, The Basis of Clinical Pharmacology, Oxford University Press, Oxford (1979)). Depending on the toxicity, target organ, tissue, locus and presumptive mechanism of the bioactive compound or bioactivity, the skilled artisan would not be burdened to determine appropriate doses, LD₅₀ values, routes of administration and regimes that would be appropriate to determine the toxicological properties of the bioactive compound or bioactivity. In addition to animal models, human clinical trials can be performed following established procedures, such as those set forth by the United States Food and Drug Administration (USFDA) or equivalents of other governments. These toxicity studies provide the basis for determining the efficacy of a bioactive compound or bioactivity in vivo.

Efficacy of Bioactive Compounds and Bioactivities

Efficacy of a bioactive compound or bioactivity can be established using several art recognized methods, such as in vitro methods, animal models or human clinical trials (see, Creasey, supra (1979)). Recognized in vitro models exist for several diseases or conditions. For example, the ability of a compound or composition to extend the life-span of HIV-infected cells in vitro is recognized as an acceptable model to identify chemicals expected to be efficacious to treat HIV infection or AIDS (see, Daluge et al., Antimicro. Agents Chemother. 41:1082-1093 (1995)). Furthermore, the ability of cyclosporin A (CsA) to prevent proliferation of T-cells in vitro has been established as an acceptable model to identify chemicals expected to be efficacious as immunosuppressants (see, Suthanthiran et al., supra (1996)). For nearly every class of therapeutic, disease or condition, an acceptable in vitro or animal model is available. In addition, these in vitro methods can use tissue extracts, such as preparations of liver, such as microsomal preparations, to provide a reliable indication of the effects of metabolism on a bioactive compound or bioactivity. Similarly, acceptable animal models can be used to establish efficacy of bioactive compounds and bioactivities to treat various diseases or conditions. For example, the rabbit knee is an accepted model for testing agents for efficacy in treating arthritis (see, Shaw and Lacy, J. Bone Joint Surg. (Br.) 55:197-205 (1973)). Hydrocortisone, which is approved for use in humans to treat arthritis, is efficacious in this model which confirms the validity of this model (see, McDonough, Phys. Ther. 62:835-839 (1982)). When choosing an appropriate model to determine efficacy of bioactive compounds and bioactivities, the skilled artisan can be guided by the state of the art to choose an appropriate model, doses and route of administration, regime and endpoint and as such would not be unduly burdened.

In addition to animal models, human clinical trials can be used to determine the efficacy of bioactive compounds and bioactivities. The USFDA, or equivalent governmental agencies, have established procedures for such studies.

Selectivity of Bioactive Compounds and Bioactivities

The in vitro and in vivo methods described above also establish the selectivity of a candidate modulator. It is recognized that chemicals can modulate a wide variety of biological processes or be selective. Panels of cells as they are known in the art can be used to determine the specificity of the a bioactive compound or bioactivity (WO 98/13353 to Whitney et al., published Apr. 2, 1998). Selectivity is evident, for example, in the field of chemotherapy, where the selectivity of a chemical to be toxic towards cancerous cells, but not towards non-cancerous cells, is obviously desirable. Selective modulators are preferable because they have fewer side effects in the clinical setting. The selectivity of a bioactive compound or bioactivity can be established in vitro by testing the toxicity and effect of a bioactive compound or bioactivity can be established in vitro by testing the toxicity and effect of a bioactive compound or bioactivity on a plurality of cell lines that exhibit a variety of cellular pathways and sensitivities. The data obtained form these in vitro toxicity studies can be extended to animal model studies, including human clinical trials, to determine toxicity, efficacy and selectivity of a bioactive compound or bioactivity.

The selectivity, specificity and toxicology, as well as the general pharmacology, of a bioactive compound or bioactivity can be often improved by generating additional test chemicals based on the structure/property relationship of a bioactive compound or bioactivity originally identified as having activity. Bioactive compounds and bioactivities can be modified to improve various properties, such as affinity, life-time in blood, toxicology, specificity and membrane permeability. Such refined bioactive compounds and bioactivities can be subjected to additional assays as they are known in the art or described herein. Methods for generating and analyzing such compounds or compositions are known in the art, such as U.S. Pat. No. 5,574,656 to Agrafiotis et al.

Pharmaceutical Compositions

The present invention also encompasses a bioactive compound or bioactivity in a pharmaceutical composition comprising a pharmaceutically acceptable carrier prepared for storage and preferably subsequent administration, which have a pharmaceutically effective amount of the bioactive compound or bioactivity in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The bioactive compounds and bioactivities of the present invention can be formulated and used as tablets, capsules or elixirs for oral administration; suppositories for rectal administration,; sterile solutions, suspensions or injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparation, such as liposomes, can be used.

The pharmaceutically effective amount of a bioactive compound or bioactivity required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present invention, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including parenterally, intravenously, subcutaneously, intramuscularly, colonically, rectally, nasally or intraperiotoneally, employing a variety of dosage forms. Such methods can also be used in testing the activity of bioactive compounds or bioactivities in vivo.

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above. Typically, human clinical applications of products are commenced at lower dosage levels, with dosage level being increased until the desired effect is achieved. Alternatively, acceptable in vitro studies can be used to establish useful doses and routes of administration of the bioactive compounds and bioactivities.

In non-human animal studies, applications of the pharmaceutical compositions are commenced at higher dose levels, with the dosage being decreased until the desired effect is no longer achieved or adverse side effects are reduced or disappear. The dosage for the bioactive compounds and bioactivities of the present invention can range broadly depending upon the desired affects, the therapeutic indication, route of administration and purity and activity of the bioactive compound or bioactivity. Typically, dosages can be between about 1 ng/kg and about 10 ng/kg, preferably between about 10 ng/kg and about 1 mg/kg, more preferably between about 100 ng/kg and about 100 micrograms/kg, and most preferably between about 1 microgram/kg and about 10 micrograms/kg.

The exact formulation, route of administration and dosage can be chosen by the individual physician in view of the patient's condition (see, Fingle et al., in The Pharmacological Basis of Therapeutics (1975)). It should be noted that the attending physician would know how to and when to terminate, interrupt or adjust administration due to toxicity, organ dysfunction or other adverse effects. Conversely, the attending physician would also know to adjust treatment to higher levels if the clinical response were not adequate. The magnitude of an administrated does in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight and response of the individual patient, including those for veterinary applications.

Depending on the specific conditions being treated, such pharmaceutical compositions can be formulated and administered systemically or locally. Techniques for formation and administration can be found in Remington's Pharmaceutical Sciences, 18th Ed., Mack Publishing Co., Easton, Pa. (1990). Suitable routes of administration can include oral, rectal, transdermal, otic, ocular, vaginal, transmucosal or intestinal administration; parenteral delivery, including intramuscular, subcutaneous, intramedullary injections, as well as intrathecal, direct intraventricular, intravenous, intraperitoneal, intranasal, or intraocular injections.

For injection, the pharmaceutical compositions of the present invention can be formulated in aqueous solutions, preferably in physiologically compatible buffers such as Hanks' solution, Ringer's solution or physiological saline buffer. For such transmucosal administration, penetrans appropriate to the barrier to be permeated are used in the formulation. Such penetrans are generally known in the art. Use of pharmaceutically acceptable carriers to formulate the pharmaceutical compositions herein disclosed for the practice of the invention into dosages suitable for systemic administration is within the scope of the invention. With proper choice of carrier and suitable manufacturing practice, the compositions of the present invention, in particular, those formulation as solutions, can be administered parenterally, such as by intravenous injection. The pharmaceutical compositions can be formulated readily using pharmaceutically acceptable carriers well known in the art into dosages suitable for oral administrations. Such carriers enable the bioactive compounds and bioactivities of the invention to be formulated as tables, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Substantially all molecules present in an aqueous solution at the time of liposome formation are incorporated into or within the liposomes thus formed. The liposomal contents are both protected from the external micro-environment and, because liposomes fuse will cell membranes, are efficiently delivered into the cell cytoplasm. Additionally, due to their hydrophobicity, small organic molecules can be directly administered intracellularly.

Pharmaceutical compositions suitable for use in the present invention include compositions wherein the active ingredients are contained in an effective amount to achieve its intended purpose. Determination of the effective amount of a pharmaceutical composition is well within the capability of those skilled in the art, especially in light of the detailed disclosure provided herein. In addition to the active ingredients, these pharmaceutical compositions can contain suitable pharmaceutically acceptable carriers comprising excipients and auxiliaries which facilitate processing of the active chemicals into preparations which can be used pharmaceutically. The preparations formulated for oral administration may be in the form of tables, dragees, capsules or solutions. The pharmaceutical compositions of the present invention can be manufactured in a manner that is itself known, for example by means of conventional mixing, dissolving, granulating, dragee-making, emulsifying, encapsulating, entrapping or lyophilizing processes. Pharmaceutical formulations for parenteral administration include aqueous solutions of active chemicals in water-soluble form.

Additionally, suspensions of the active chemicals may be prepared as appropriate oily injection suspensions. Suitable lipophilic solvents or vehicles include fatty oils such as sesame oil, or synthetic fatty acid esters, such as ethyl oleate or triglycerides or liposomes. Aqueous injection suspensions may contain substances what increase the viscosity of the suspension, such as sodium carboxymethyl cellulose, sorbitol or dextran. Optionally, the suspension can also contain suitable stabilizers or agents that increase the solubility of the chemicals to allow for the preparation of highly concentrated solutions.

Pharmaceutical compositions for oral use can be obtained by combining the active chemicals with solid excipient, optionally grinding a resulting mixture, and processing the mixture of granules, after adding suitable auxiliaries, if desired, to obtain tables or dragee cores. Suitable excipients are, in particular, fillers such as sugars, including lactose, sucrose, mannitol or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose and/or polyvinylpyrrolidone. If desired, disintegrating agents can be added, such as the cross-linked polyvinyl pyrolidone, agar, alginic acid or a salt thereof such as sodium alginate. Dragee cores can be provided with suitable coatings. Dyes or pigments can be added to the tablets or dragee coatings for identification or to characterize different combinations of active doses.

The bioactive compounds and bioactivities of the present invention, and pharmaceutical compositions that include such bioactive compounds and bioactivities are useful for treating a variety of ailments in a patient, including a human. As set forth in the Examples, the bioactive compounds and bioactivities of the present invention have antibacterial, antimicrobial, antiviral, anticancer cell, antitumor and cytotoxic activity. A patient in need of such treatment can be provided a bioactive compound or bioactivity of the present invention, preferably in a pharmacological composition in an effective amount to reduce the number or growth rate of bacteria, microbes, cancer cells or tumor cells in said patent, or to reduce the infectivity of viruses in said patient. The amount, dosage, route of administration, regime and endpoint can all be determined using the procedures described herein.

VI. Bacterial Symbionts of Bugula

The present invention includes a preparation of at least one bacterial symbiont of B. neritina or B. pacifica, wherein said bacterial symbiont comprises at least one polypeptide that has at least one PKS activity. The preparation of at least one bacterial symbiont can be substantially free of its host Bugula and can be provided as an isolated preparation or isolated culture (only one type of bacteria) or a mixed preparation or mixed culture (more than one type of bacteria. As set forth in the Examples, bacterial symbionts of Bugula neritina and Bugula pacifica are involved in the production of polyketides included bryopyran rings and bryostatins.

VII. Polyketides, Bryopyrans and Bryostatins from Bugula pacifica and Symbionts

The present invention also includes at least one bioactive compound present in a Bugula pacifica. The bioactive compound is preferably a polyketide, a bryopyran ring or a bryostatin. The composition preferably has at least one activity of at least one bryostatin, which can be confirmed using methods of the present invention. The composition can be made by isolating the composition from B. pacifica using methods of the present invention, such as by extraction from B. pacifica using appropriate solvents, such as ethanol. The extracts can be separated to obtain pure or substantially pure compounds using methods such as HPLC. The bioactivity of these compounds, either alone or in combination, can be confirmed using methods of the present invention. Such compounds can be provided in a pharmaceutically acceptable carrier and can be provided as a pharmaceutical composition.

### EXAMPLES

### Example 1

### Extraction of Nucleic Acid Molecules From Samples of B. neritina That Include Symbionts

Unless otherwise noted, molecular biology procedures used are standard techniques used in the field (Sambrook et al., 1989).

DNA Extraction

Samples of either adult or larval B. neritina were obtained from the waters of southern California. Free-swimming larvae were collected in the lab from adult B. neritina colonies. Larval DNA was extracted as previously described (Haygood and Davidson, 1997). Larvae were concentrated in 1.5 milliliter microcentrifuge tubes (approximately 25 mg of larvae per tube) by gentle centrifugation and then rinsed four times in filtered seawater (0.2 micrometer pore size filter) to minimize the contaminating seawater bacteria. Excess water was removed and the pellets were frozen at −80° C. for later use. DNA was extracted from the B. neritina larvae using a QIAamp Tissue Kit as directed by the manufacturer (Qiagen Inc., Valencia, Calif.).

Adult B. neritina total DNA was extracted using a modification of the method of Shure et al. (Shure et al., 1983). Briefly, two grams of fresh adult B. neritina was frozen on dry ice and pulverized in a mortar. The powdered tissue was taken up in 6 ml of extraction buffer containing 8 M urea, 0.35 M NaCl, 0.05 M Tris-HCl (pH 7.5), 0.02 M EDTA and 2% sarcosyl. An equal volume of phenol/chloroform/isoamyl alcohol (25:24:1), pH 8.0 was added and mixed thoroughly. After 15 minutes at room temperature, the solution was again thoroughly mixed and centrifuged at 8,000 rpm for 10 minutes at 4° C. to separate the phases. The supernatant was transferred to a clean tube and a second phenol/chloroform/isoamyl alcohol extraction was performed. The supernatant was transferred to a clean tube and the salt concentration was increased by the addition of 1/10 volume 3 M sodium acetate, pH 5.2 and a volume of isopropanol equal to the new total volume. The solution was mixed well and centrifuged at 8,000 rpm for 10 min to collect the pellet. The pellet was washed with 70% ethanol and air dried for 10 min. The DNA was dissolved in approximately ⅕ the original lysis volume of 10 mM Tris-HCl (pH 7.5), 10 mM EDTA. The DNA was subsequently run through a Sephadex G-200 spin column (Maloy et al., 1996) to remove any PCR inhibitors.

### Example 2

### Identification of PKS Genes in Samples of B. neritina That Include Symbionts

Primer Design

Degenerate PCR primers (PKSR and BLCASPKS) were designed based on conserved regions of the beta-ketoacyl synthase (KS) domain of PKS-I genes.

TABLE I
PCR primers designed for this study.

| Primer | | |
|---|---|---|
| Designation | | SEQ |
| and | | ID |
| Orientation | Sequence | NO. |
| PKSR | ACR TGI GCR TTI GTI CC | SEQ |
| (forward) | | ID |
| | | NO: 1 |
| BLCASPKS | ICA YGG IAC IGG IAC | SEQ |
| (reverse) | | ID |
| | | NO: 2 |
| SWA38R | ACG GAC AAG CGT CAT TAC | SEQ |
| (forward) | | ID |
| | | NO: 3 |
| SWA38L | GCC AAG GCT TTA ATT CCG | SEQ |
| (reverse) | | ID |
| | | NO: 4 |
| SWA38F3 | GTT GTC TTT GCA GCA TCG CAT GTT ACC | SEQ |
| (forward) | AC | ID |
| | | NO: 5 |
| SWA38R3 | CAC GCC CGC TAT CCC AGC ACC TAC C | SEQ |
| (reverse) | | ID |
| | | NO: 6 |

PCR conditions for amplification of DNA from B. neritina

The PCR conditions for the initial amplification of the KS genes from B. neritina DNA were as follows. A total reaction volume of 50 microliter contained approximately 100 ng of B. neritina DNA (either adult or larval), 1 micromolar each primer (PKSR and BLCASPKS), and Taq polymerase and buffer (Boehringer Mannheim Corp., Indianapolis, Ind.). A PCR protocol was optimized for the degenerate KS primers PKSR and BLCASPKS. The cycle conditions started with a “touch down” sequence, which lowered the annealing temperature from 60 to 40° C. at a rate of 2° C. per cycle (11 cycles), and were then maintained at 40° C. for a total of 51 cycles. Cycle steps were as follows: denaturation (94° C.; 1 min), annealing (60° C. to 40° C.; 2 min), and extension (72° C.; 1 min). PCR conditions for the KSa specific primers (SWA38R and SWA38L) were as follows: denaturation (94° C.; 1 min), annealing (60° C.; 1 min), and extension (72° C.; 2 min).

Inverse PCR

To obtain flanking DNA sequence, the most prevalent clone, KSa, was extended using inverse PCR (Ochman et al., 1990). Adult B. neritina DNA was digested with the restriction enzyme Sau3A1 and then religated using T4 DNA ligase (Stratagene, La Jolla, Calif.). KSa specific primers (SWA 38F3 and SWA 38R3) flanking the Sau3A1 restriction site, were used to PCR amplify from the ligation reaction using TaqPlusLong polymerase (Stratagene, La Jolla, Calif.) as recommended by the manufacturer using the PCR conditons listed above for primers SWA38R and SWA38L.

DNA Cloning

PCR reactions were electrophoresed on 0.8% agarose gel and visualized with ethidium bromide. PCR products of approximately 300 bp were cloned using a TOPO TA Cloning kit into Invitrogen pCR® 2.1-TOPO vector as described by the manufacturer (Invitrogen Corp., Carlsbad, Calif.). Recombinant clones containing insert DNA were then sequenced using standard protocols. Since PKS-I enzymes are modular, clones from the degenerate PCR primers represents a pool of fragments from different KS domains.

DNA Sequencing

Plasmid DNA for sequencing was prepared using the Qiagen QIAprep Spin Miniprep Kit (Qiagen, Inc., Valencia, Calif.). All sequencing was performed with an ABI automated sequencer (model 373A) by using a PRISM Ready Reaction DyeDeoxy terminator cycle sequencing kit as recommended by the manufacturer (Perkin-Elmer). Cloned genes were sequenced using primers directed against the cloning vector, pCR® 2.1-TOPO (Invitrogen, Carlsbad, Calif.).

### Example 3

### Sequences of PKS from B. neritina That Include Symbionts

Degenerate primers (PKSR and BLCASPKS) were designed to conserved regions of the KS domains of the bacterial PKS-I. These were used in a step down PCR to amplify a 300 bp fragment from B. neritina DNA. Since PKS-I enzymes are modular, these PCR products are a pool of fragments from different PKS modules, and were cloned before sequencing. Two clone libraries were prepared, one from adult B. neritina DNA and one from larval DNA. Twenty-seven clones have been sequenced (Table II).

TABLE II
cDNA Clones obtained from B. neritina

| Clone Designation | Number of Isolates | Source (Larval or Adult) |
|---|---|---|
| KSa | 13 | Larval |
| KSb | 3 | Larval and Adult |
| KSc | 2 | Adult |
| KSd | 2 | Adult |
| KSe | 3 | Larval |
| KSf | 1 | Adult |
| KSg | 1 | Adult |
| KSh | 1 | Larval |
| KSi | 1 | Adult |

Nine unique clones have been identified (KSa-KSi) (Table III). One clone (KSb) appeared in both libraries. Cloned DNA sequences were identified by using the BLAST (basic local alignment search tool) server of the National Center for Biotechnology Information accessed over the Internet (Altschul et al., 1997). All of these sequences have signature regions for KS and show highest similarity in BLAST searches to bacterial PKS-I showing that they are in fact of PKS-I origin.

TABLE III
Sequences of (KSa-KSi) clones obtained from B. neritina and
predicted amino acid products

| SEQ ID NO. for clones | SEQ ID NO. for amino acids |
|---|---|
| SEQ ID NO: 9 | SEQ ID NO: 10 |
| SEQ ID NO: 13 | SEQ ID NO: 14 |
| SEQ ID NO: 15 | SEQ ID NO: 16 |
| SEQ ID NO: 17 | SEQ ID NO: 18 |
| SEQ ID NO: 19 | SEQ ID NO: 20 |
| SEQ ID NO: 21 | SEQ ID NO: 22 |
| SEQ ID NO: 23 | SEQ ID NO: 24 |
| SEQ ID NO: 25 | SEQ ID NO: 26 |
| SEQ ID NO: 27 | SEQ ID NO: 28 |

Inverse PCR from the most prevalent clone, KSa, yielded additional sequence for a total of 737 bp of contiguous DNA (SEQ ID NO:13). (The predicted amino acid product is presented in SEQ ID NO:14.) This sequence is similar to the 3′ end of the KS domain and an ‘intermodular’ region upstream of the acyl transferase (AT) domain when aligned with the pikAI KS domain from Streptomyces venezuelae (Xue et al., 1998) and DEBSI from Saccharopolyspora erythraea (Donadio et al., 1991). This sequence represents a single ORF as predicted for a PKS-I. In total, 3.2 kb of DNA has been cloned from adult and larval B. neritina DNA. All of the clones exhibit significant similarity to other PKS-I genes as illustrated by BLAST searches (Altschul et al., 1997). The GC content of the B. neritina clones is very low, ranging from about 32% to about 53%. This is lower than any other known bacterial PKS-I. This underscores the novelty of these genes. Due to the low GC content, it is unlikely that these PKS genes were obtained by lateral transfer from actinomycetes as suggested for other PKS genes, such as those from the myxobacterium Sorangium cellulosum (Schupp et al., 1995).

As SSU rRNA sequencing has shown, E. sertula is a γ-proteobacterium closely related to Pseudomonas fluorescens and P. syringae (Haygood and Davidson, 1997). However, the GC content of the Pseudomonas PKS genes is much higher, 63% for Pseudomonas fluorescens (Nowak-Thompson et al., 1997) and 67% for P. syringae (Rangaswamy et al., 1998). Thus, the PKS genes isolated here from B. neritina DNA (and presumably E. sertula) are unique, even among other closely related γ-proteobacterium. A genetic feature shared by the myxobacteria and actinomycetes is a high GC % content (67 to 71 and 69-73 mol %, respectively) (Seow et al., 1997; Schupp et al., 1995). Again, this reinforces the novel nature of the B. neritina/E. sertula derived PKS genes.

When the degenerate oligonucleotides were used in step down PCR with B. neritina DNA as a template, PCR products of approximately 300 bp were obtained using adult DNA from several locations and seasons. Single PCR products of approximately 300 bp were evident in amplifications from either adult or larval B. neritina DNA (containing both host bryozoan DNA and microbial symbiont DNA, presumably E. sertula). The consistent amplification of this product from both adult and larval B. neritina DNA suggests that it is not due to a sporadic contaminant. These PCR products are of the expected size based upon the location of the primers within the KS domains of other PKS-I genes.

Link Between the KSa Gene, B. neritina, and Bryostatins

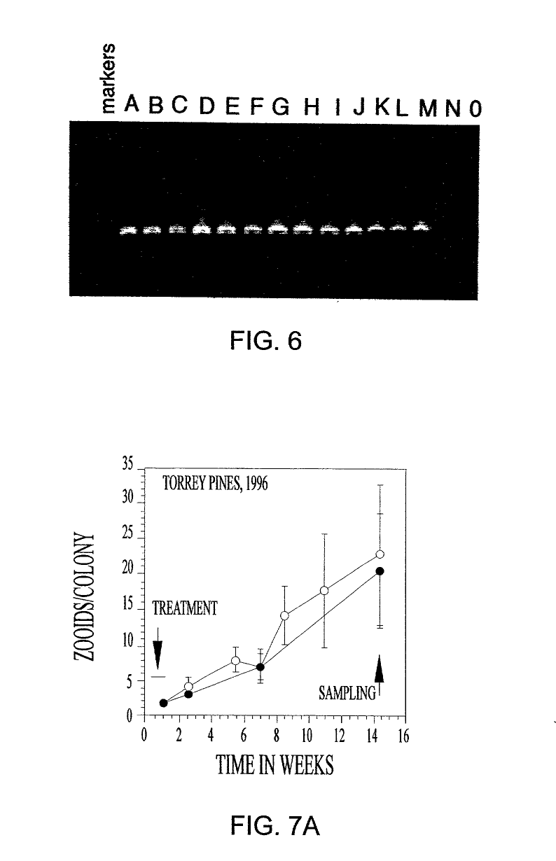

Specific primers (SWA38R and SWA38L) were designed against variable regions from an abundant clone, KSa, isolated from the B. neritina larval library. These primers were designed to PCR amplify only this fragment, unlike the degenerate primers used to obtain the libraries, which amplify from any KS domain. Ten samples of DNA from adult colonies from a wide range of locations along the California coast from San Diego to Humboldt Bay, one adult sample from North Carolina, and two larval samples were screened for a KSa specific sequence by PCR amplification. All produced a strong band with the KSa specific primers (FIG. 6), lanes A-M). Two samples of other bryozoans collected together with B. neritina were negative (FIG. 6), lanes N, O). This result demonstrates that KSa is universal in B. neritina. These characteristics are those expected for a fragment derived from bryopyran synthase. KSb, which was independently isolated from adult and larval amplifications is another strong candidate as a sequence originating in bryopyran synthase. Based on the structure of other PKS genes the present inventors expect twelve KS domains in bryopyran synthase are expected.

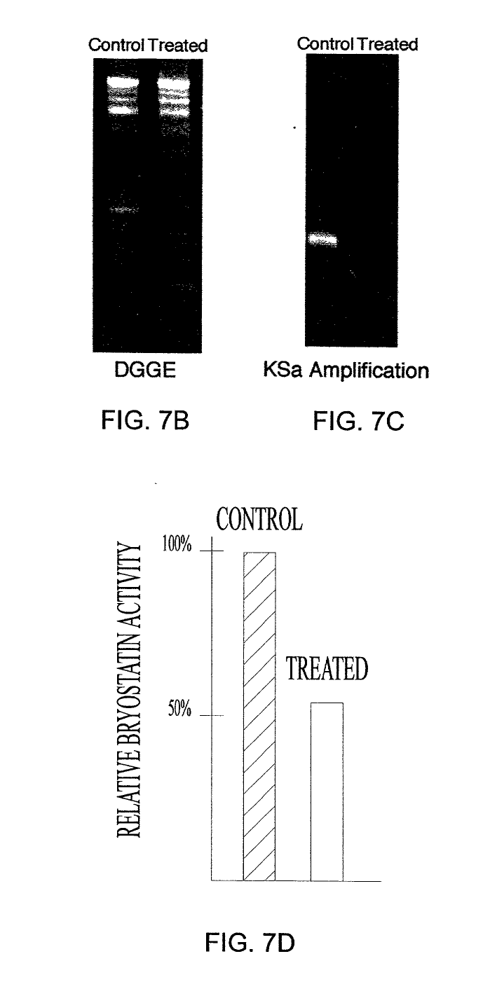

B. neritina larvae were treated with gentamicin at about 100 micrograms per milliliter for about seven days post-settlement, grown out for 3 months to re-establish their commensal bacterial flora, and then assayed for E. sertula levels and KSa levels by specific PCR and bryostatin activity by phorbol dibutyrate displacement (PdBu) assay (FIG. 7). The standard method for detecting bryostatins is the PdBu displacement assay (DeVries et al., 1988). Rat brain liposomes are incubated with tritiated PdBu, which binds to PKC in the liposomes. The liposomes are collected by filtration and counted in a scintillation counter. If an extract is added that contains a compound that can compete with phorbol for binding, less tritiated PdBu is bound. The assay has a subnanomolar detection limit for pure bryostatin 1 and is suitable for milligram quantities of sample. In B. neritina, the only compounds with PdBu activity are bryostatins, and PdBu activity is a good measure of total bryostatin activity.

Both E. sertula population and KSa levels were reduced dramatically and similarly and bryostatin activity was also reduced compared to untreated controls (FIG. 7). Denaturing gradient gel electrophoresis (DGGE) (Muyzer et al., 1993) experiments showed that E. sertula is the only bacterium whose population is reduced in treated colonies, and growth data showed the bryozoan growth rates were the same in treated and untreated colonies. The concomitant reduction of KSa suggests that this gene resides in E. sertula and is involved in bryostatin synthesis.

### Example 4

### Cloning of a PKS Gene Cluster from Endobugula sertula

Preparation of High Molecular Weight DNA from Burgula neritina for Cosmid Cloning

Tips from fresh Bugula neritina contain the symbiont E. sertula and were excised with scissors, blotted dry, and frozen at −80° C. in 8 gram aliquots. DNA was extracted by pulverizing small portions of one aliquot with a mortar and pestle chilled with dry ice, to keep the tissue frozen. After all of the tissue was pulverized it was added to 24 milliliters of lysis buffer (50 mM Tris, pH 8.0, 50 mM Na₂EDTA, 350 mM NaCl, 2% Na sarcosyl, 8 M urea) in a 50 milliliters screw cap tube. The sample was incubated for 5 minutes at room temperature, and then 10 milliliters of phenol:chloroform (1:1) was added. The tube was placed on a rotator and the aqueous and organic layers mixed for 40 minutes at 20 rpm. The tube was then centrifuged in a table top centrifuge for 5 minutes at maximum speed, and the upper layer gently transferred with a wide-bore pipet to a new tube. Ten milliliters of phenol:chloroform was added and the sample rotated for 25 minutes at 20 rpm. After centrifugation, the upper layer was transferred to a new tube, 10 milliliters of phenol:chloroform added, and the tube rotated for 20 minutes at 20 rpm. After centrifugation, the upper layer was removed, divided in half, and each half ethanol precipitated by adding one-tenth volume of 3 molar Na acetate, and 2 volumes of ethanol. The tubes were then centrifuged in a Sorval RC5B centrifuge for 10 minutes at 10,000 rpm in an HB4 rotor, and the pellets were washed twice with 70% ethanol. One-half milliliter of TE buffer (10 mM Tris-HCl, pH 8.0, 1 mM Na₂EDTA) was added to each tube and the tubes were incubated with gentle shaking at 18° C. overnight. Pipeting was done with a cut-off pipetman tip. DNA was stored at 4° C.

Partial Digestion and Sucrose Gradient Fractionation of B. neritina DNA

B. neritina DNA was then partially digested with Sau3AI for cloning by the following protocol. Pilot experiments showed that between 0.04-0.08 units of enzyme per microliter were required in a reaction containing 0.1 microgram per microliter DNA, incubated for 1 hour at 37° C. Large scale digests were set up using 100 micrograms of DNA and 0.04, 0.06, and 0.08 units of enzyme per microliter, in 1 milliliter total volume, incubated at 37° C. for 1 hour. Forty microliters of 500 millimolar Na₂EDTA was added to each tube, and samples heated to 70° C. for 15 min. After cooling, aliquots were run on a 0.5% agarose gel to check the extent of digestion. All three treatments gave fragments within the desired size range of 30 to 50 kbp. Samples were phenol:chloroform extracted twice, ethanol precipitated, washed twice in 70% ethanol and resuspended in 200 microliters TE buffer (10 mM Tris-HCl, 1 mM Na₂EDTA). Samples were loaded on 10-40% sucrose gradients (made in 10 mM Tris-HCl, pH 8.0, 10 mM NaCl, 1 mM Na₂EDTA), and centrifuged in a SW41 rotor in a Beckman LC8 centrifuge for 22 hours at 22,000 rpm at 20° C. After completion, gradients were fractionated by removing 350 microliter aliquots from the top with a pipetman. Fractions were analyzed by running aliquots on an agarose gel. Appropriate fractions were precipitated by adding 150 microliters TE, 6 micrograms tRNA as carrier, 50 microliters 3 molar Na acetate, and 1 milliliter ethanol. Tubes were placed on ice 45 minutes, centrifuged 30 minutes at 10,000 rpm in an HB4 rotor, washed twice with 70% ethanol, and resuspended in 20 microliters water.

Competitive PCR of B. neritina DNA

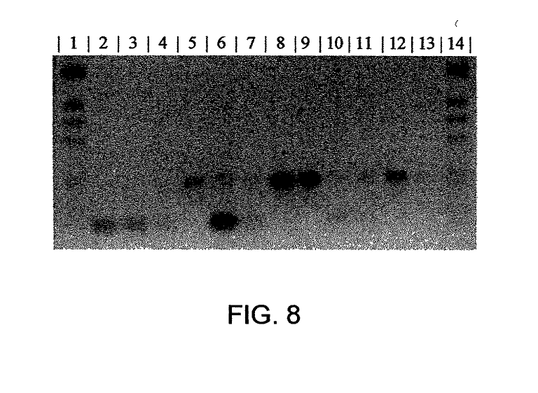

To determine whether the fractionated, partially-digested DNA preparation from B. neritina, had enough representation of E. sertula DNA for cloning, competitive PCR was performed, using a clone containing a deletion in KSa. An unfractionated preparation of DNA previously obtained by an alternate method was used as a control. Fraction number 18 from the 0.06 unit/microliter Sau3AI digest was used as representative of the B. neritina DNA preparation of the more gently isolated DNA of this example. The results are shown in FIG. 8. Bacterial DNA in the fractionated DNA preparation amplified with equal intensity to the competitor at a 10-fold higher level of competitor and is indicative of bacterial DNA present at 10-fold higher levels in the DNA preparation of this example compared with the previous isolated preparation. Based on estimations of bacterial and host genome size, there were about 6 bacterial genome equivalents per host genome equivalent. From this information it was determined that 14,000 cosmid clones were required to ensure a 95% probability of representation of a given gene.

Ligation and Packaging of DNA

Three sucrose gradient fractions (no. 19, 21, and 23) from the 0.06 unit/microliter Sau3AI digest were used for ligations consisting of 18 microliter insert DNA (approx. 2 micrograms), 1 microliter (1 microgram) of SuperCos vector (Statagene, Inc., La Jolla, Calif.), that had been digested with BamHI and phosphatased, and 5 units of T4 DNA ligase. After overnight incubation at room temperature, a portion was removed to check the efficiency of ligation on an agarose gel, and the remainder was ethanol precipitated, washed in 70% ethanol and resuspended in 20 microliters TE. Two microliters were used for packaging using the GigaPack Gold III kit (Stratagene, Inc., La Jolla, Calif.). In a screen of 12 colonies it was determined that 83% of clones had cosmid-sized inserts. Packaging was repeated with four more aliquots, with a final estimate of 40,000 colony forming units total. After plating all of the packaging mixes about 10,000 colonies were present and screened for the presence of KSa.

Colony Screening and Selection of Positive Clones

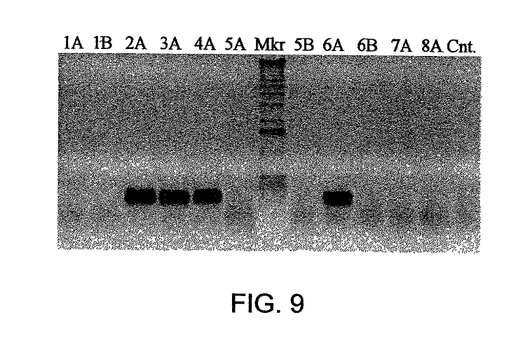

Colony filters were screened by standard procedures, using a ³²P-labeled probe derived from the cloned KSa fragment. Eleven colonies appeared positive and were picked onto another plate. These were screened by PCR using the KSa-specific primers, and four clones designated 2A, 3A, 4A, and 6A, gave positive amplification (FIG. 9). These were grown for midi-preparation of cosmid DNA.

DNA Preparation and Analysis of KSa-hybridizing Cosmid Clones

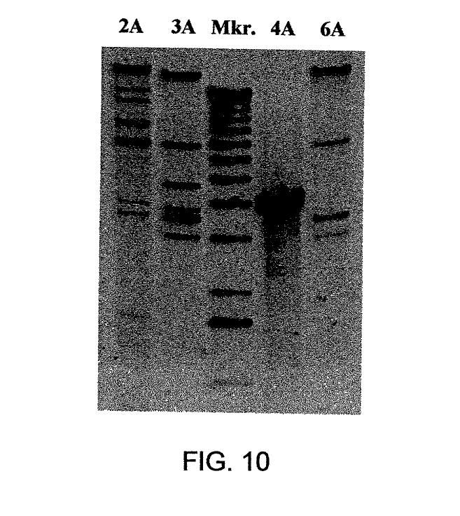

DNA preparations for cosmid clones were done by growing 100 milliliters culture in Luria Broth medium, and using a Midi-Prep kit (Qiagen, Inc., Valencia, Calif.) for isolation. EcoRI restriction digests of the four clones showed that 2A, 3A, and 6A appeared to have common fragments (FIG. 10), suggesting that the clones overlapped. Clone 4A had apparently lost its insert and repeated attempts to isolate this clone were unsuccessful. Clone 2A contained fragments with different stoichiometries, suggesting that two colonies were picked in the original isolation or the clone was deleting a segment. Further attempts to isolate a full length copy of 2A were unsuccessful. Clones 3A and 6A had high yields of DNA and appeared normal and were further characterized.

Sequencing and Restriction Mapping of Clones 3A and 6A

All sequencing reactions on cosmid clones were done using a Dye Terminator Cycle Sequencing Kit with AmpliTaq® DNA Polymerase FS (Applied Biosystems Inc., Foster City, Calif.), and reactions run on an ABI 373A sequencer. Initial sequencing reactions on clones 3A and 6A were done using T7 and T3 primers, and the KSa-specific primers. Results confirmed that the KSa region was present in these clones, and that the T3 ends of 3A and 6A had homology to PKS genes. Restriction maps were generated by identifying common fragments in overlapping regions of clones, in combination with hybridization analysis with the KSa probe, and oligonucleotide probes hybridizing to flanking sequences in the SuperCos vector. After thorough mapping and sequencing of the clones, it was determined that PKS homology was located only near the T3 ends of both clones. Since a PKS cluster with the capability to synthesize bryostatin could be as large as 60 kbp, overlapping clones extending beyond the T3 end of 6A were required.

Isolation of Cosmid Clones 5A and 5B

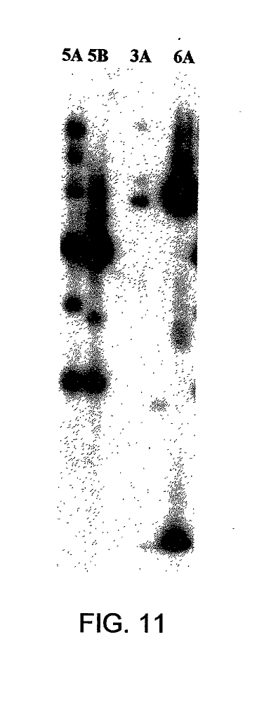

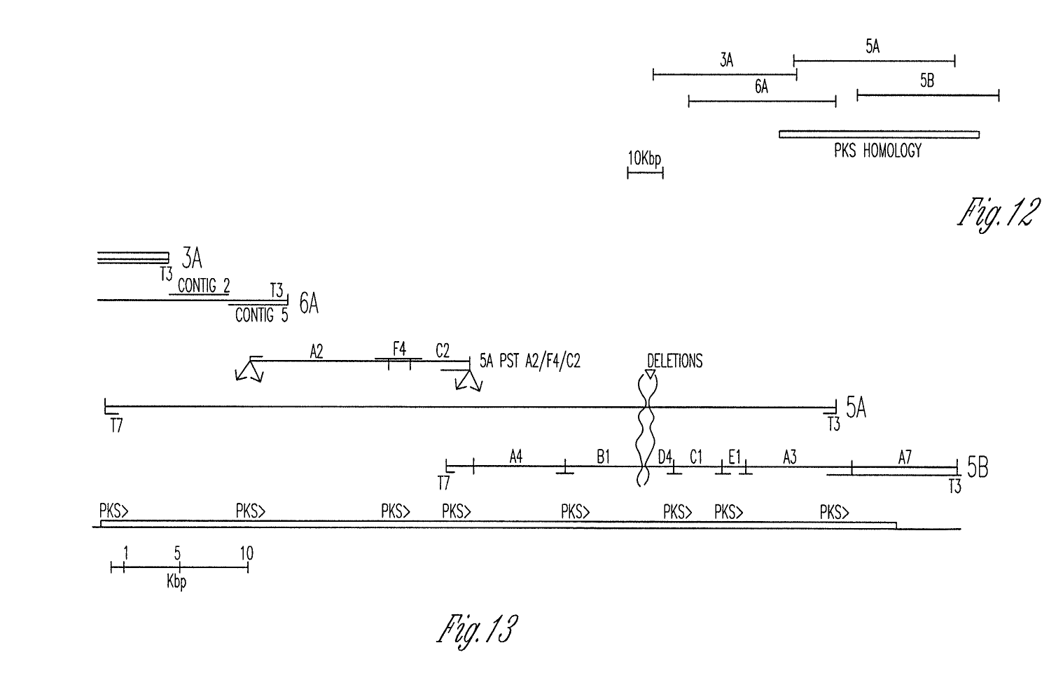

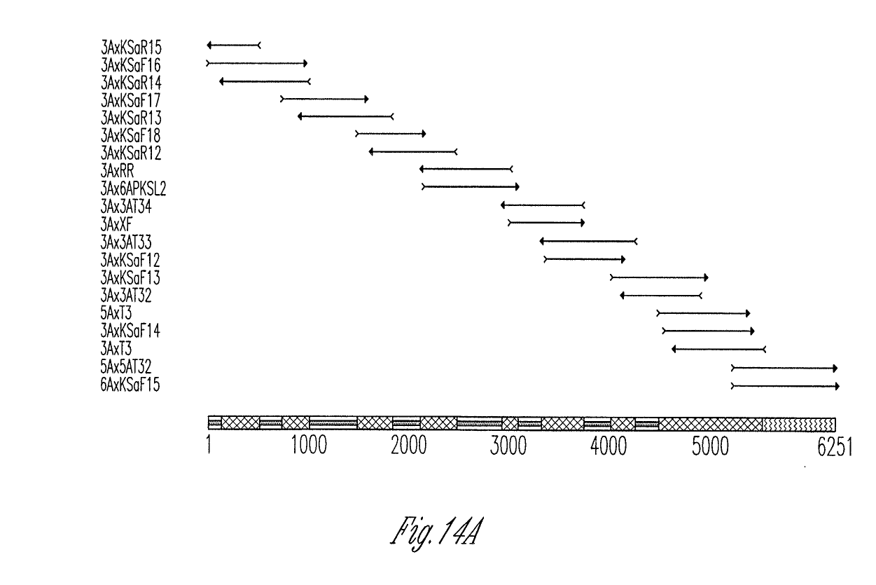

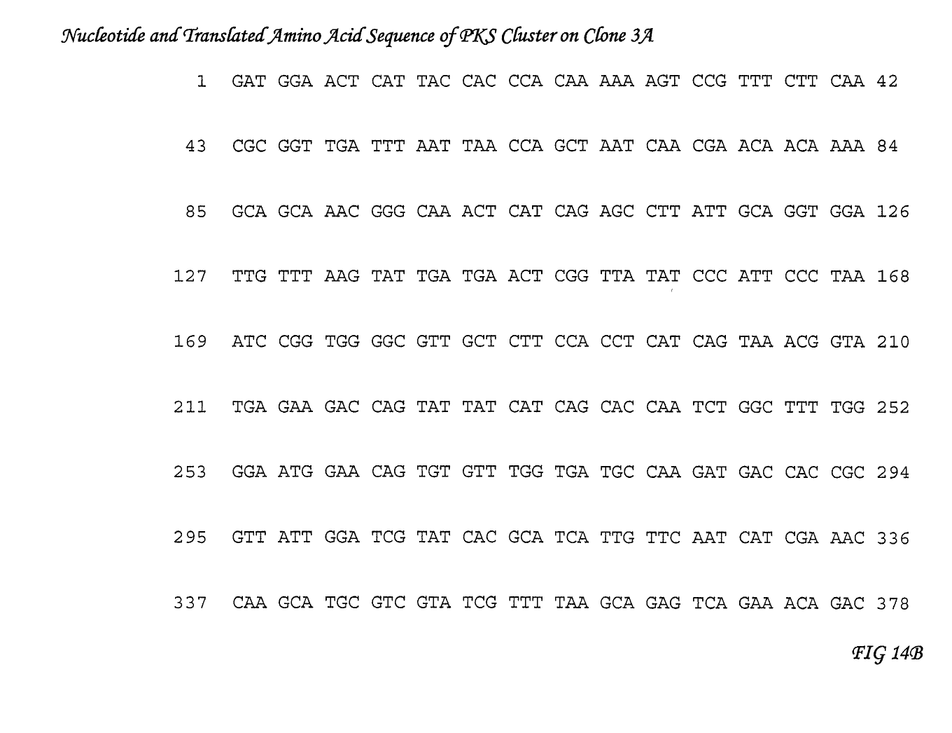

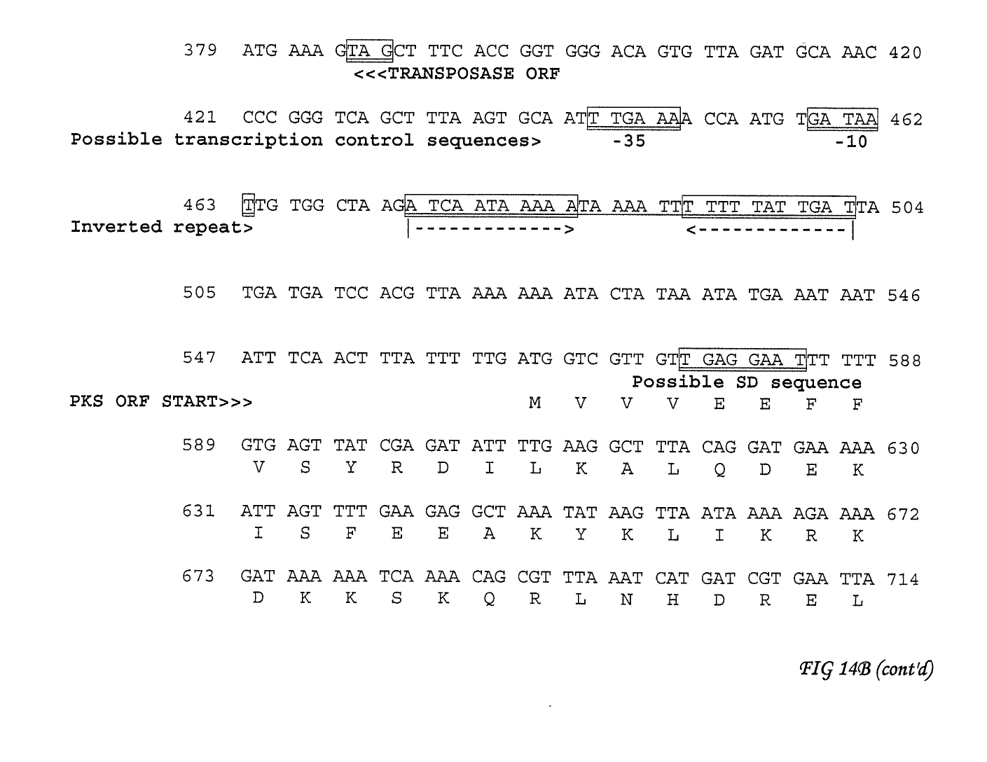

A probe was derived from a restriction fragment at the T3 end of 6A and used to rescreen the cosmid library. Twenty-seven additional colonies were isolated. After characterization by restriction digests and hybridization using the KSa probe, clones 5A and 5B were shown to contain multiple EcoRI/SalI fragments with KSa homology (FIG. 11). An overall map for clones 3A, 6A, 5A, and 5B are shown in (FIG. 12). DNA preparations of clones 5A and 5B were sequenced from the T3 and T7 ends. For clone 5A, PKS homology was identified at both ends, and the predicted direction of transcription from the T7 to the T3 end suggested that the entire insert (approx. 35 kbp) contained PKS homology. For clone 5B, PKS homology was identified at the T7 end, and homology to glutathione reductase at the T3 end.

Subcloning and Sequencing of Cosmid Clones

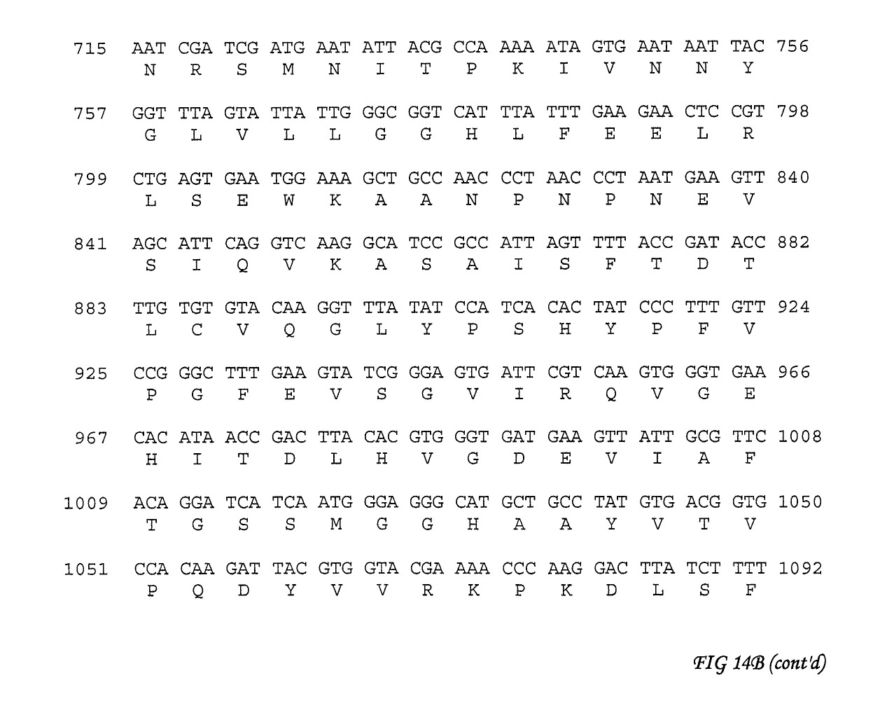

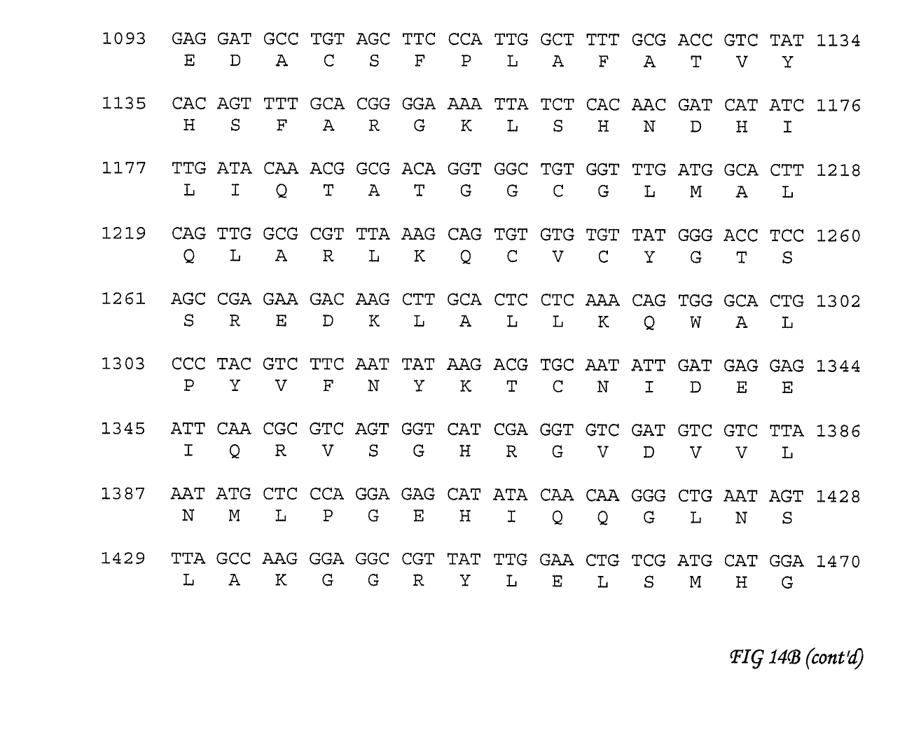

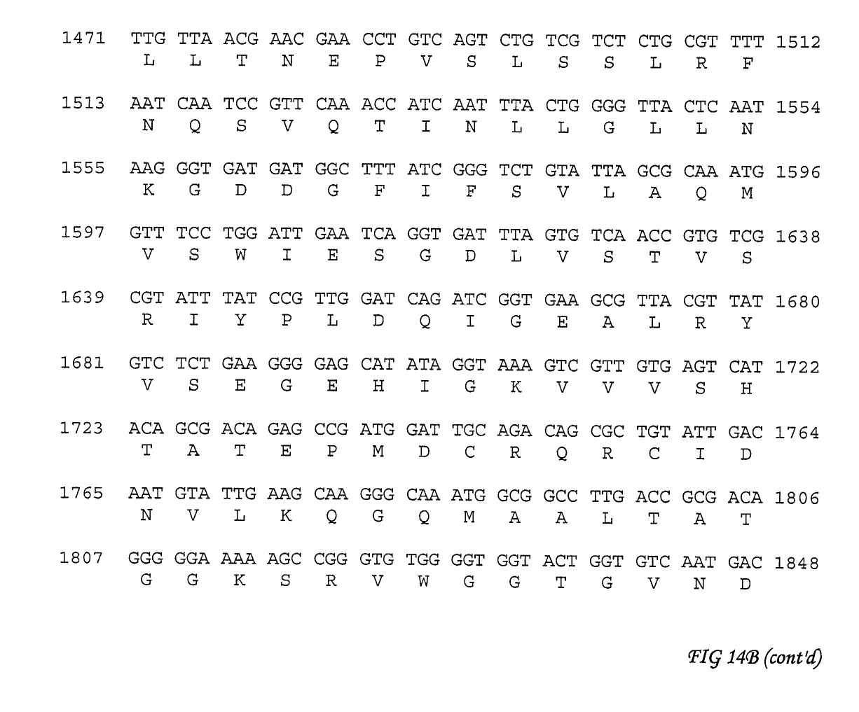

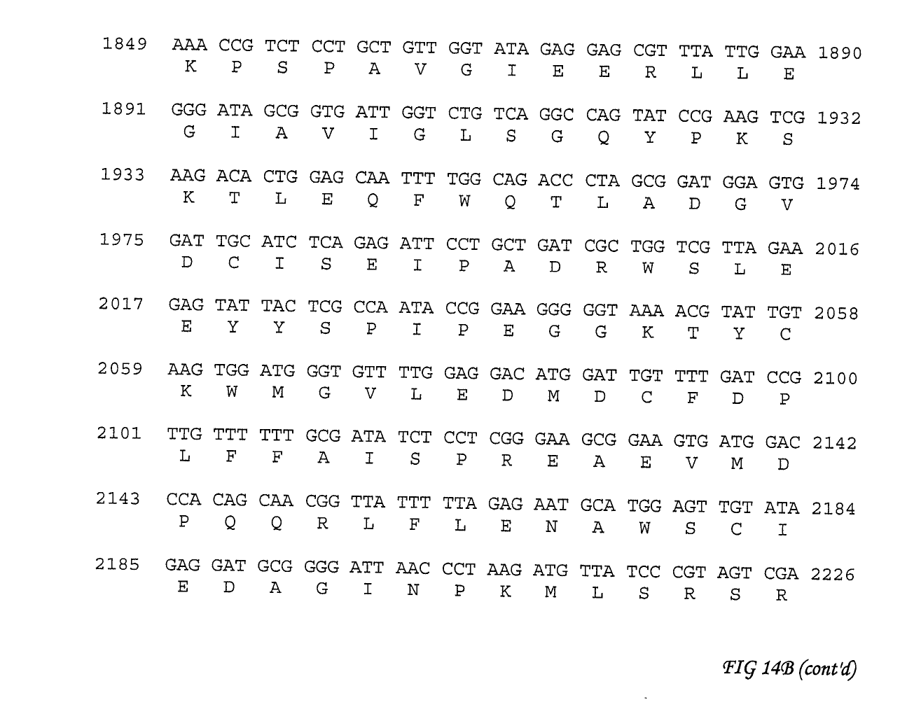

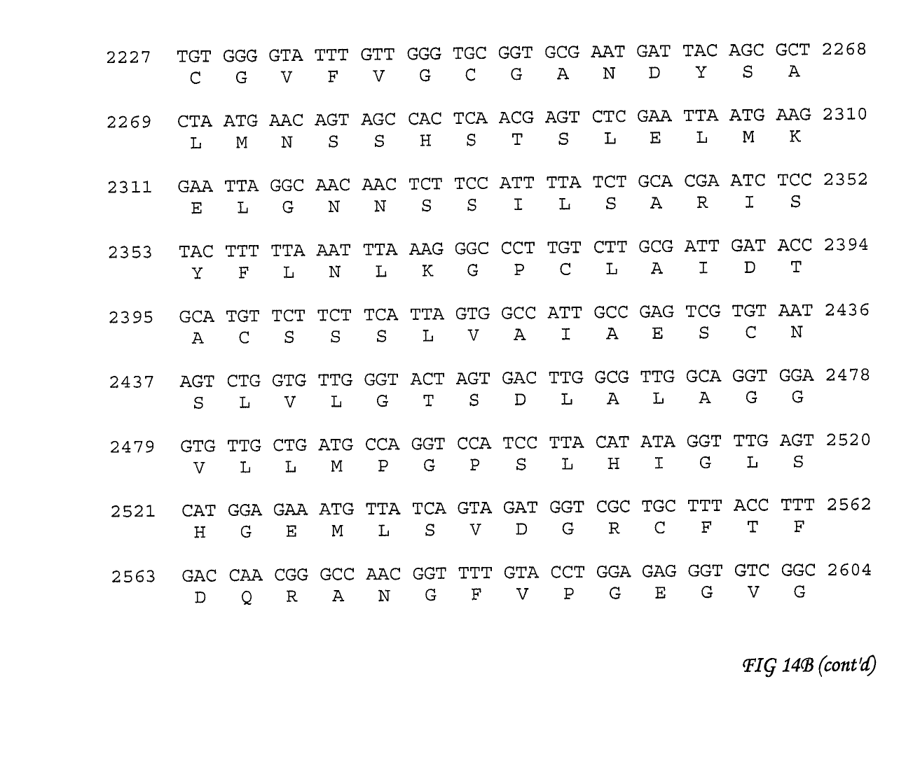

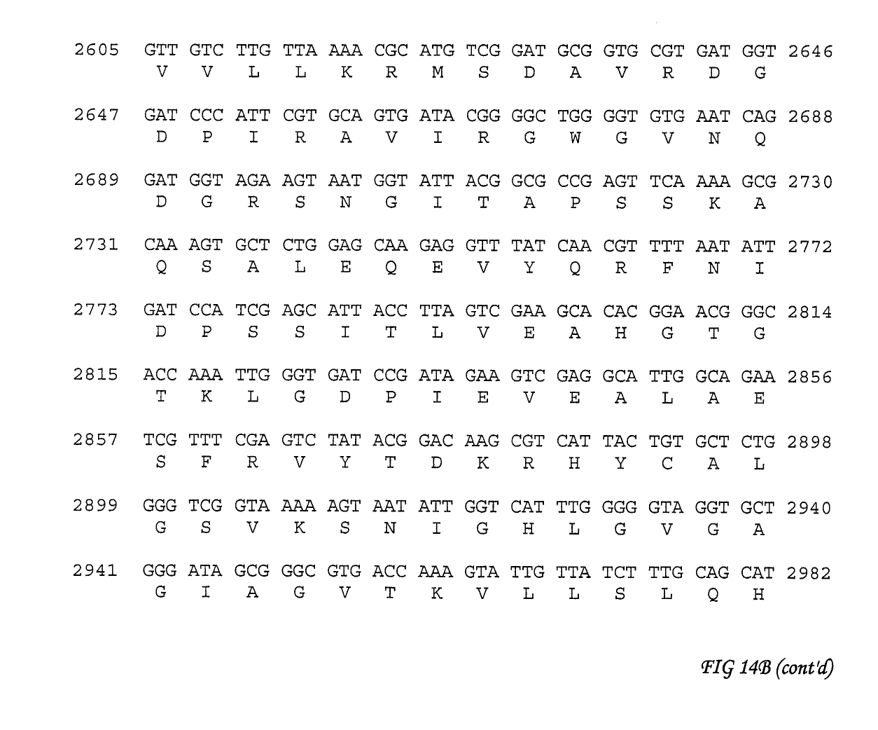

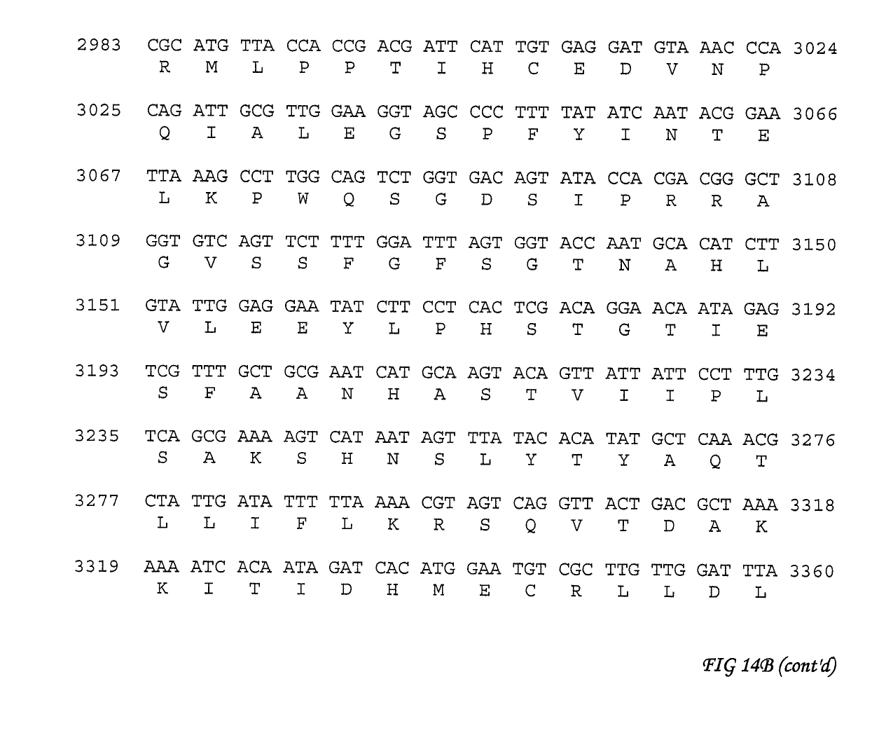

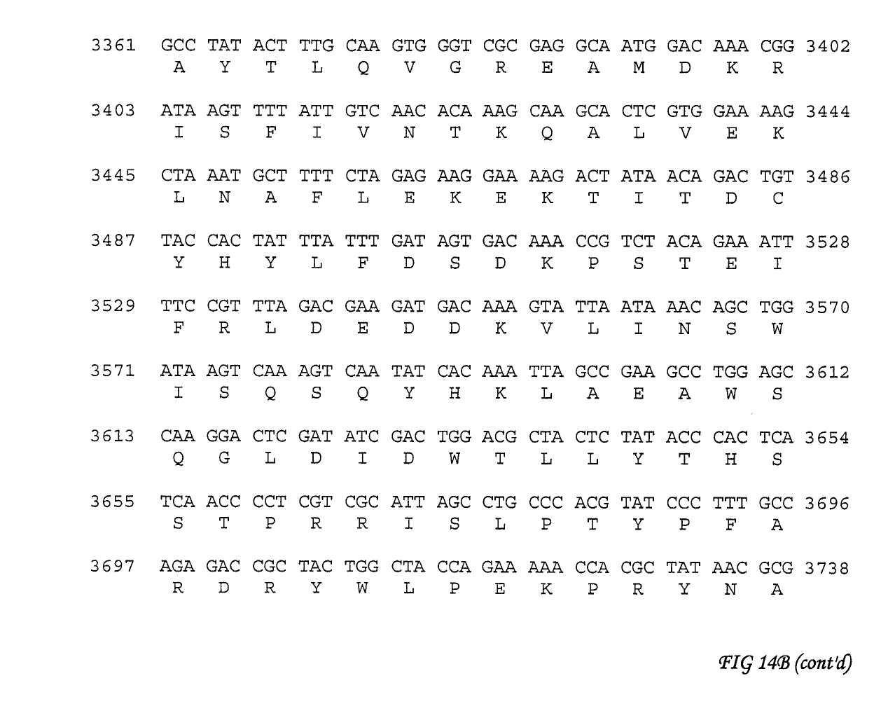

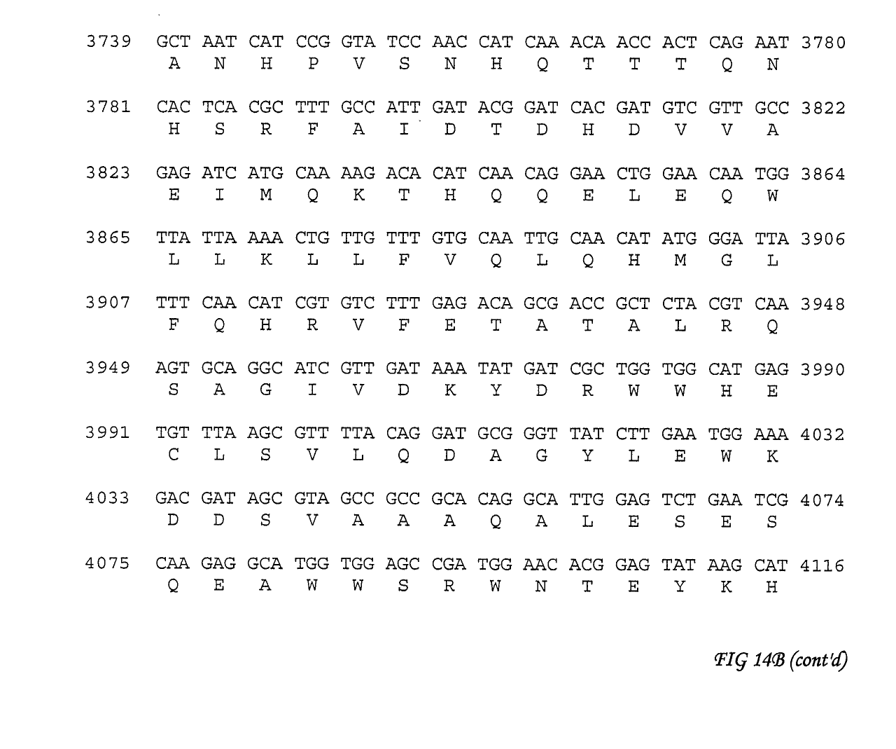

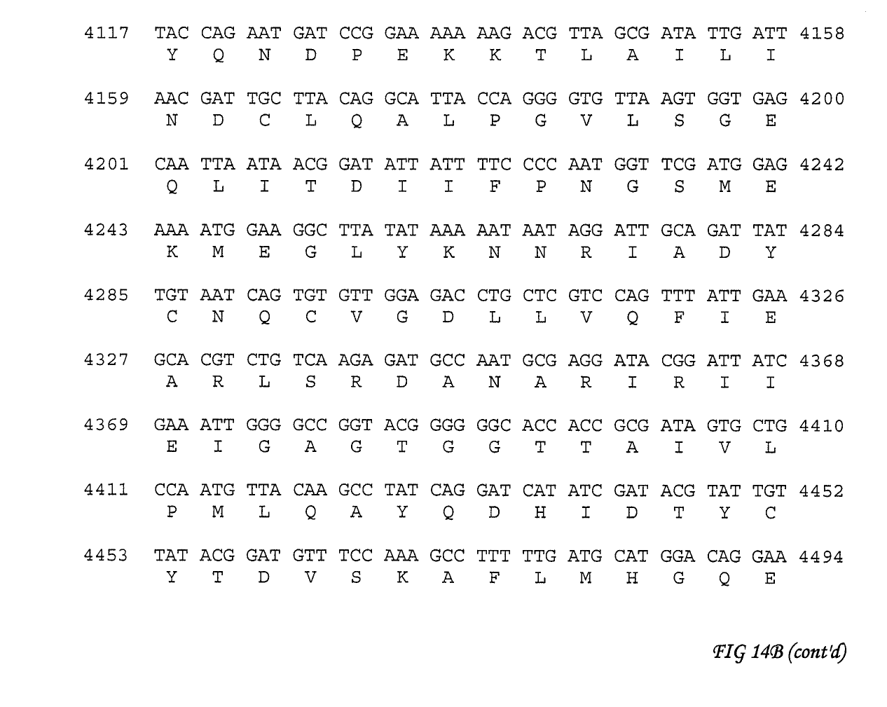

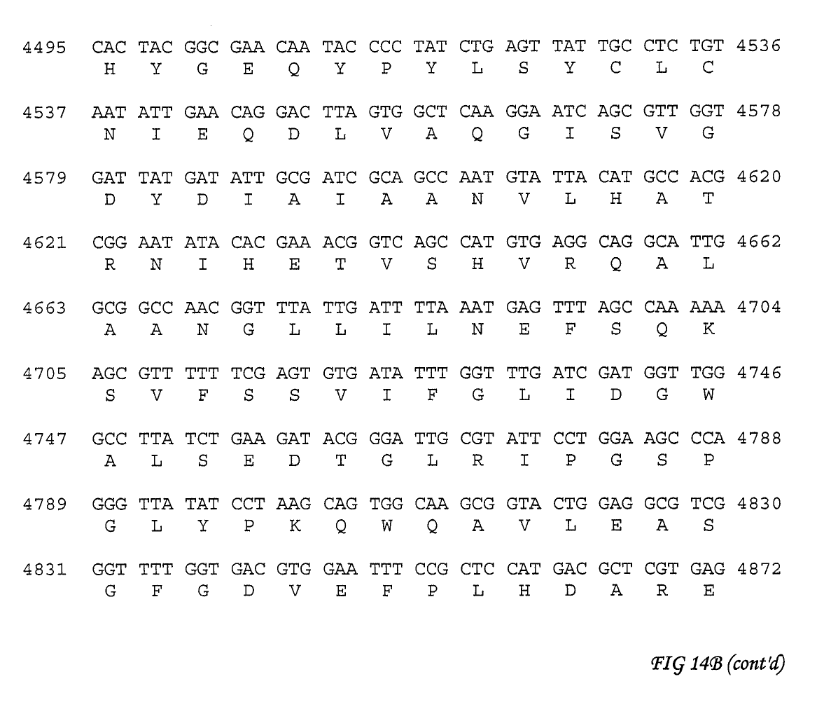

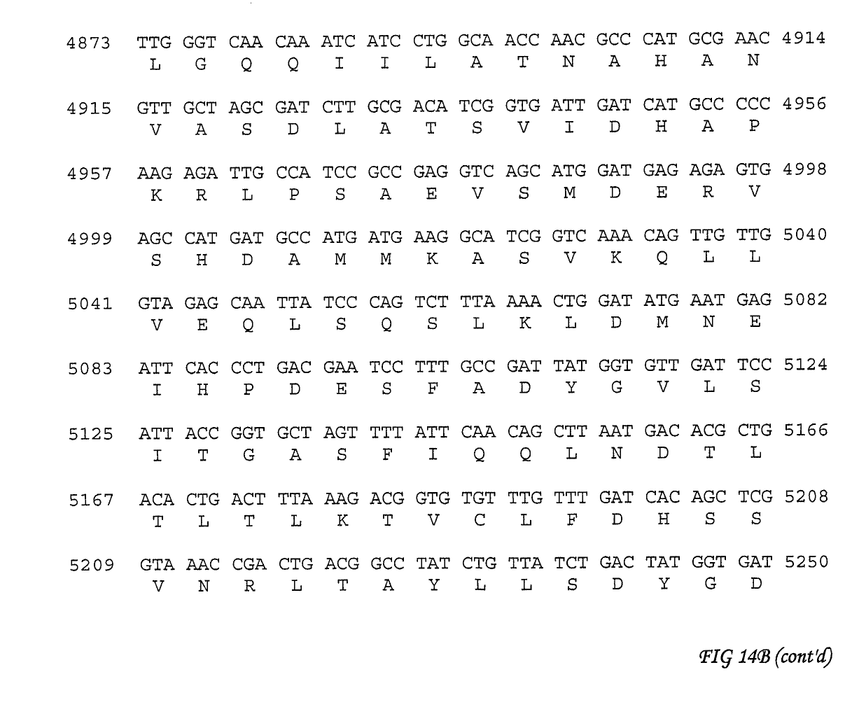

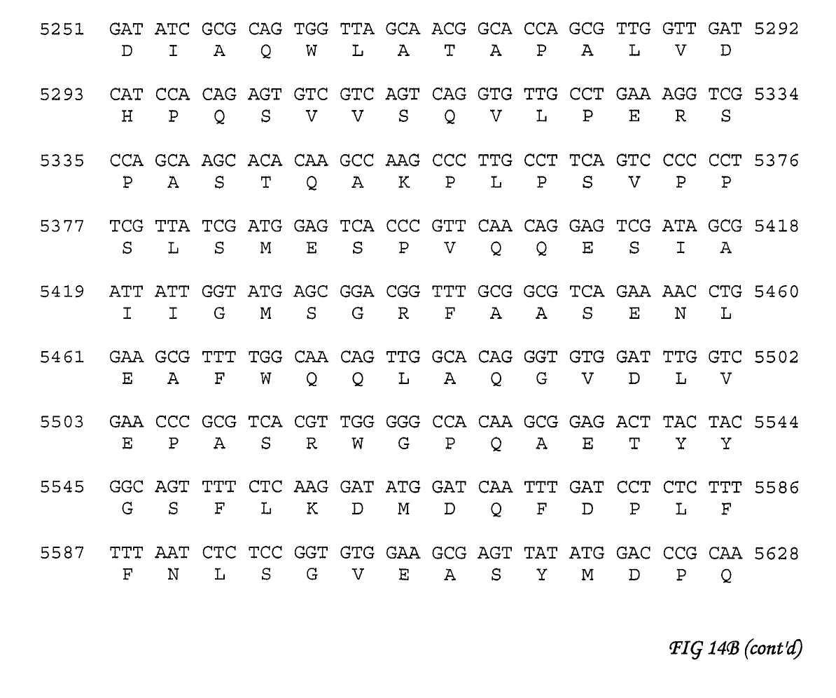

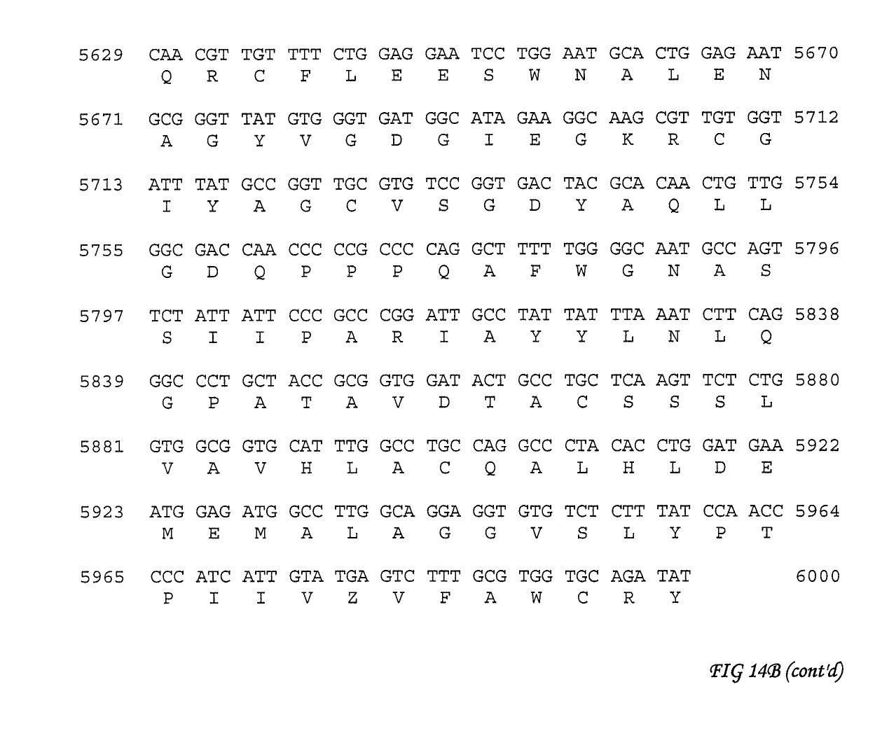

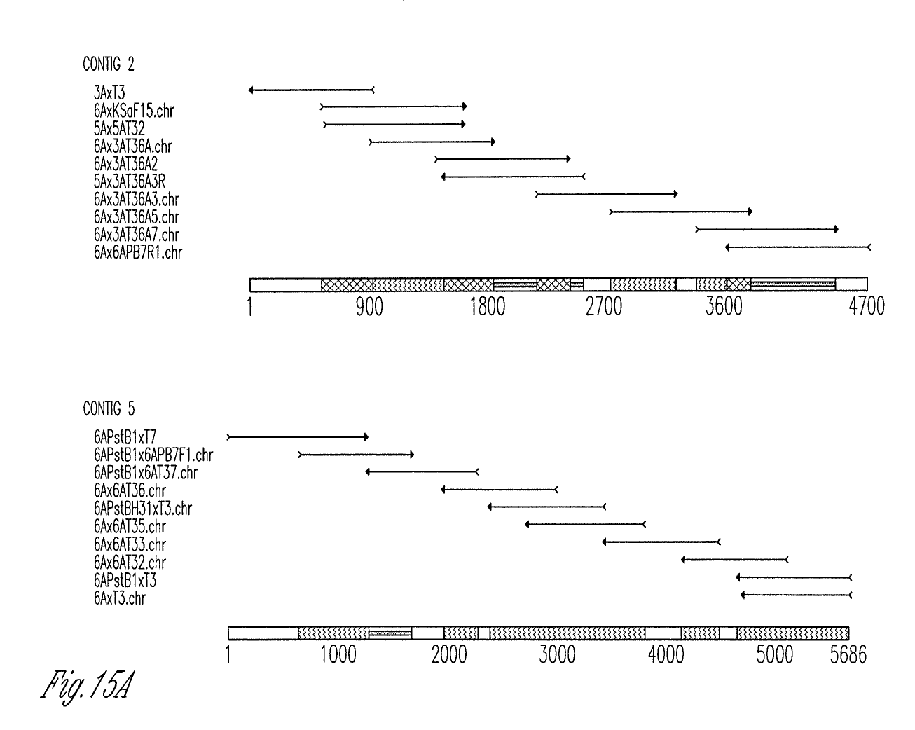

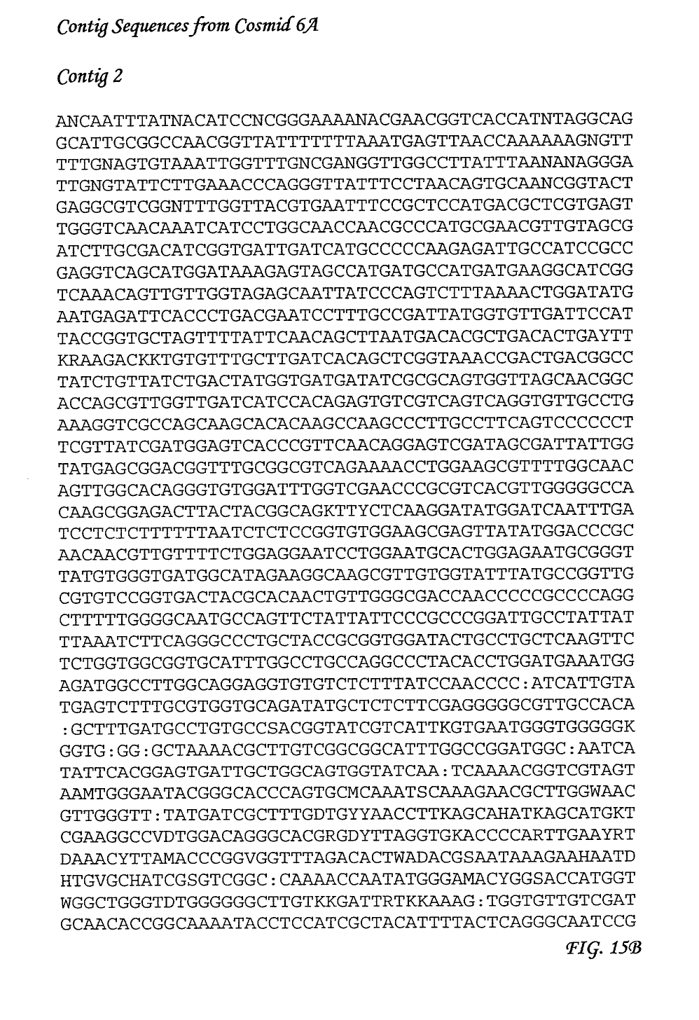

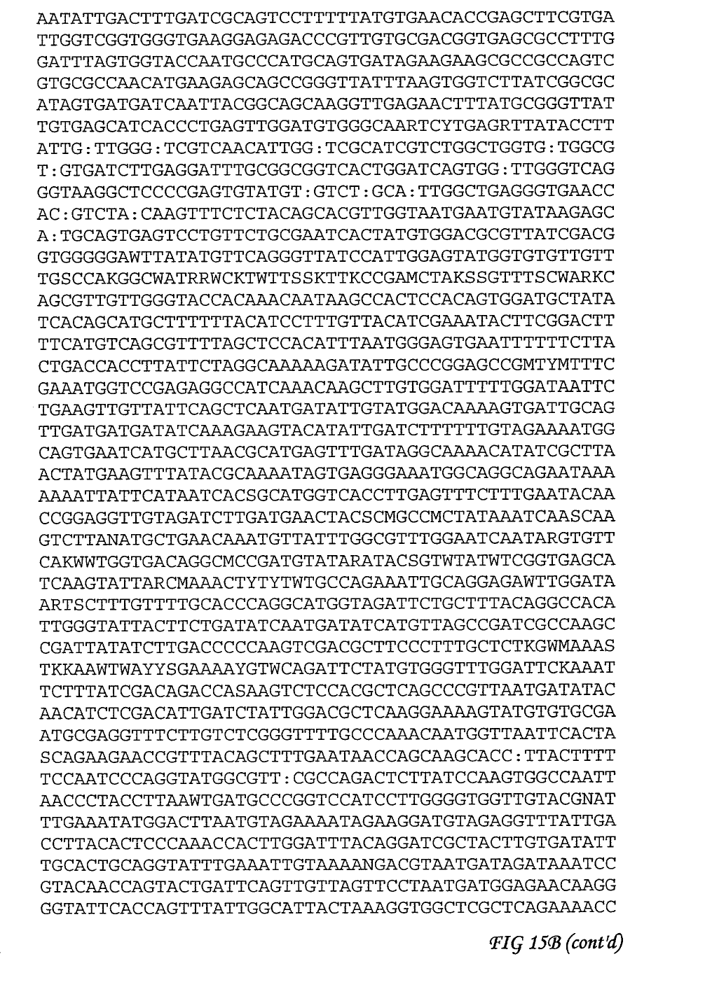

Detailed maps of the cosmid clones and regions sequenced are presented in FIG. 13. Clone 3A was sequenced from the T3 end, and from both directions in the KSa region by designing oligonucleotide primers to extend existing sequence until sequences overlapped (FIG. 14). All primers for this project were from Integrated DNA Technologies, Inc. (Coralville, Iowa). The putative start of the PKS cluster was identified approximately 5.5 kbp from the T3 end of 3A, and this was preceded by an open reading frame with homology to a transposase. No Shine-Delgarno sequence is observed upstream of the putative start site whereas a possible Shine-Delgarno sequence appears downstream of the ATG site and may be indicative of the start site being several codons further downstream of the putative start. The region between the transposase and the PKS cluster is likely to contain control elements for transcription of the cluster and possible elements are identified [SEQ ID NO:29].

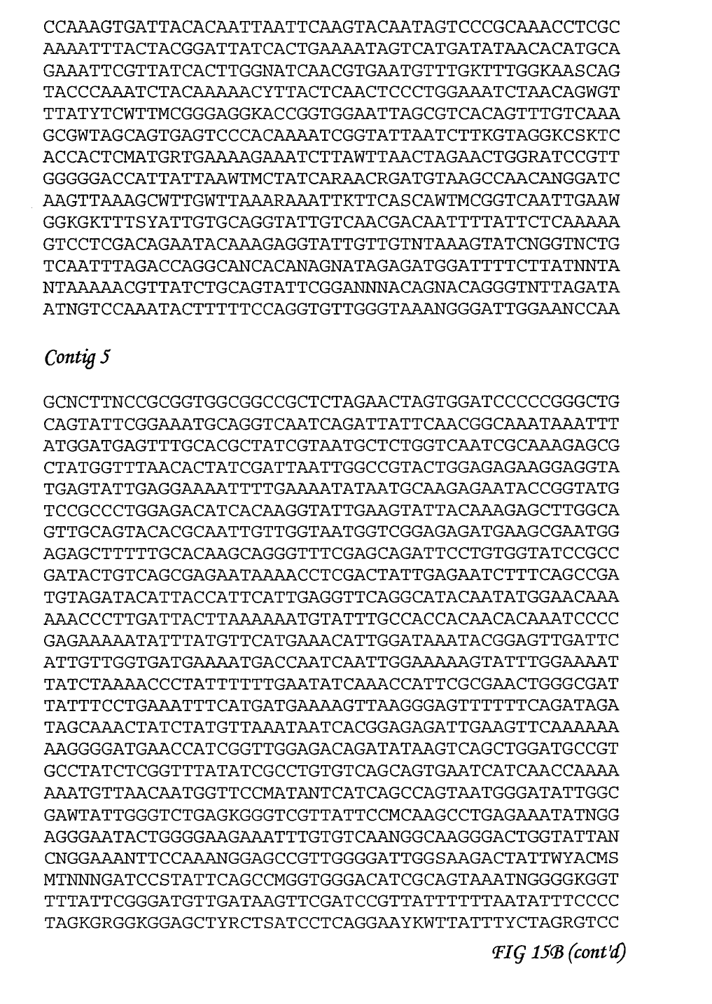

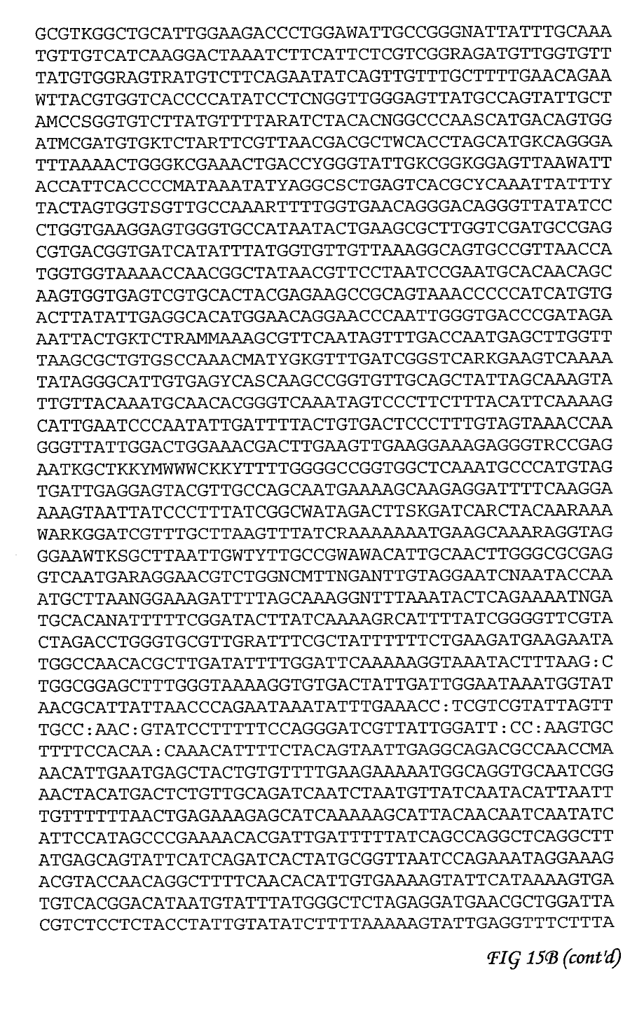

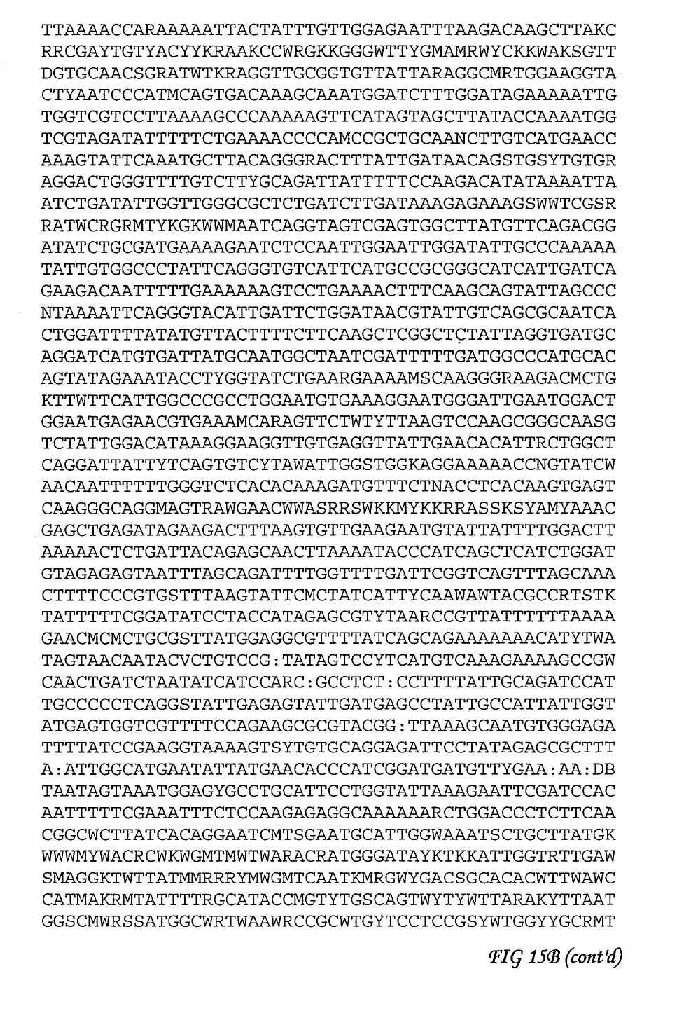

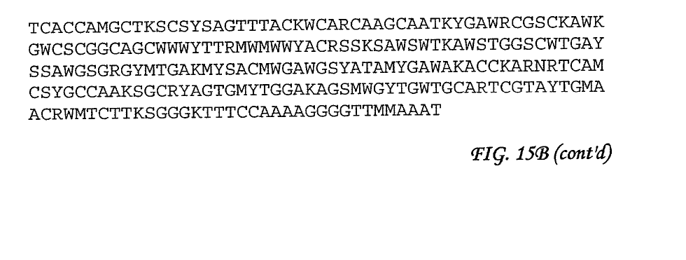

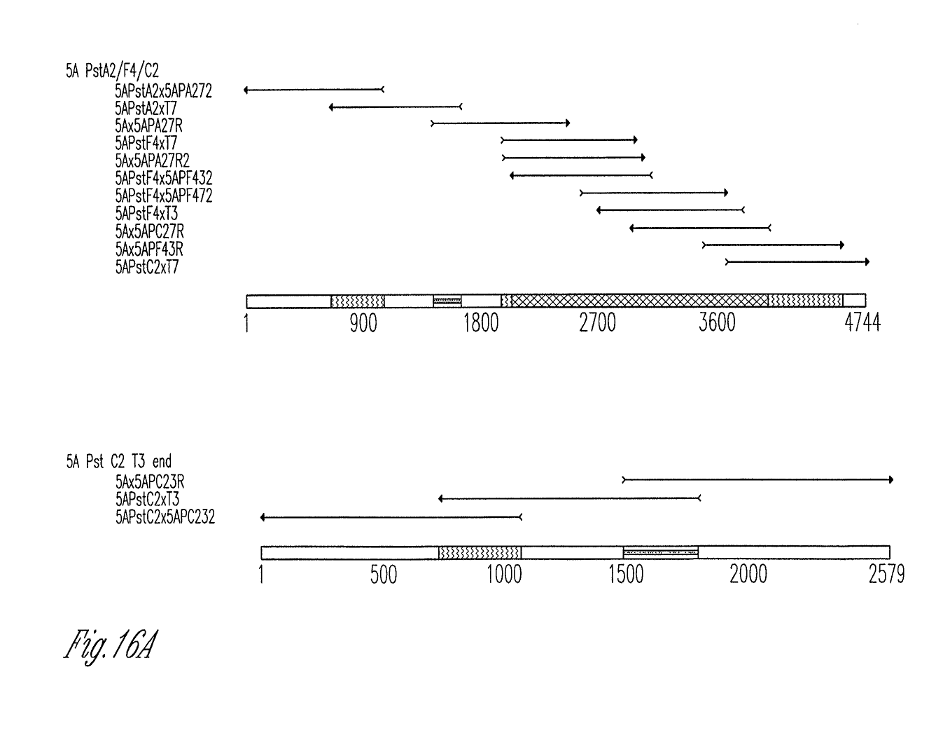

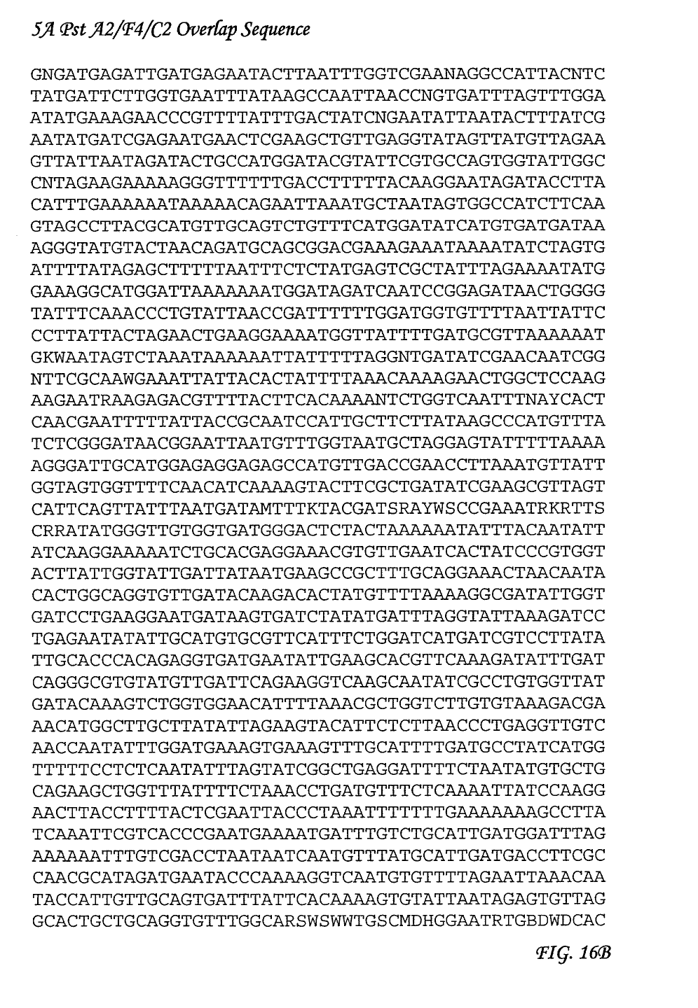

The portion of clone 6A downstream from 3A was sequenced, and upstream from its T3 end. The sequencing strategy of this region is presented in FIG. 15 with the sequence of a small gap yet to be determined and therefore the sequence of this region of clone 6A is presented as two contigs, No. 2 and No. 5 (FIG. 13, SEQ ID NO:30 and SEQ ID NO:31 respectively).

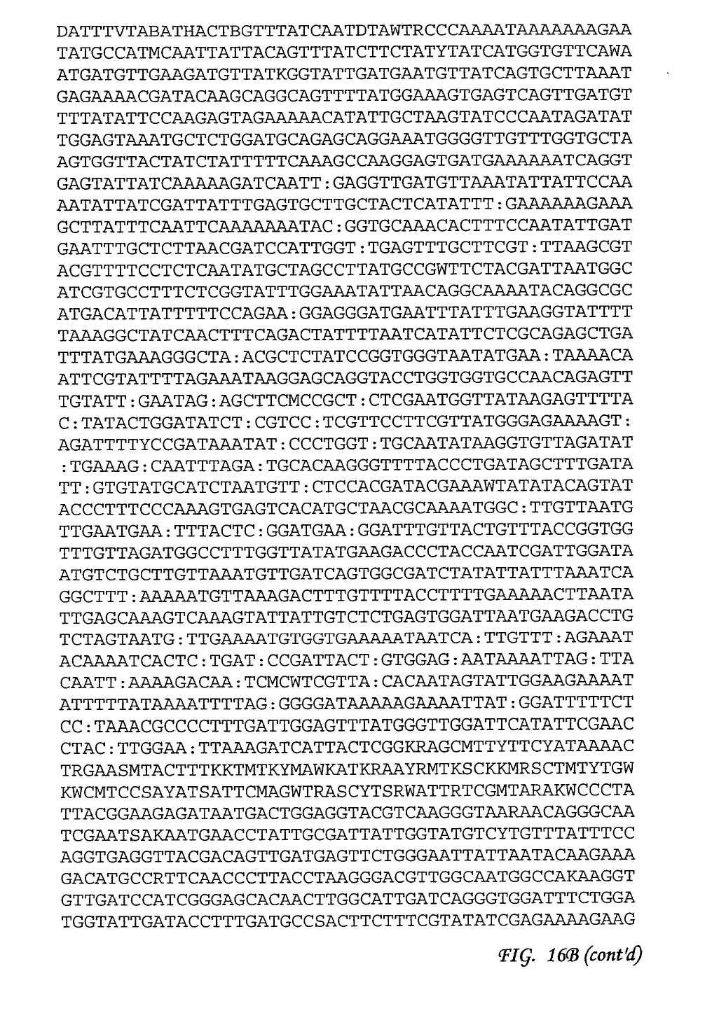

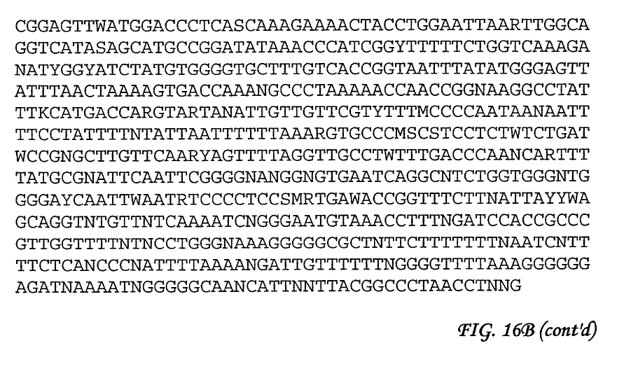

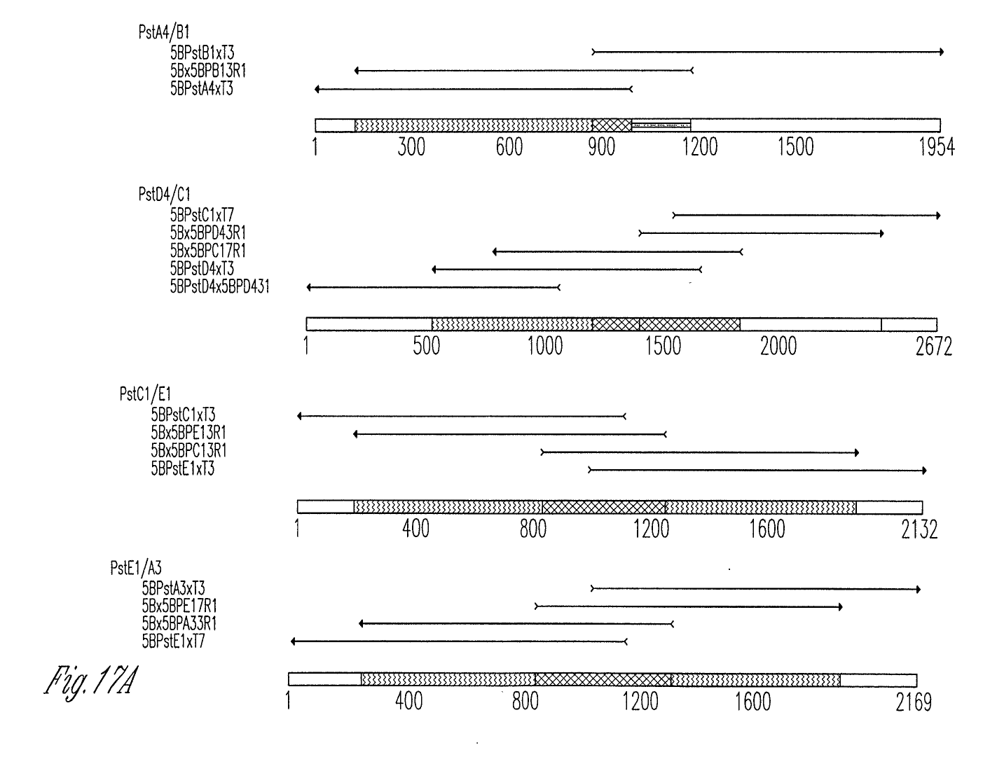

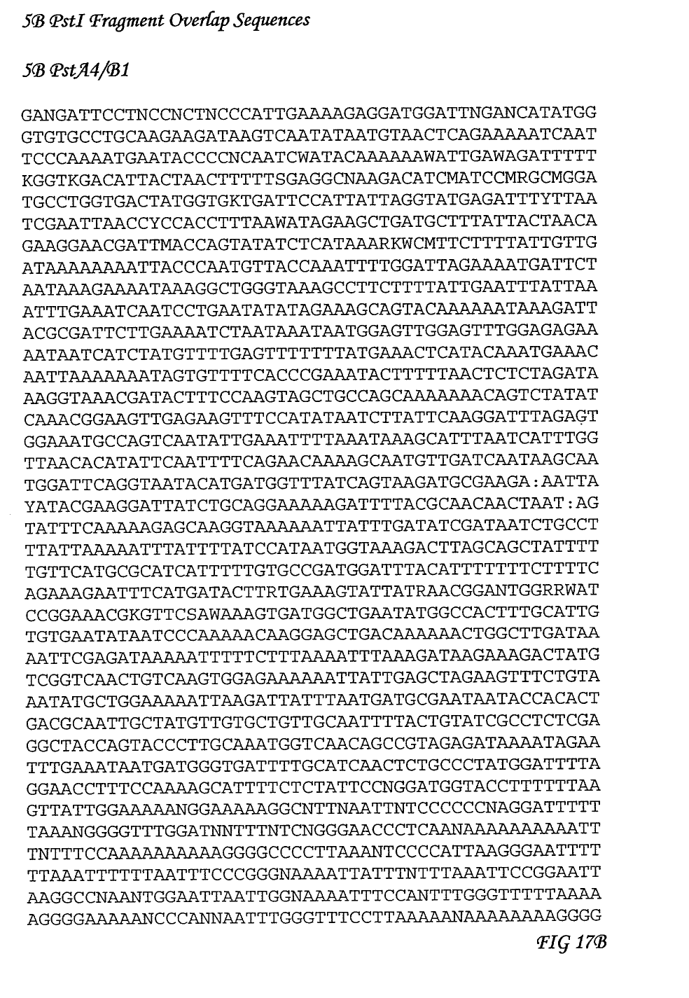

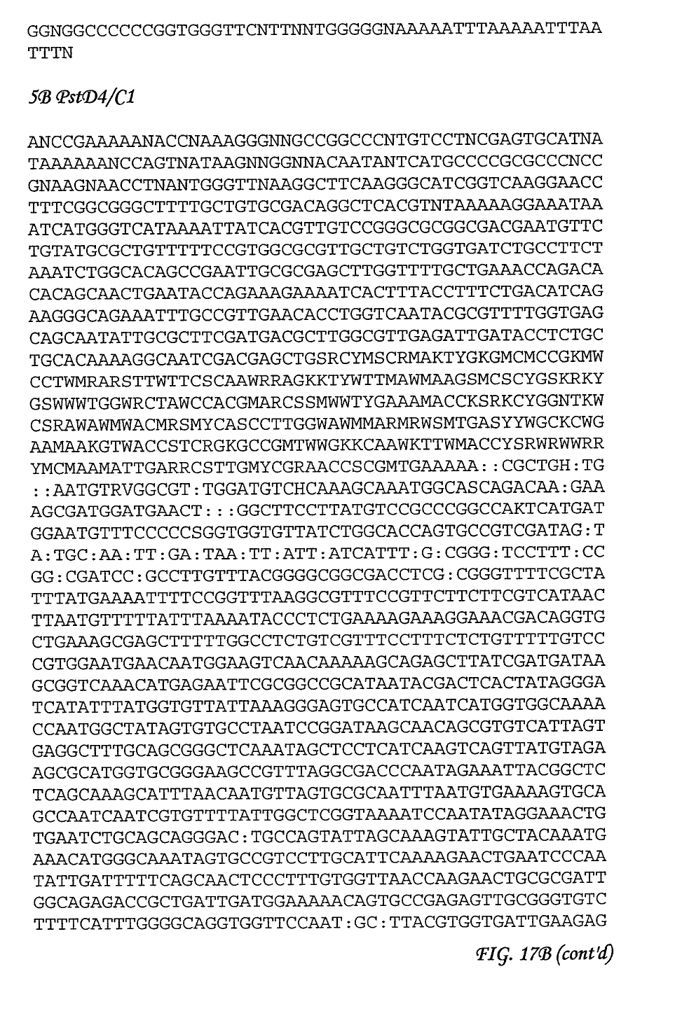

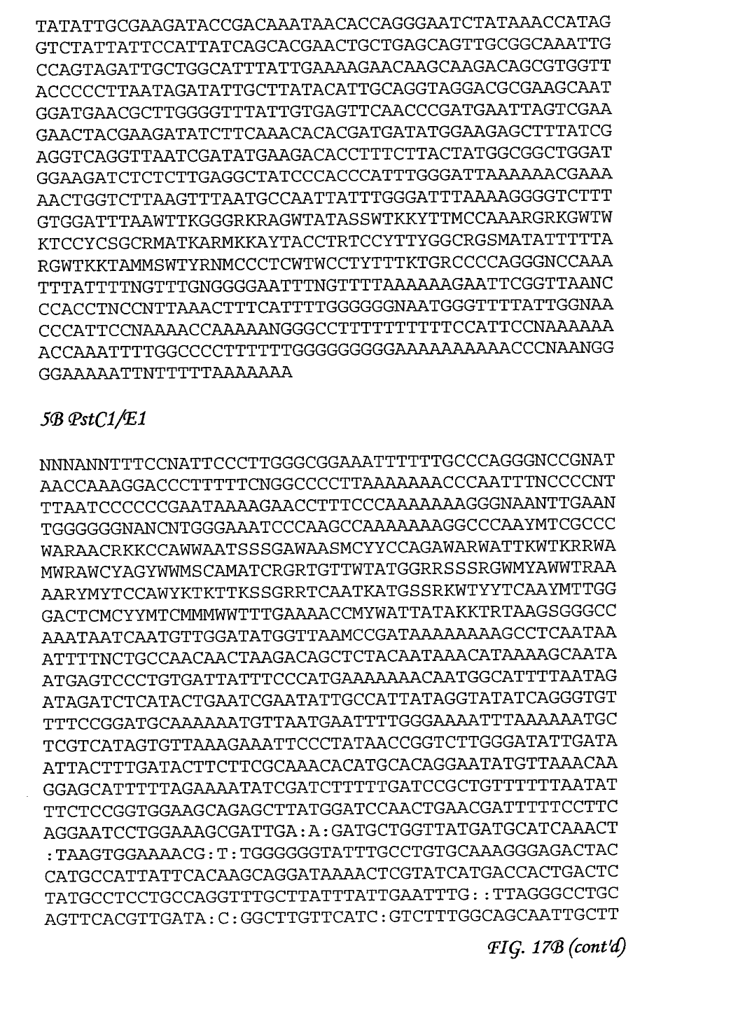

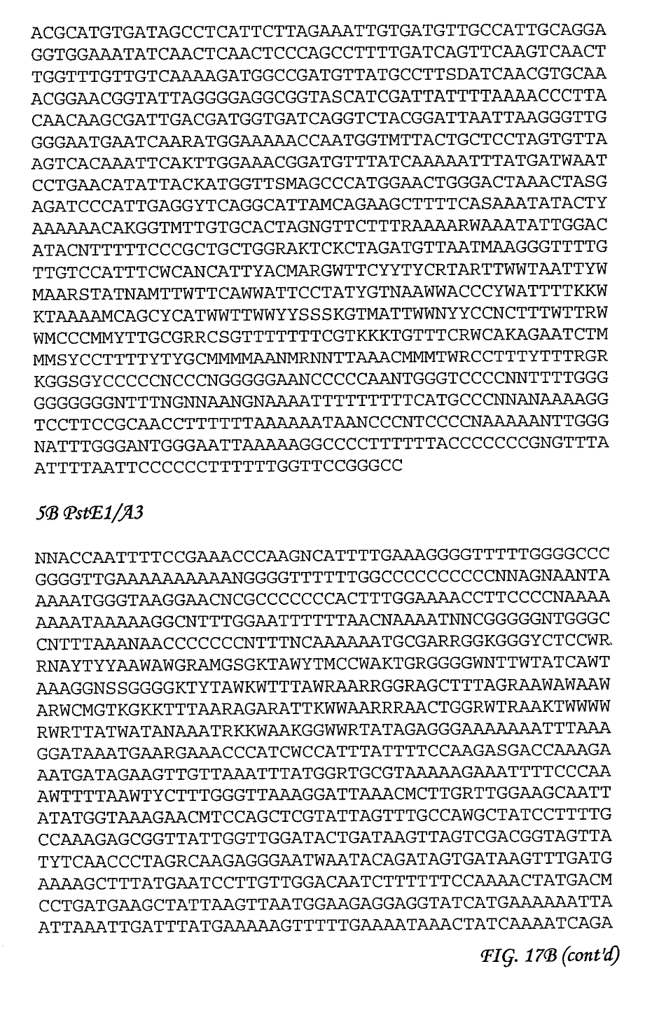

PstI fragments of clones 5A and 5B (FIG. 13) were subcloned into a pBluescript vector and sequenced. Each fragment in 5B was sequenced from its T3 and T7 ends, and fragments in 5A that did not overlap with these were sequenced similarly. From this sequence, primers were designed to sequence in the opposite direction on the cosmid template to determine which restriction fragments were adjacent to each other. In 5A, it was determined that PstI fragments A2, F4, and C2 overlapped (FIG. 13 and FIG. 16, and SEQ ID NO:32). These sequences have not been precisely located on the map but they are believed to be in the general area as shown in (FIG. 13).

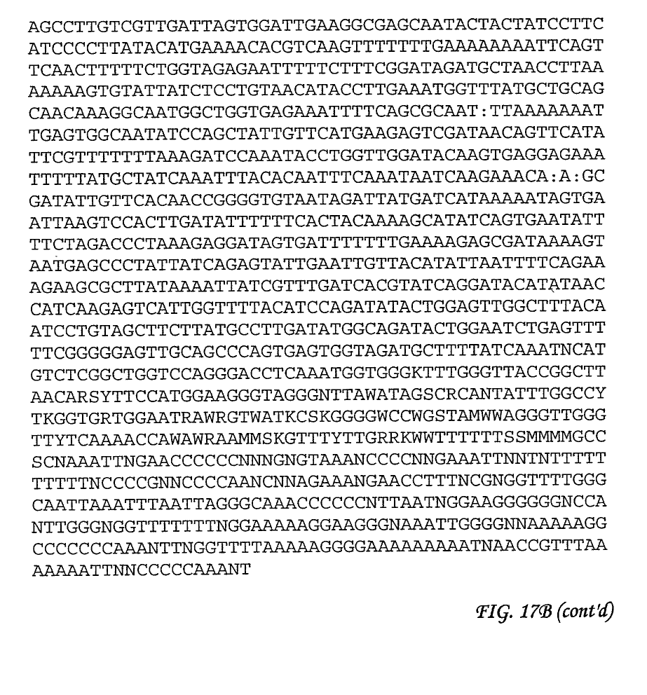

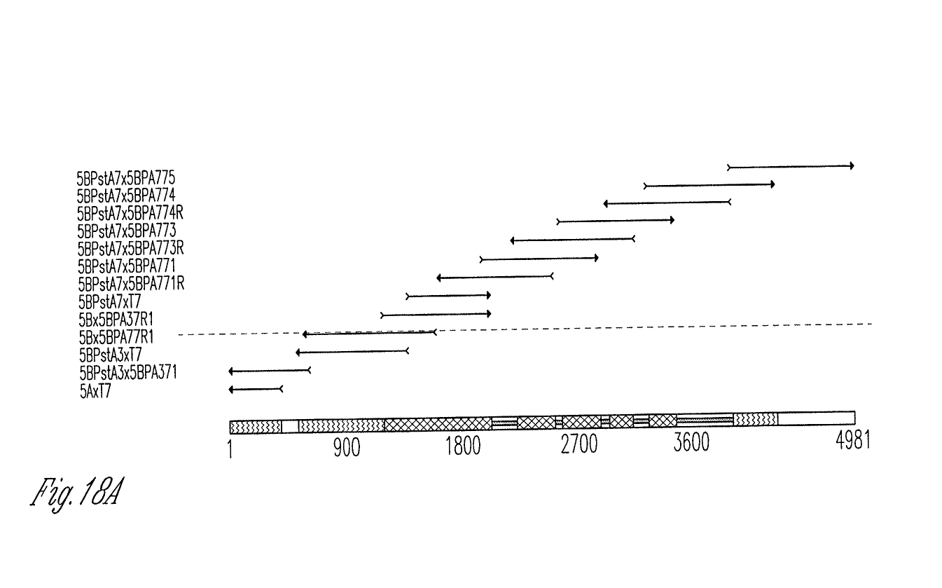

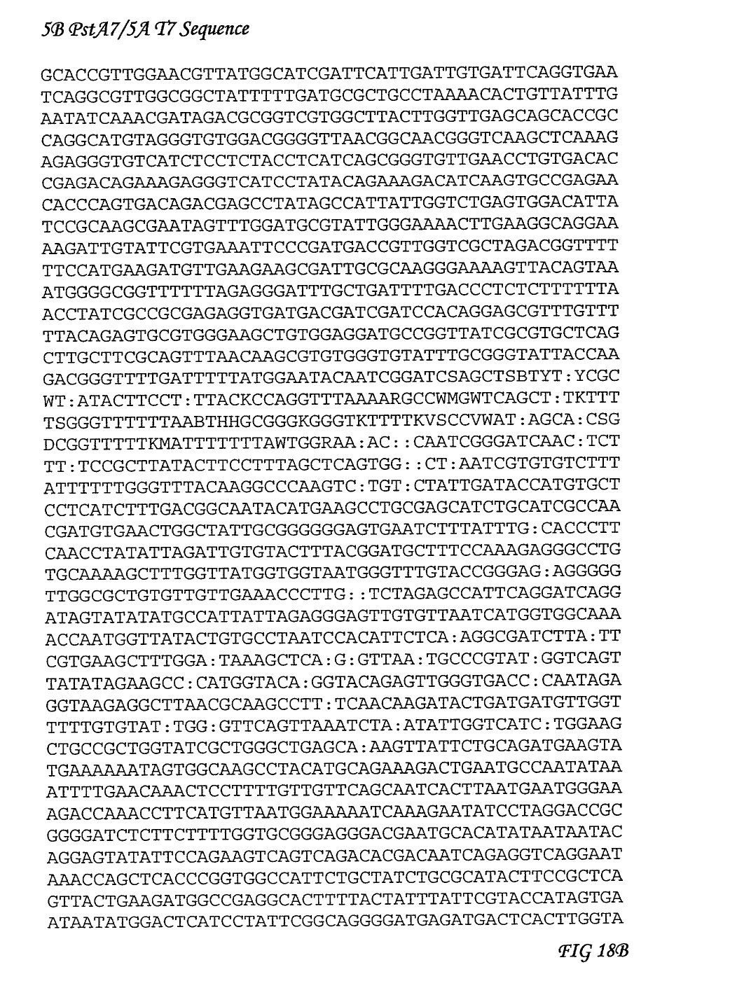

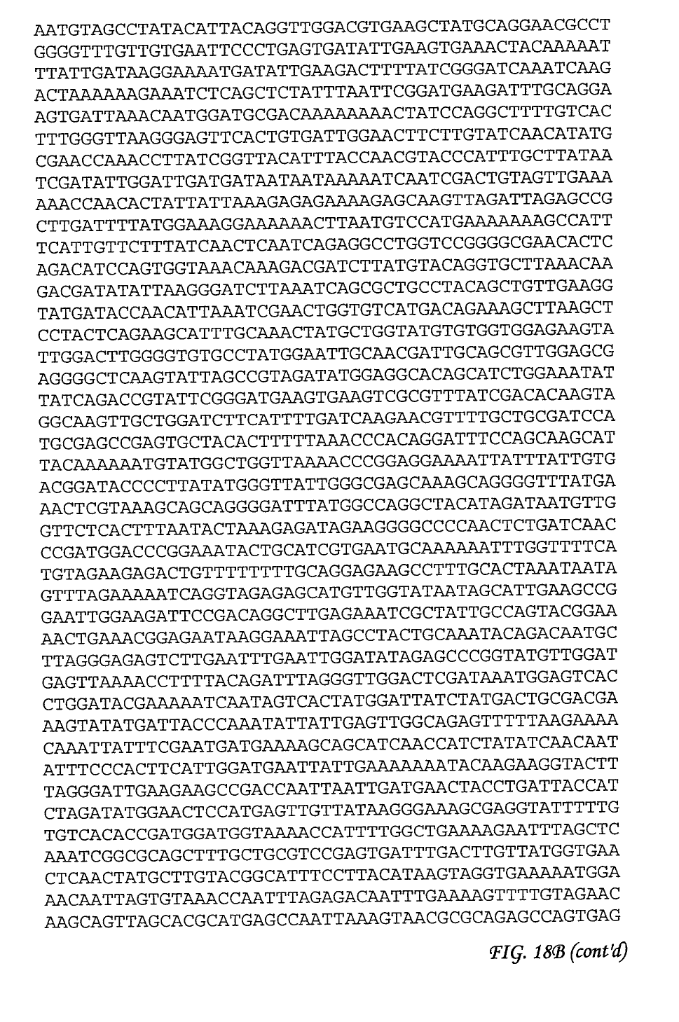

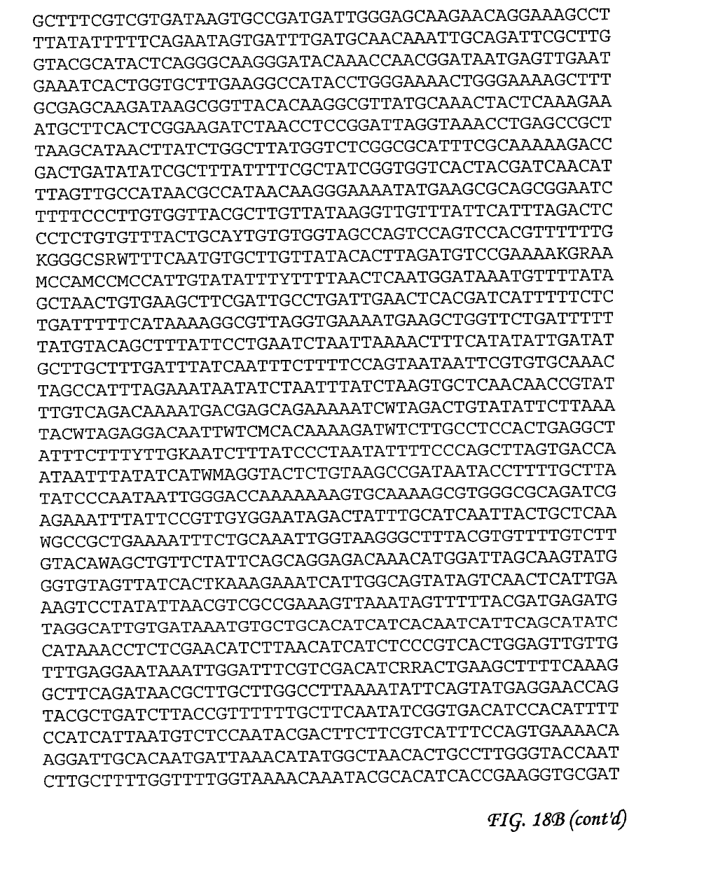

For clone 5B, PstI fragments A4 and B1 overlap, as well as PstI fragments D4 and C1, C1 and E1, E1 and A3, and A3 and A7 (FIG. 13 and FIG. 17). Overlapping sequences are presented for the PstI subcloned fragments of clone 5B; PstA4/B1 (SEQ ID NO:33), PstD4/C1 (SEQ ID NO:34), PstC1/E1 (SEQ ID NO:35), and PstE1/A3 (SEQ ID NO:36). Overlapping sequences between PstI fragments B 1 and D4 could not be identified, and with subsequent results, suggests that a portion of the cosmid clones may have been deleted in this region. The entire sequence of 5B PstA7 has been determined on at least one strand (FIG. 13 and FIG. 18; SEQ ID NO:37) and the end of the PKS cluster has been identified in this fragment, approximately 3,200 bp from the end of A7 nearest the T3 end of 5A.

The locations of all regions where PKS homology was identified by sequence analysis are presented in (FIG. 13). PKS homology extends throughout the region sequenced, and our current estimate of the total length of the cluster is 52-56 kbp, large enough to encode enzymes to synthesize bryostatin. To date approximately 38,000 bp of unique sequences have been identified in this region.

TABLE IV
Sequences of clones B. neritina and predicted amino acid product of
SEQ ID NO: 29

| Description | SEQ ID NO |
|---|---|
| Nucleotide sequence of PKS cluster on clone 3A | SEQ ID NO: 29 |
| Contig 2 sequences from cosmid 6A | SEQ ID NO: 30 |
| Contig 5 sequences from cosmid 6A | SEQ ID NO: 31 |
| Cosmid clone 5A Pst A2/F4/C2 overlap sequence | SEQ ID NO: 32 |
| Cosmid clone 5B Pst A4/B1 overlap sequence | SEQ ID NO: 33 |
| Cosmid clone 5B Pst D4/C1 overlap sequence | SEQ ID NO: 34 |
| Cosmid clone 5B Pst C1/E1 overlap sequence | SEQ ID NO: 35 |
| Cosmid clone 5B Pst E1/A3 overlap sequence | SEQ ID NO: 36 |
| Cosmid clone 5B Pst a7/5A T7 sequence | SEQ ID NO: 37 |
| Predicted amino acid product of SEQ ID NO: 29 | SEQ ID NO: 38 |

### Example 5

### Combinatorial Biosynthesis

Combinatorial biosynthesis has generally been used in the search for novel molecules with applications as pharmaceuticals or as platforms for combinatorial synthesis. For example, Shen et al. have demonstrated that engineered aromatic or modular PKSs can be used to generate polyketide libraries of different molecular sizes and shapes (Shen et al., 1999). The biosynthetic genes of the present invention, for example for bryostatin synthesis, could be incorporated into these systems to create derivatives/analogs of bryostatins with improved properties such as reduced toxicity/myalgia, greater efficacy etc. (Shen et al., 1999; Xue et al., 1998). Recently, the erythromycin PKS genes have been engineered to effect combinatorial alterations of catalytic activities in the biosynthetic pathway (McDaniel et al., 1999). This has resulted in the successful generation of more than fifty macrolides which would otherwise be impractical to produce through chemical methods. This leads to the creation of libraries of novel “unnatural” natural products exhibiting altered functions (McDaniel et al., 1999). The bryostatin PKS genes could be used in such a system to create analogs of bryostatin with improved properties.

The cloned biosynthetic genes presented here have applications in bioprospecting. The cloned PKS genes could be used in PCR, in situ hybridizations, etc to isolate novel marine (and terrestrial) PKS and polyketides which may exhibit novel structures and novel activities (antibacterial, antifungal, anticancer, etc.)

Due to the novel GC content of the biosynthetic clones presented, these clones have application in screening molecular diversity from environmental DNA samples to identify novel PKS genes on the basis of this low GC content. In high stringency hybrizations, these very low GC PKS gene fragments could be used as probes to detect novel PKS's from environmental isolates, heretofore unknown since the current approach in the field is to use Streptomyces PKS genes as probes which are high GC and would possibly not hybridize to our gene (s) (and other members of this proposed gene family) due to its lower GC content (Schupp et al., 1995). The clones presented herein thus represent a novel class of low GC content PKSs. Precedent exists for expression of PKS in nonpolyketide-producing prokaryotic and eukaryotic hosts (Kealey et al., 1998).

### Example 6

### PKS Genes from B. Pacifica

DNA Extraction

Adult B. pacifica DNA was extracted using a QIAamp Tissue Kit (Qiagen Inc., Valencia, Calif.) as described for the extraction of the B. neritina larval DNA.

PCR Conditions for the Amplification of SSU rRNA from B. pacifica

The PCR conditions for the initial amplification of the B. pacifica ribosomal small-subunit (SSU) rRNA gene sequences were as follows. E. sertula specific 16S rRNA PCR primers were used as previously described (Haygood, 1997). A total reaction volume of 50 microliters contained approximately 200 ng of adult B. pacifica DNA, 1 millimolar each 16s SSU rRNA primer (198F and 1253R), and Taq polymerase and buffer (Boehringer Mannheim Corp., Indianapolis, Ind.). PCR conditions for the SSU rRNA primers were as follows: denaturation (94 C; 1 min), annealing (54 C; 1 min), and extension (72 C; 1 min) for 30 cycles.

Conditions for the PCR amplification of KS DNA from B. pacifica (using the degenerate primers PKSR and BLCASPKS) were identical to those used for B. nertina.

| Primer | | |
|---|---|---|
| Designation | | |
| and | | |
| Orientation | Sequence | SEQ ID NO: |
| 240F (forward) | TGC TAT TTG ATG AGC CCG CGT T | SEQ ID |
| | | NO: 7 |
| 1253R | CAT CGC TGC TTC GCA ACC C | SEQ ID |
| (reverse) | | NO: 8 |

Sequences isolated from B. pacifica using these primers are provided in SEQ ID NO: 13 and SEQ ID NO: 15. The predicted amino acid for these sequences are provided in SEQ ID NO: 14 and SEQ ID NO: 16, respectively.
In addition to B. neritina, B. pacifica and its symbionts were found to include PKS activity and polyketides such as bryopyrans or bryostatins. Larval symbiont identity

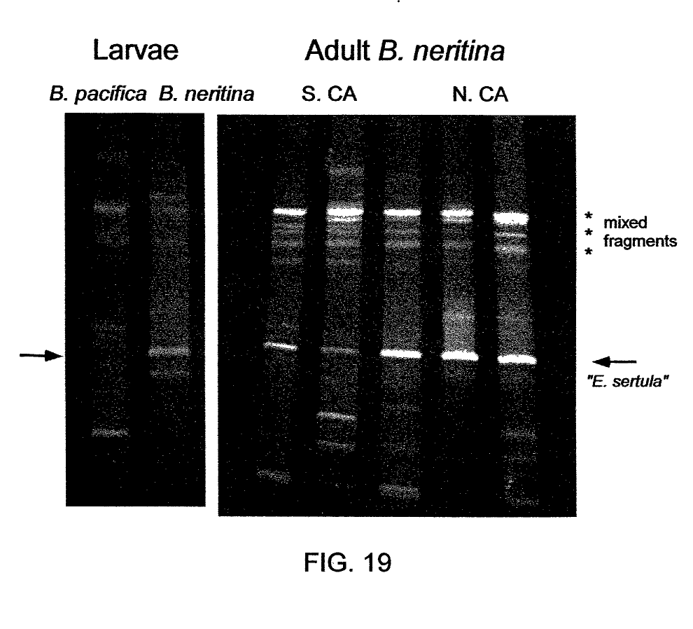

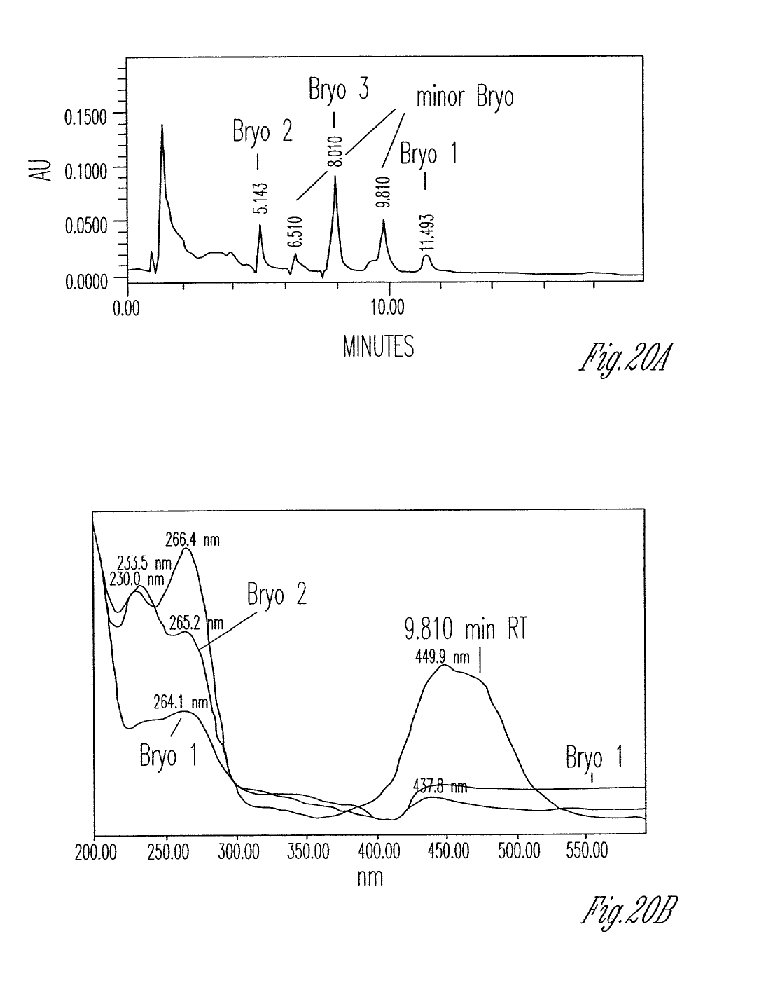

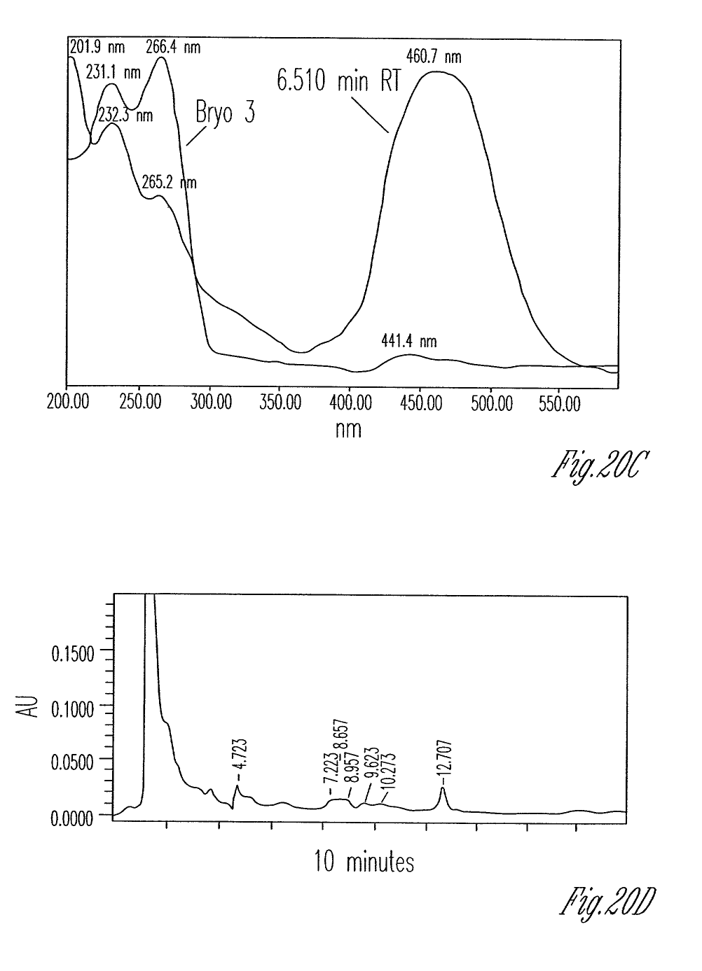

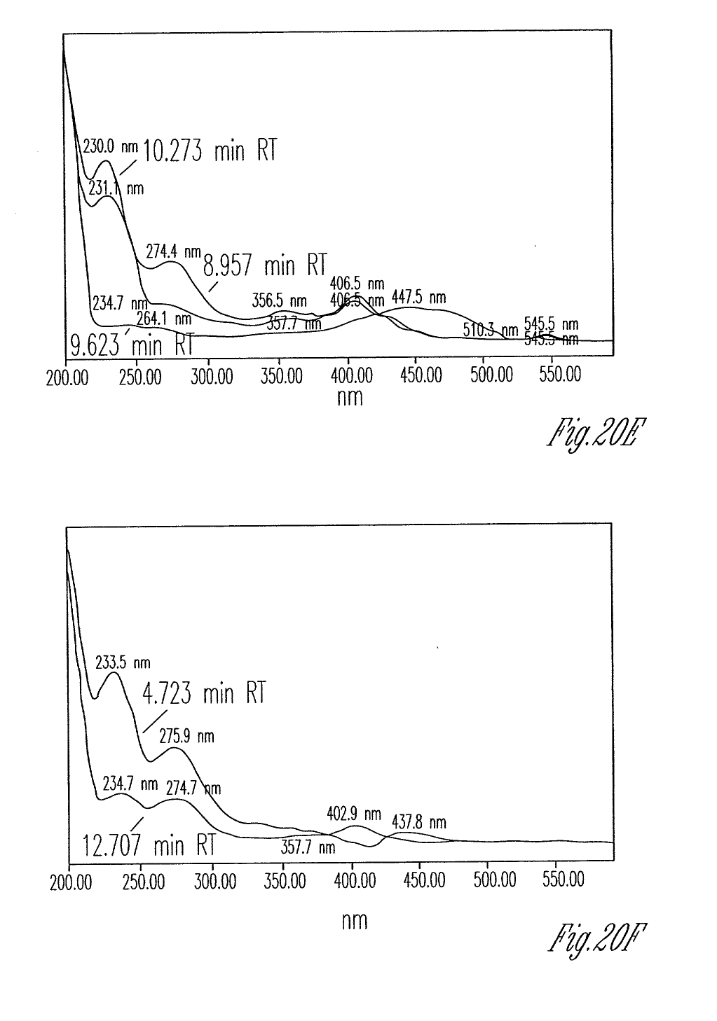

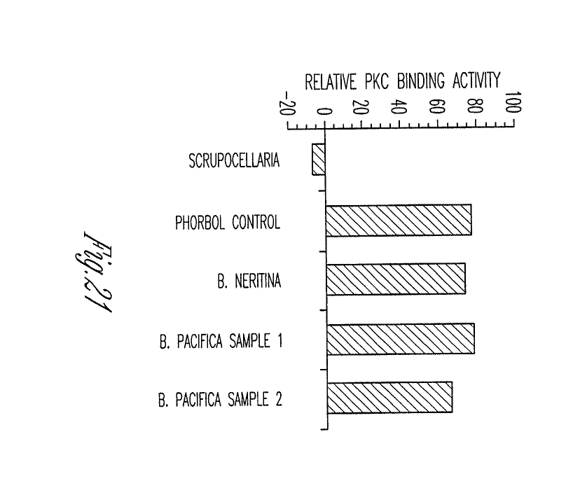

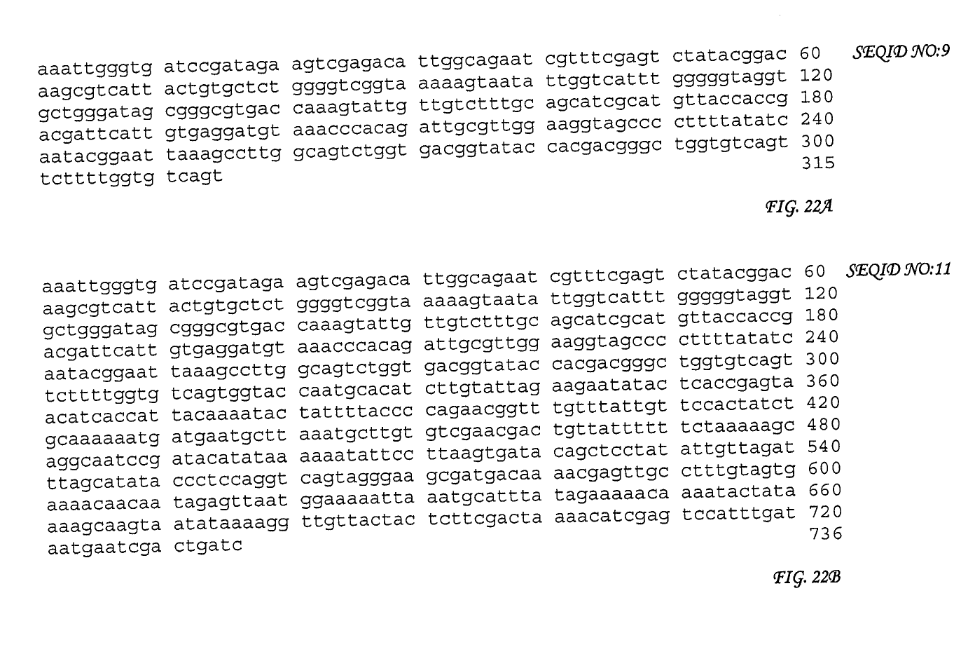

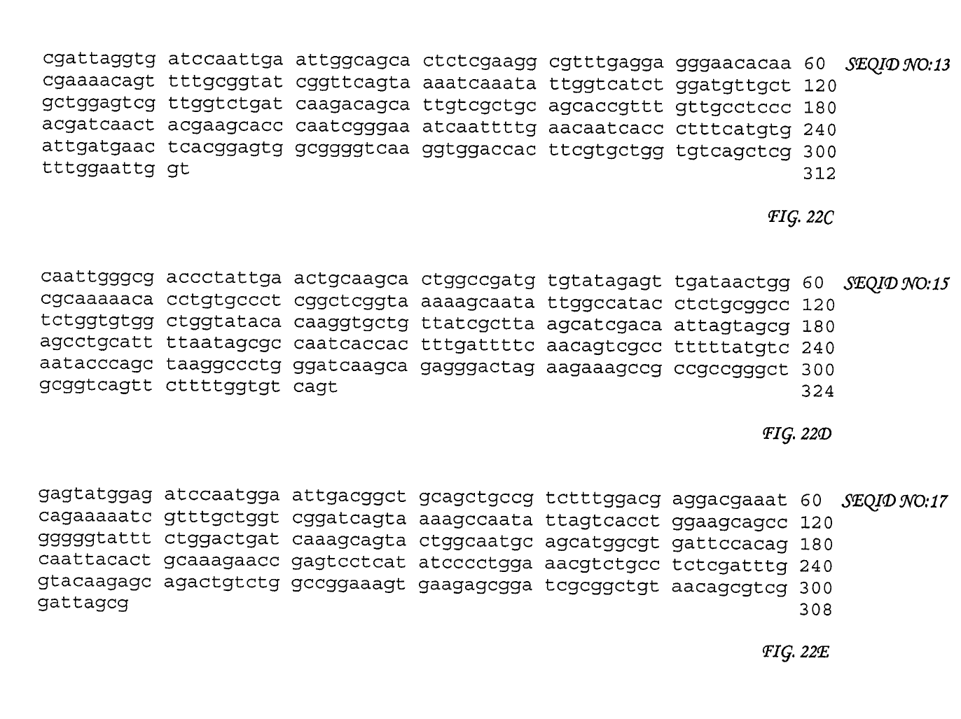

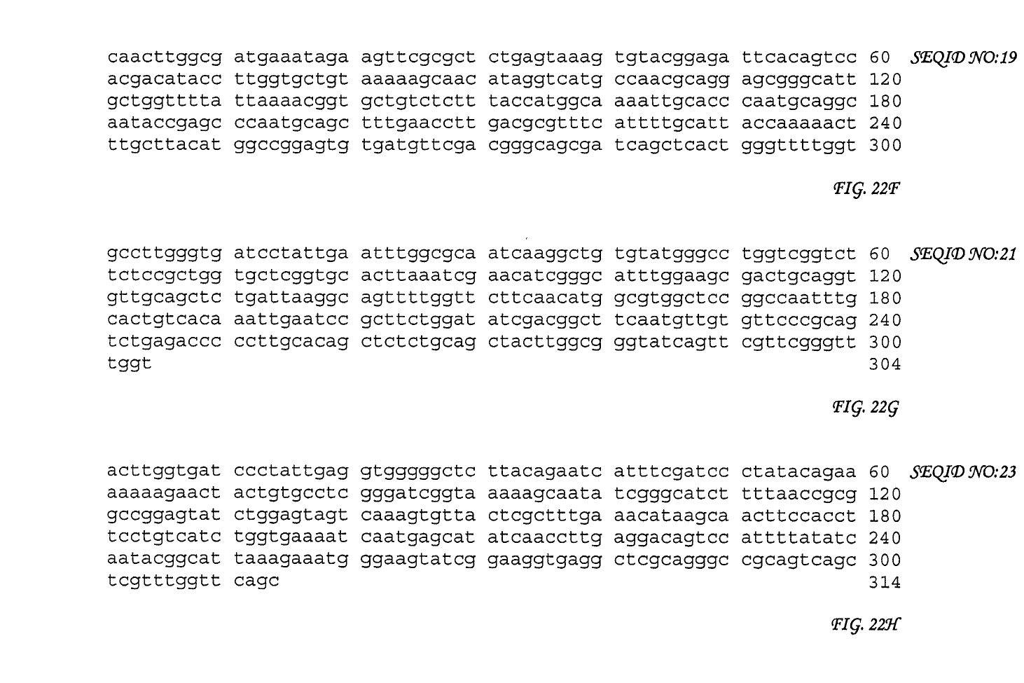

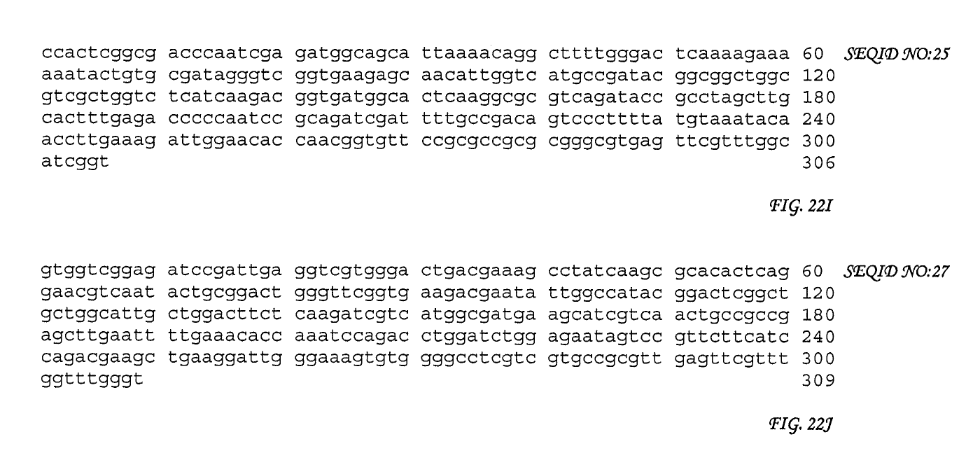

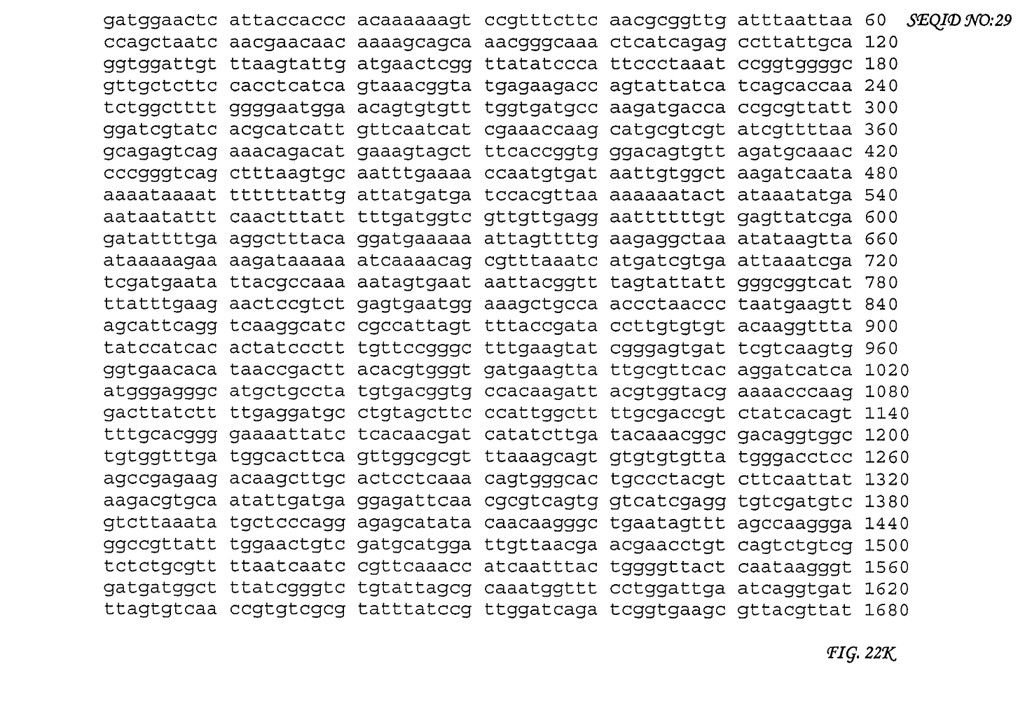

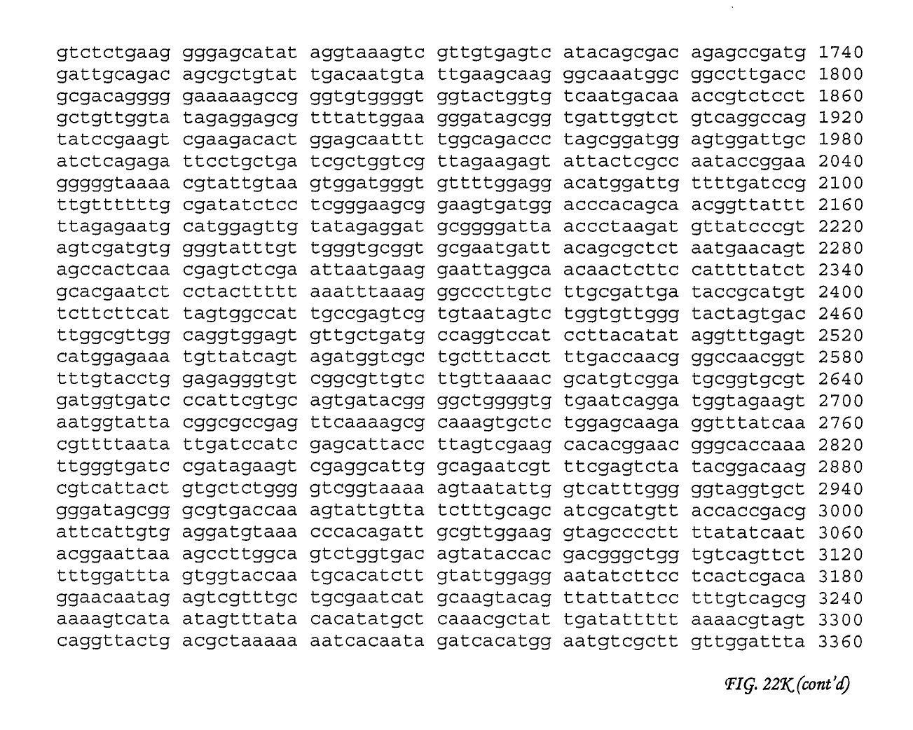

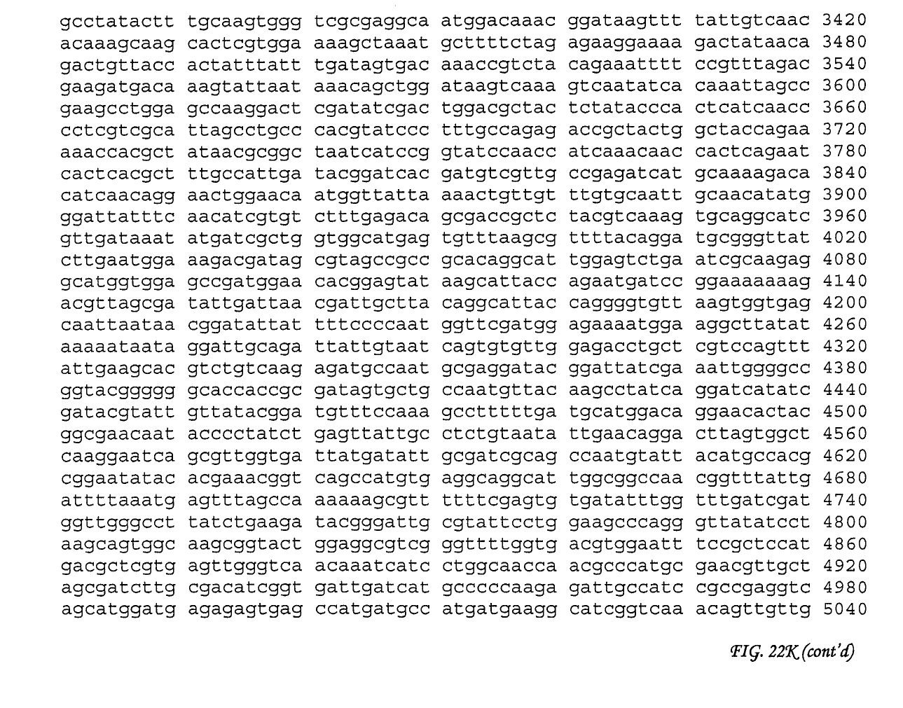

When B. pacifica adult DNA was PCR amplified with the E. sertula specific 16S rRNA primers, no strong band was obtained. This result shows that the priming sites in the variable regions of the 16S rRNA genes of the larval symbiont of B. pacifica differ from those of E. sertula. However, an extremely faint band was present and was cloned. The sequence of the clone is distinct from, but closely related to, E. sertula, the symbiont of B. neritina. The two E. sertula strains (Davidson and Haygood, 1999) differ by 0.6%, the B. pacifica symbiont differs from E. sertula by about 5%, comparable to a species difference within a bacterial genus. This result was confirmed by DGGE of both B. neritina and B. pacifica larval DNA. No band was corresponding exactly to the E. sertula band of B. neritina was seen in the B. pacifica sample, but several other candidate bands were observed (FIG. 19).

Chemistry

B. pacifica extracts were made generally following reported methods (see, for example, Schaufelberger et al., J. Nat. Products, 54:1265-1270 (1991)). These extracts were separated using HPLC, which provided profiles different from that of B. neritina. Five peaks occurred in the region where bryostatins appear; one of the peaks (9.623 min RT) had a retention time and UV absorbance maxima similar to one of the minor bryostatins; the others did not (FIG. 20). These data indicate that molecules with chemical properties similar to the bryostatins may be present and most of them differ significantly from known bryostatins.

Activity

PdBu assay of an extract of B. pacifica colonies showed significant protein kinase C binding activity (DeVries et al., 1988) (FIG. 21).

PKS-I genes

PCR amplification of adult B. pacifica DNA with the degenerate KS primers (PKSR and BLCASPKS) yielded a strong band of approximately 300 bp, similar in size to that obtained from B. neritina DNA. Because the product is a mixture of sequences from different KS domains from different modules of the PKS-I, the band was cloned. Two clones have been sequenced and their deduced amino acid sequence identical to KS clones KSb and KSc from B. neritina (FIG. 24).

All publications, including patent documents and scientific articles, referred to in this application, including any bibliography, are incorporated by reference in their entirety for all purposes to the same extent as if each individual publication were individually incorporated by reference.

All headings are for the convenience of the reader and should not be used to limit the meaning of the text that follows the heading, unless so specified.

### REFERENCES

U.S. Pat. No. 3,960,897 to Story et al., issued Jun. 1, 1976.

U.S. Pat. No. 4,560,774 to Pettit et al., issued Dec. 24, 1985.

U.S. Pat. No. 4,611,066 to Pettit et al., issued Sep. 9, 1986.

U.S. Pat. No. 4,833,257 to Pettit et al., issued May 23, 1989.

U.S. Pat. No. 4,940,726 to Pettit et al., issued Jul. 10, 1990.

U.S. Pat. No. 5,072,004 to Pettit, issued Dec. 10, 1991.

U.S. Pat. No. 5,358,711 to May et al., issued Oct. 25, 1994.

U.S. Pat. No. 5,521,077 to Khosla et al., issued May 28, 1996.

U.S. Pat. No. 5,652,125 to Scotti et al., issued Jul. 29, 1997.

U.S. Pat. No. 5,672,491 to Khosla et al., issued Sep. 30, 1997.

U.S. Pat. No. 5,712,146 to Khosla et al., issued Jan. 27, 1998.

U.S. Pat. No. 5,716,849 to Ligon et al., issued Feb. 10, 1998.

U.S. Pat. No. 5,716,968 to Driedger et al. issued Feb. 10, 1998.

U.S. Pat. No. 5,734,038 to Au-Young et al. issued Mar. 31, 1998.

U.S. Pat. No. 5,744,350 to Vinci et al., issued Apr. 28, 1998.

U.S. Pat. No. 5,750,709 to Castor, issued May 12, 1998.

U.S. Pat. No. 5,776,486 to Castor et al. issued Jul. 7, 1998.

U.S. Pat. No. 5,783,431 to Peterson et al., issued Jul. 21, 1998.

U.S. Pat. No. 5,804,433 to Gray et al., issued Sep. 8, 1998.

U.S. Pat. No. 5,811,285 to Gray et al., issued Sep. 22, 1998.

U.S. Pat. No. 5,824,485 to Thompson et al., issued Oct. 20, 1998.

U.S. Pat. No. 5,824,513 to Katz et al., issued Oct. 20, 1998.

U.S. Pat. No. 5,830,750 to Khosla et al., issued Nov. 3, 1998.

U.S. Pat. No. 5,840,324 to Hennessy et al., issued Nov. 24, 1998.

U.S. Pat. No. 5,843,718 to Khosla et al., issued Dec. 1, 1998.

U.S. Pat. No. 5,849,541 to Vinci et al., issued Dec. 15, 1998.

U.S. Pat. No. 5,876,991 to DeHoff et al., issued Mar. 2, 1999.

WO 93/13663 to Katz et al., published Jul. 22, 1993.

WO 95/12661 to Vinci et al., published May 11, 1995.

WO 97/02358 to Khosla et al., published Jan. 23, 1997.

WO 97/11709 to Piper, published Apr. 3, 1997.

WO 97/22711 to Sherman et al., published Jun. 26, 1997.

WO 97/34598 to Blumberg et al., published Sep. 25, 1997.

WO 97/47294 to Nobuhiro et al., published Dec. 18, 1997.

WO 98/01546 to Leadlay et al., published Jan. 15, 1998.

WO 98/11230 to Oki et al., published Mar. 19, 1998.

WO 98/17811 to Peterson et al., published Apr. 30, 1998.

WO 98/20042 to Wei et al., published May 14, 1998.

WO 98/27203 to Barr et al., published Jun. 25, 1998.

WO 98/27817 to Sirnyan et al., published Jul. 2, 1998.

WO 98/41869 to Nasby et al., published Sep. 24, 1998.

WO 98/48813 to Prichard et al., published Nov. 5, 1998.

WO 98/49294 to Prichard et al., published Nov. 5, 1998.

WO 98/49315 to Khosla et al., published Nov. 5, 1998.

WO 98/53097 to Waters et al., published Nov. 26, 1998.

WO 98/58063 to Hillman et al., published Dec. 23, 1998.

WO 99/02669 to Betlach, published Jan. 21, 1999.

EP 0791655A2 to Dehoff, published Aug. 27, 1997.

EP 0791656A2 to Burgett, published Aug. 27, 1997.

Altschul, S. F., T. L. Madden, A. A. Schaffer, J. Zhang, Z. Zhang, W. Miller and D. J. Lipman. (1997). Gapped BLAST and PSI-BLAST: A new generation of protein database search programs. Nucleic Acids Res., 25: 3389-3402.

Baldwin, N. G., C. D. Rice, T. M. Tuttle, H. D. Bear, J. I. Hirsch and R. E. Merchant. (1997). Ex vivo expansion of tumor-draining lymph node cells using compounds which activate intracellular signal transduction. I. Characterization and in vivo anti-tumor activity of glioma-sensitized lymphocytes. J. Neurooncol., 32: 19-28.

Basu, A. (1998). The involvement of novel protein kinase C isozymes in influencing sensitivity of breast cancer MCF-7 cells to tumor necrosis factor-alpha. Molec. Pharm., 53: 105-111.

Bewley, C. A., N. A. Holland and D. J. Faulkner. (1996). Two classes of metabolites from Theonella swinhoei are localized in distinct populations of bacterial symbionts. Experientia, 52: 716-722.

Caponigro, F., R. C. French and S. B. Kaye. (1997). Protein kinase C: a worthwhile target for anticancer drugs? Anticancer Drugs, 8: 26-33.

Caspi, R., B. M. Tebo and M. G. Haygood. (1998). c-Type cytochromes and manganese oxidation in Pseudomonas putida MnB 1. Applied and Envir. Micro., 64: 3549-3555.

Correale, P., M. Caraglia, A. Fabbrocini, R. Guarrasi, S. Pepe, V. Patella, G. Marone, A. Pinto, A. R. Bianco and P. Tagliaferri. (1995). Bryostatin 1 enhances lymphokine activated killer sensitivity and modulates the beta 1 integrin profile of cultured human tumor cells. Anticancer Drugs, 6: 285-90.

DeVries, D. J., C. L. Herald, G. R. Pettit and P. M. Blumberg. (1988). Demonstration of sub-nanomolar affinity of bryostatin 1 for the phorbol ester receptor in rat brain. Biochem. Pharmacol., 37: 4068-4073.

Dickson, R. B., M. D. Johnson, M. Maemura and J. Low. (1996). Anti-invasion drugs. Breast Cancer Res. Treat., 38: 121-132.

Donadio, S., M. J. Staver, J. B. McAlpine, S. J. Swanson and L. Katz. (1991). Modular organization of genes required for complex polyketide biosynthesis. Science, 759: 675-679.

Fine, R., J. Patel and B. Chabner. (1988). Phorbol esters induce MDR in human breast cancer cells. Proc. Natl. Acad. Sci. USA, 85: 582-586.

Fleming, M. D., H. D. Bear, K. Lipshy, P. J. Kostuchenko, D. Portocarero, A. W. McFadden and S. K. Barrett. (1995). Adoptive transfer of bryostatin-activated tumor-sensitized lymphocytes prevents or destroys tumor metastases without expansion in vitro. J. Immunother. Emphasis Tumor Immunol., 18: 147-55.

Fornwald, J. A., M. J. Donovan, R. Gerber, J. Keller, J. P. Taylor, E. J. Arcuri and M. E. Brawner. (1993). Soluble forms of the human T-cell receptor CD4 are efficiently expressed by Strepomyces lividans. Bio-Technology, 11: 1031-1036.

Grant, S., R. Traylor, G. R. Pettit and P. S. Lin. (1994). The macrocyclic lactone protein kinase C activator, bryostatin 1, either alone, or in conjunction with recombinant murine granulocyte-macrophage colony-stimulating factor, protects Balb/c and C3H/HeN mice from the lethal in vivo effects of ionizing radiation. Blood, 83: 663-667.

Haygood, M. G. and S. K. Davidson. (1997). Small subunit ribosomal RNA genes and in situ hybridization of the bacterial symbionts in the larvae of the bryozoan Bugula neritina and proposal of “Candidatus Endobugula sertula”. Appl. Env. Microbiol., 63: 4612-4616.

Haygood and Davidson (1998). Bacterial symbionts of the byostatin-producing bryozoan Bugula neritina, New. Develop. In Marine Biotechnology, Ed. Le Gal and Halvorson, Plenum Press, New York, pp.281-284.

Haygood (1999), Save the Bugula neritina! It may help beat cancer. San Diego Union-Tribune, Apr. 14, 1999, Current and Arts Section.

Jayson, G. C., D. Crowther, J. Prendiville, A. T. McGown, C. Scheid, P. Stern, R. Young, P. Brenchley, J. Chang, S. Owens and et al. (1995). A phase I trial of bryostatin 1 in patients with advanced malignancy using a 24 hour intravenous infusion. Br J Cancer, 72: 461-8.

Johnson, M. D., J. A. Torri, M. E. Lippman and R. B. Dickson. (1999). Regulation of motility and protease expression in PKC-mediated induction of MCF-7 breast cancer cell invasiveness. Experimental Cell Res., 247: 105-113.

Kageyama, M., T. Tamura, M. H. Nantz, J. C. Roberts, P. Somfai, D. C. Whritenour and S. Masamune. (1990). Synthesis of bryostatin-7. Journal Of the American Chemical Society, 112: 7407-7408.

Kealey, J. T., L. Liu, D. V. Santi, M. C. Betlach and P. Barr. (1998). Production of a polyketide natural product in nonpolyketide producing prokaryotic and eukaryotic hosts. Proc. Natl. Acad. Sci. USA, 95: 505-509.

Korczak, B., C. Whale and R. S. Kerbel. (1989). Possible involvement of Ca++ mobilization and protein kinase C activation in the induction of spontaneous metastasis by mouse mammary adenocarcinoma cells. Cancer Res., 49: 2597-2602.

Kraft, A. S. (1993). Bryostatin 1: will the oceans provide a cancer cure? [editorial; comment]. J. Natl. Cancer Inst., 85: 1790-2.

Kraft, A. S., S. Woodley, G. R. Pettit, F. Gao, J. C. Coll and F. Wagner. (1996). Comparison of the antitumor activity of bryostatins 1, 5, and 8. Cancer Chemother. Pharmacol., 37: 271-8.

Lind, D. S., T. M. Tuttle, K. P. Bethke, J. L. Frank, C. W. McCrady and H. D. Bear. (1993). Expansion and tumour specific cytokine secretion of bryostatin-activated T-cells from cryopreserved axillary lymph nodes of breast cancer patients. Surg. Oncol., 2: 273-82.

Liotta, L. A. and E. Kohn. (1990). Cancer Invasion and Metastases. JAMA, 263: 1123-1126.

Lipshy, K. A., P. J. Kostuchenko, G. G. Hamad, C. E. Bland, S. K. Barrett and H. D. Bear. (1997). Sensitizing T-lymphocytes for adoptive immunotherapy by vaccination with wild-type or cytokine gene-transduced melanoma. Ann. Surg. Oncol., 4: 334-41.

Maloy, S. R., V. J. Stewart and R. K. Taylor. (1996). Genetic Analysis of Pathogenic Bacteria. Plainview, N.Y., Cold Spring Harbor Laboratory Press.

McDaniel, R., A. Thamchaipenet, C. Gustafsson, H. Fu, M. Betlach, M. Betlach and G. Ashley. (1999). Multiple genetic modifications of the erythromycin polyketide synthase to produce a library of novel “unnatural” natural products. Proc. Natl. Acad. Sci. USA, 96: 1846-1851.

Muyzer, G., E. C. De Waal and A. G. Uitterlinden. (1993). Profiling of complex microbial populations by denaturing gradient gel electrophoresis analysis of polymerase chain reaction-amplified genes coding for 16S rRNA. Appl. Environ. Microbiol., 59: 695-700.

Nowak-Thompson, B., S. J. Gould and J. E. Loper. (1997). Identification and sequence analysis of the genes encoding a polyketide synthase required for pyoluteorin biosynthesis in Pseudomonas fluorescens Pf-5. Gene, 204: 17-24.

Ochman, H., M. M. Medhora, D. Garza and D. L. Hartl. (1990). Amplification of flanking sequences by inverse PCR. PCR protocols—a guide to methods and applications. San Diego, Calif., Academic Press.

Pettit, G. R. (1991). The bryostatins. Prog. Chem. Org. Nat. Prod., 57: 153-195.

Pettit, G. R., F. Gao, P. M. Blumberg, C. L. Herald, J. C. Coll, Y. Kamano, N. E. Lewin, J. M. Schmidt and J. C. Chapuis. (1996). Antineoplastic agents. 340. Isolation and structural elucidation of bryostatins 16-18. Journal of Natural Products, 59: 286-9.

Pettit, G. R., F. Gao, D. Sengupta, J. C. Coll, C. L. Herald, D. L. Doubek, J. M. Schmidt, J. R. Van Camp, J. J. Rudloe and R. A. Nieman. (1991). Isolation and structure of bryostatins 14 and 15. Tetrahedron Lett., 22: 3601-3610.

Pettit, G. R., C. L. Herald, D. L. Doubek and D. L. Herald. (1982). Isolation and structure of bryostatin 1. J. Am. Chem. Soc., 104: 6846-6847.

Philip, P. A., Y. Li, M. Alonso, H. Manji, F. Sarkar, J. Ensley and S. Ali-Sadati. (1999). Sensitization of human breast cancer cells to gemcitabine by bryostatin 1. Workshop In: P. o. t. A. A. o. C. R. A. Meeting (ed.), 90th Annual Meeting of the American Association for Cancer Research Philadelphia, Pa.

Pluda, J. M., B. D. Cheson and P. H. Phillips. (1996). Clinical trials referral resource. Clinical trials using bryostatin-1. Oncology (Huntingt), 10: 740-2.

Rangaswamy, V., S. Jiralerspong, R. Parry and C. L. Bender. (1998). Biosynthesis of the Pseudomonas polyketide coronafacid acid requires monofunctional and multifunctional polyketide synthase proteins. Proc. Natl. Acad. Sci. USA, 95: 15469-15474.

Rondon, M. R., S. J. Raffel, R. M. Goodman and J. Handelsman. (1999). Toward functional genomics in bacteria: Analysis of gene expression in Escherichia coli from a bacterial artificial chromosome library of Bacillus cereus. Proc. Natl. Acad. Sci. USA, 96: 6451-6455.

Sambrook, J., E. F. Fritsch and T. Maniatis. (1989). Molecular Cloning: A Laboratory Manual. Plainview, N.Y., Cold Spring Harbor Lab. Press.

Schaufelberger, D. E., M. P. Koleck, J. A. Beutler, A. M. Vatakis, A. B. Alvarado, P. Andrews, L. V. Marzo, G. M. Muschik, J. Roach, J. T. Ross, W. B. Lebherz, M. P. Reeves, R. M. Eberwein, L. L. Rodgers, R. P. Testerman, K. M. Snader and S. Forenza. (1991). The large-scale isolation of bryostatin 1 from Bugula neritina following current good manufacturing practices. J. Nat. Products, 54: 1265-1270.

Scheid, C., J. Prendiville, G. Jayson, D. Crowther, B. Fox, G. R. Pettit and P. L. Stern. (1994). Immunomodulation in patients receiving intravenous Bryostatin 1 in a phase I clinical study: comparison with effects of Bryostatin 1 on lymphocyte function in vitro. Cancer Immunol. Immunother., 39: 223-30.

Schupp, T., C. Toupet, B. Cluzel, S. Neff, S. Hill, J. J. Beck and J. M. Ligon. (1995). A Sorangium cellulosum (Myxobacterium) gene cluster for the biosynthesis of the macrolide antibiotic Soraphen A: cloning, characterization, and homology to polyketide synthase genes from actinomycetes. J. Bacteriol., 177: 3673-3679.

Seow, K. -T., G. Meurer, M. Gerlitz, E. Wendt-Pienkowski, R. Hutchinson and J. Davies. (1997). A study of iterative type II polyketide synthases, using bacterial genes cloned from soil DNA: a means to access and use genes from uncultured microorganisms. J. Bacteriol., 179: 7360-7368.

Shen, Y., P. Yoon, T. -W. Yu, G. G. Floss, D. Hopwood and B. S. Moore. (1999). Ectopic expression of the minimal whiE polyketide synthase generates a library of aromatic polyketides of diverse sizes and shapes. Proc. Natl. Acad. Sci. USA, 96: 3622-3627.

Shure, M., S. Wessler and N. Fedoroff. (1983). Molecular identification and isolation of the waxy locus in maize. Cell, 35: 225-233.

Steube, K. G. and H. G. Drexler. (1993). Differentiation and growth modulation of myeloid leukemia cells by the protein kinase C activating agent bryostatin-1. Leuk. Lymphoma, 9: 141-8.

Sung, S. J., P. S. Lin, R. Schmidt-Ullrich, C. E. Hall, J. A. Walters, C. McCrady and S. Grant. (1994). Effects of the protein kinase C stimulant bryostatin 1 on the proliferation and colony formation of irradiated human T-lymphocytes. Int. J. Radiat. Biol., 66: 775-83.

Taylor, L. S., G. W. Cox, G. Melillo, M. C. Bosco and I. Espinoza-Delgado. (1997). Bryostatin-1 and IFN-gamma synergize for the expression of the inducible nitric oxide synthase gene and for nitric oxide production in murine macrophages. Cancer Res, 57: 2468-73.

Ways, D. K., C. A. Kukoly, J. de Vente, J. L. Hooker, W. O. Bryant, K. J. Posekany, D. J. Fletcher, P. P. Cook and P. J. Parker. (1995). MCF-7 Breast Cancer Cells Transfected with Protein Kinase C-alpha Exhibit Altered Expression of Other Protein Kinase C Isoforms and Display a More Aggressive Neoplastic Phenotype. J. Clin. Invest., 95:

Wender, P. A., J. Debrabander, P. G. Harran, J. M. Jimenez, M. F. T. Koehler, B. Lippa, C. M. Park, C. Siedenbiedel and G. R. Pettit. (1998). The design, computer modeling, solution structure, and biological evaluation of synthetic analogs of bryostatin 1. Proc. Natl. Acad. Sci. USA, 95: 6624-6629.

Wender et al., Synthesis and biological evaluation of fully synthetic bryostatin analogues, Tetrahedron Letters 39:8625-8628 (1998).

Xue, Y., L. Zhao, H. -w. Liu and D. H. Sherman. (1998). A gene cluster for macrolide antibiotic biosynthesis in Streptomyces venezuelae: Architecture of Metabolic Diversity. Proc. Natl. Acad. Sci. USA, 95: 12111-12116.

What is claimed is:

1. A composition, comprising: at least one isolated nucleic acid molecule that encodes at least one polypeptide that catalyzes at least one step in the synthesis of at least one polyketide or bryopyran ring, wherein said at least one nucleic acid is derived from at least one marine organism.

2. The composition of claim 1, wherein said at least one bryopyran ring comprises at least one bryostatin.

3. The composition of claim 1, wherein said at least one polypeptide comprises at least one activity of at least one polyketide synthase.

4. The composition of claim 1, wherein said at least one marine organism comprises at least one bacteria.

5. The composition of claim 4, wherein said at least one bacteria comprises at least one Candidatus.

6. The composition of claim 5, wherein said at least Candidatus comprises at least one Endobugula.

7. The composition of claim 6, wherein said at least one Endobulgula comprises at least one Endobulgula sertula.

8. The composition of claim 1, wherein said at least one marine organism comprises at least one invertebrate.

9. The composition of claim 8, wherein said at least one invertebrate comprises at least one Bugula.

10. The composition of claim 9, wherein said at least one Bugula is Bugula neritina.

11. The composition of claim 9, wherein said at least one Bugula is Bugula pacifica.

12. The composition of claim 1, wherein said at least one nucleic acid molecule further comprises at least one expression control sequence.

13. The composition of claim 1, wherein said nucleic acid molecule is in a vector.

14. The composition of claim 13, wherein said vector is within a cell.

15. The composition of claim 1, wherein said at least one nucleic acid molecule is within a cell.

16. A composition, comprising a library of nucleic acid molecules of claim 1.

17. A composition, comprising: at least one isolated polypeptide that catalyzes at least one step in the synthesis of at least one polyketide or bryopyran ring, wherein said at least one polypeptide is derived from at least one marine organism.

18. The composition of claim 17, wherein said at least one bryopyran ring comprises at least one bryostatin.

19. The composition of claim 17, wherein said at least one polypeptide comprises at least one activity of at least one polyketide synthase.

20. The composition of claim 17, wherein said at least one marine organism comprises at least one bacteria.

21. The composition of claim 20, wherein said at least one bacteria comprises at least one Candidatus.

22. The composition of claim 21, wherein said at least Candidatus comprises at least one Endobugula.

23. The composition of claim 22, wherein said at least one Endobulgula comprises at least one Endobulgula sertula.

24. The composition of claim 17, wherein said at least one marine organism comprises at least one invertebrate.

25. The composition of claim 24, wherein said at least one invertebrate comprises at least one Bugula.

26. The composition of claim 25, wherein said at least one Bugula is Bugula neritina.

27. The composition of claim 25, wherein said at least one Bugula is Bugula pacifica.

28. The composition of claim 17, wherein said at least one polypeptide is within a cell.

29. A composition, comprising a library of polypeptides of claim 17.

30. A method of making a polyketide or bryopyran ring containing composition, comprising: providing the composition of claim 1, and synthesizing a composition therefrom which comprises at least one polyketide or bryopyran ring.

31. The method of claim 30, wherein said at least one bryopyran ring comprises at least one bryostatin.

32. A composition made by the method of claim 30.

33. The composition of claim 32, wherein said composition does not comprise a known bryostatin.

34. The composition of claim 30, comprising at least one pharmaceutically acceptable carrier.

35. The composition of claim 30, wherein said composition is a pharmaceutical composition.

36. A method of making a polyketide or bryopyran ring containing composition, comprising: providing the composition of claim 17, and synthesizing a composition therefrom which comprises at least one polyketide or bryopyran ring.

37. A composition made by the method of claim 36.

38. The composition of claim 37, wherein said composition does not comprise a known bryostatin.

39. The composition of claim 37, comprising at least one pharmaceutically acceptable carrier.

40. The composition of claim 37, wherein said composition is a pharmaceutical composition.

41. A method for identifying at least one nucleic acid molecule encoding at least one activity of a PKS, comprising: contacting a nucleic acid molecule of claim 1 with a sample, and identifying nucleic acid molecules in said sample that hybridize with said nucleic acid molecule of claim 1.

42. The method of claim 41, wherein said sample is derived at least in part from an environmental sample.

43. The method of claim 42, wherein said environmental sample is derived at least in part from a marine environment.

44. A nucleic acid molecule identified by the method of claim 41.

45. The nucleic acid molecule of claim 44, comprising an expression control sequence.

46. The nucleic acid molecule of claim 44 in a vector.

47. The nucleic acid molecule of claim 44 in a cell.

48. A composition comprising a library of nucleic acid molecules of claim 44.

49. A method for identifying a bioactive compound, comprising: contacting the composition of claim 32 with an in vitro, ex vivo or in vivo detection system and determining the bioactivity of said compound.

50. A bioactive compound identified by the method of claim 49.

51. The bioactive compound of claim 50 in a pharmaceutically acceptable carrier.

52. The bioactive compound of claim 50, wherein said bioactive compound is a pharmaceutical compound.

53. A method for identifying a bioactive compound, comprising: contacting the composition of claim 37 and determining the bioactivity of said compound.

54. A bioactive compound identified by the method of claim 53.

55. The bioactive compound of claim 54 in a pharmaceutically acceptable carrier.

56. The bioactive compound of claim 54, wherein said bioactive compound is a pharmaceutical compound.

57. An isolated bacterial symbiont of Bugula, wherein said bacterial symbiont comprises at least one polypeptide that has at least one PKS activity.

58. An isolated bacterial symbiont of B. neritina or B. pacifica, wherein said bacterial symbiont comprises at least one polypeptide that has at least one PKS activity.

59. A composition comprising at least one polyketide, bryopyran ring or bryostatin present in Bugula pacifica.

60. The composition of claim 59, wherein said composition has at least one activity of at least one bryostatin.

61. The composition of claim 59, wherein said composition is isolated from Bugula pacifica.

62. The composition of claim 1 which hybridizes under moderate hybridization conditions to any one of SEQ ID NOS. 9, 11, 13, 15, 17, 19, 21, 23, 25, 27, 29, 30, 31, 32, 33, 34, 35, 36, 37, or the complement thereof.

63. The composition of claim 1 which hybridizes under stringent hybridization conditions to any one of SEQ ID NOS. 9, 11, 13, 15, 17, 19, 21, 23, 25, 27, 29, 30, 31, 32, 33, 34, 35, 36, 37, or the complement thereof.

64. An isolated nucleic acid molecule comprising any one of SEQ ID NOS. 9, 11, 13, 15, 17, 19, 21, 23, 25, 27, 29, 30, 31, 32, 33, 34, 35, 36, 37, or the complement thereof.

65. An isolated nucleic acid molecule encoding SEQ ID NOS. 10, 12, 14, 16, 18, 20, 22, 24, 26, 28, or 38.
